SEQUENCE LISTING (1 sequence)

SEQ ID NO: 1 - protein, 80 aa, Homo sapiens
SDVYCEVCEFLVKEVTKLIDNNKTEKEILDAFDKMCSKLPKSLSEECQEVVDTYGSSILSILLEEVSPELVCSMLHLCSG

(19) United States
(12) Patent Application Publication
(10) Pub. No.: US 2024/0316146 A1
(43) Pub. Date: Sep. 26, 2024

(54) METHODS OF REDUCING NEUROPATHY AND NEUROPATHIC SYMPTOMS AND TREATING CANCER

(71) Applicant: Bexion Pharmaceuticals Inc., Covington, KY (US)

(72) Inventors: Ray Takigiku, Covington, KY (US); Darren P. Wolfe, Cincinnati, OH (US); Anne Mary Noonan, New Albany, OH (US); Robert Wesolowski, Dublin, OH (US); Charles A. Cruze, III, West Chester, OH (US); Gilles Hugues Tapolsky, Boulder, CO (US); Richard Charles Curry, III, Cincinnati, OH (US); Michael Gazda, Cincinnati, OH (US)

(73) Assignee: Bexion Pharmaceuticals Inc, Covington, KY (US)

(21) Appl. No.: 18/260,677

(22) PCT Filed: Jan. 7, 2022
(86) PCT No.: PCT/US22/11731

§ 371 Date: Jul. 7, 2023

Related U.S. Application Data
(60) Provisional application No. 63/135,579, filed on Jan. 9, 2021.
(60) Provisional application No. 63/135,585, filed on Jan. 9, 2021.
(60) Provisional application No. 63/159,809, filed on Mar. 11, 2021.

(51) Int. Cl. A61K 38/17; A61K 9/127; A61K 31/135; A61K 31/137; A61K 31/165; A61K 31/167; A61K 31/192; A61K 31/195; A61K 31/197; A61K 31/282; A61K 31/335; A61K 31/337; A61K 31/381; A61K 31/427; A61K 31/454; A61K 31/4745; A61K 31/475; A61K 31/69; A61K 31/7048; A61K 33/243; A61K 38/07; A61K 39/395; A61K 45/06; A61K 47/24; A61P 35/00

(52) U.S. Cl. CPC A61K 38/1709; A61K 9/127; A61K 31/135; A61K 31/137; A61K 31/165; A61K 31/167; A61K 31/192; A61K 31/195; A61K 31/197; A61K 31/282; A61K 31/335; A61K 31/337; A61K 31/381; A61K 31/427; A61K 31/454; A61K 31/4745; A61K 31/475; A61K 31/69; A61K 31/7048; A61K 33/243; A61K 38/07; A61K 39/3955; A61K 45/06; A61K 47/24; A61P 35/00

(57) ABSTRACT

Disclosed herein are methods for reducing neuropathy and neuropathic symptoms and/or promoting neurogenesis, neuritogenesis, neuroprotection and neuroregeneration using saposin C-phospholipid compositions. Also disclosed are methods for treating cancer by administering a saposin C-phospholipid nanovesicle formulation and one or more antineoplastic agents or immune checkpoint inhibitors, and kits for the treatment of cancer comprising, in separate containers, (a) a saposin C-phospholipid pharmaceutical composition, and (b) a pharmaceutical composition containing an antineoplastic agent or a pharmaceutical composition containing an immune checkpoint inhibitor.

### CROSS-REFERENCE TO RELATED APPLICATIONS

The present invention is filed under 35 U.S.C. § 371 as the U.S. national phase of International Patent Application No. PCT/US22/11731, filed Jan. 7, 2022, which designated the United States and which claims the benefit of U.S. Provisional Application No. 63/135,579, filed on Jan. 9, 2021, U.S. Provisional Application No. 63/135,585, filed on Jan. 9, 2021, and U.S. Provisional Application No. 63/159,809, filed on Mar. 11, 2021, which is hereby incorporated in its entirety including all tables, figures, and claims.

### SEQUENCE LISTING

This application contains a Sequence Listing that has been submitted electronically as an ST26 xml file named “19906002US1 SL.” The xml file, created on Jul. 7, 2023, is 3 KB in size. The material in the xml file is hereby incorporated by reference in its entirety.

### TECHNICAL FIELD

The present disclosure relates to reduction of neuropathy or neuropathic symptoms; promotion of neurogenesis, neuritogenesis, neuroprotection, and neuroregeneration; and treatment of cancer.

### BACKGROUND

Neuropathy is a common disorder of the peripheral nervous system in adults and children, affecting about 2-7% of the world's population (Callaghan et al. (2015) JAMA.; 314:2172-2181). It is associated with acute, chronic or phantom pain, numbness, and muscle weakness, amongst many other symptoms. Neuropathy has various etiologies, including underlying conditions such as autoimmune diseases, tumors, traumatic injuries, infections, metabolic problems, inherited causes and exposure to toxins and medications such as chemotherapy (Hakim et al. (2019), J. Clin & Exp Pharmacol.; 9(4):262) or high doses of many common dietary supplements (eg vitamin B6, pyridoxine).

The prevalence of neuropathy is thought to be even higher in vulnerable cohorts, such as cancer patients receiving chemotherapy. For example, the overall incidence of chemotherapy-induced peripheral neuropathy (CIPN) is estimated to be approximately 38% in patients treated with multiple agents (Hershman (2014) J Clin Oncol; 32:1941-1967). CIPN is a significant side effect that limits dosing and potentially the effectiveness of many of the frontline agents used in the treatment of cancer.

Neuropathy is often associated with nerve injury. While the pathophysiology of peripheral nerve injuries and the mechanisms involved in spontaneous regeneration are beginning to be understood, finding a treatment that significantly improves neurogenesis, neuritogenesis, neuroprotection and/or neuroregeneration after injury has been an elusive goal. In addition, despite the widespread prevalence of neuropathy, treatment options for the condition generally do not treat the underlying cause, instead providing only symptomatic relief. Thus, there is a significant need for additional treatment options for neuropathy. Many neurotoxic agents and treatments are critical for the treatment of many types cancer and cancer itself subtly injures the peripheral nervous system (Housley S N, et al. Cancer Res. 2020 Jul. 1; 80(13):2940-2955)

Treatment of cancer often includes radiation therapy, steroids, and chemotherapy, which often are not well-tolerated by patients. The immune system plays a role in various types of cancer, and cancer immunotherapy involves the attack of cancer cells by a patient's immune system. Often as an alternative to treatments that are not well-tolerated, immunotherapy includes treatment with immune checkpoint inhibitors, which target cancer cells that express, for example, particular proteins such as programmed death-ligand 1 (PDL1). Further, while immunotherapy has been used successfully in subjects with certain cancers, there remains a significant need to fine-tune immunotherapy in order to develop safe and effective treatment options with minimal side effects and maximal efficacy.

Gastrointestinal (GI) cancer, including colorectal cancer, gastric cancer, esophageal cancer, and pancreatic cancer, is a prominent worldwide cause of malignant disease and mortality. Gastric cancer is the fifth most frequently diagnosed cancer and is the third leading cause of cancer death worldwide (Leiting, J. L., & Grotz, T. E. (2019). World J. of Gastrointest. Oncol. 11(9), 652-664). Further, colorectal cancer is the second most common cause of cancer deaths in the United States (American Cancer Society, Colorectal Cancer Facts & Figures 2020-2022. Atlanta, Ga).

GI cancer such as metastatic colorectal cancer (mCRC) has a poor prognosis, with 5-year survival rates of approximately 14% (Siegel R L, et al (2020). CA Cancer J Clin. 70(3):145-64.). Surgery, radiation therapy, and chemotherapy are the key components of colorectal cancer therapy. Certain patients with recurrent and metastatic disease can be salvaged with surgery, but chemotherapy remains the mainstay of therapy for advanced colorectal cancer. The main first- and second-line chemotherapy regimens for mCRC are 5-fluorouracil (5-FU)-/capecitabine-based combination therapies, including: FOLFOX (5-FU, oxaliplatin and folinic acid), FOLFIRI (5-FU, folinic acid, and irinotecan), and CAPOX (capecitabine and oxaliplatin) (Sonbol M B, et al (2019). JAMA Oncol; e194489). Oxaliplatin in FOLFOX causes significant CIPN, and interferes with patients' quality of life, due to which oncologists are frequently forced to decrease or discontinue oxaliplatin doses, which may have a negative impact on disease control and progression-free survival. Selvy M, et al. J Clin Med. 2020; 9(8):2400. Targeted biologic treatments, such as the anti-epidermal growth factor receptor (EGFR) treatments cetuximab and panitumumab, are also recommended for first-line treatment of RAS wild-type mCRC (Fornasier G, et al., (2018) Adv Ther. 35(10):1497-509). Several potential postsecond-line agents exist, including EGFR inhibitors (cetuximab/panitumumab), bevacizumab, regorafenib, and trifluridine/tipiracil (Vogel A, et al. Cancer Treat Rev. 2017; 59:54-60). Each of the agents used in treating mCRC has a significant side-effect profile and offers only modest hope for a successful clinical outcome.

Thus, there remains a significant need for safe and effective treatment options for gastrointestinal cancers, including colorectal cancer, that would have minimal side effects, such as CIPN, while maximizing treatment efficacy.

### SUMMARY

This disclosure is based, at least in part, on at least the following surprising findings:
(1) Saposin C-phospholipid-containing nanovesicle compositions can alleviate symptoms of neuropathy, including in human subjects. Thus, this disclosure provides a novel therapy for the reduction of neuropathy or neuropathic symptoms.
(2) The nanovesicle composition potentiates the anti-cancer effect of oxaliplatin and 5-fluorouracil (5-FU), two components of standard chemotherapeutic regimens for gastrointestinal cancer. The nanovesicle composition also reduced symptoms of CIPN in animals. Thus, this disclosure provides a novel combination therapy for the treatment of gastrointestinal cancer, such as colorectal cancer.
(3) The nanovesicle composition potentiates the anti-cancer effect of an immune checkpoint inhibitor. Thus, this disclosure provides yet another novel combination therapy for the treatment of cancers, e.g., those cancers that are susceptible to treatment with immune checkpoint inhibitors.

In a first aspect, the disclosure features a method of reducing a neuropathic symptom in a human subject, the method comprising: identifying a human subject who is suffering from a neuropathic symptom; administering to the subject a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH; and confirming that the subject's neuropathic symptom is reduced.

In a second aspect, the disclosure features a method of reducing the incidence, intensity, and/or duration of a neuropathic symptom, or delaying onset of a neuropathic symptom, in a human subject in need thereof, the method comprising: identifying a human subject who is at risk of experiencing a neuropathic symptom; and treating the subject with a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH, wherein the treatment is effective in reducing the incidence, intensity, and/or duration of the neuropathic symptom, or delaying onset of the neuropathic symptom in the subject.

In a third aspect, the disclosure features reducing the incidence, intensity, and/or duration of a neuropathic symptom, or delaying onset of a neuropathic symptom, in a human subject in need of treatment with a chemotherapeutic agent that is associated with neuropathic symptom side effects, the method comprising: administering a dose of the chemotherapeutic agent to the subject; and concurrently with the dose of the chemotherapeutic agent, or immediately before or after administering the dose of the chemotherapeutic agent, administering to the subject at least one dose of a nanovesicle formulation comprising (i) a saposin C polypeptide comprising SEQ ID NO: 1 with zero to four amino acid insertions, substitutions, deletions, or combination thereof; and (ii) a phospholipid having a net negative charge at neutral pH.

In a fourth aspect, the disclosure features method of treating cancer, the method comprising co-administering to a human subject in need thereof: a chemotherapeutic agent that is associated with neuropathic symptom side effects; and a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH, wherein the nanovesicle formulation is administered in an amount that reduces the incidence, intensity, and/or duration of the neuropathic symptom side effects associated with the chemotherapeutic agent, or delays onset of the neuropathic symptom side effects associated with the chemotherapeutic agent.

In a fifth aspect, the disclosure features a method of reducing a neuropathic symptom in a human subject in need thereof, the method comprising identifying a subject who is experiencing a neuropathic symptom; and treating the subject with an amount of a nanovesicle formulation effective to reduce the subject's neuropathic symptom, wherein the nanovesicle formulation comprises (i) a saposin C polypeptide comprising SEQ ID NO: 1 with zero to four amino acid insertions, substitutions, deletions, or combination thereof, and (ii) a phospholipid having a net negative charge at neutral pH.

In a sixth aspect, the disclosure features promoting neurogenesis, neuritogenesis, neuroprotection and/or neuroregeneration in a subject, the method comprising: identifying a subject as being in need of one or more of neurogenesis, neuritogenesis, neuroprotection and neuroregeneration; and administering to the subject an amount of a nanovesicle formulation sufficient to promote neurogenesis, neuritogenesis, neuroprotection and/or neuroregeneration in the subject, wherein the nanovesicle formulation comprises (i) a saposin C polypeptide comprising SEQ ID NO: 1 with zero to four amino acid insertions, substitutions, deletions, or combination thereof, and (ii) a phospholipid having a net negative charge at neutral pH. In some embodiments, the subject has one or more of the following conditions: a neurodegenerative disorder, brain damage, brain injury, spinal cord injury, peripheral nerve damage, multiple sclerosis, or disseminated sclerosis. In some embodiments, the subject has Parkinson's disease, Alzheimer's disease, or amyotrophic lateral sclerosis. In some embodiments, the subject is about to undergo anti-cancer chemotherapy. In some embodiments, the method further comprises monitoring neurogenesis, neuritogenesis, neuroprotection and/or neuroregeneration in the subject using one or more tests selected from the Weinstein Enhanced Sensory Test (WEST), Semmes-Weinstein Monofilament Test (SWMT), shape-texture identification (STI), magnetic resonance imaging (MRI), computed tomography (CT), intraepidermal nerve fiber density (IENFD), and nerve conduction velocity. In some embodiments, the method further comprises confirming that the subject experienced neurogenesis, neuritogenesis, neuroprotection and/or neuroregeneration following administration of the formulation.

In some embodiments of the above methods, the subject has the subject has or is at risk of having peripheral neuropathy. In some embodiments, the subject has or is at risk of having peripheral neuropathy. In some embodiments, the subject has or is at risk of having chemotherapy-induced peripheral neuropathy (CIPN) that is induced by one or more chemotherapeutic agents. In some embodiments, the chemotherapeutic agent is selected from the group consisting of a platinum-based agent, a taxane, an epothilone, a plant alkaloid, an immunomodulatory agent, and a proteasome inhibitor. In some embodiments, the chemotherapeutic agent is oxaliplatin, cisplatin, carboplatin, paclitaxel, docetaxel, cabazitaxel, ixabepilone, vinblastine, vincristine, vindesine, vinorelbine, vincaminol, vineridine, vinburnine, etoposide, thalidomide, lenalidomide, pomalidomide, bortezomib, carfilzomib, ixazomib, eribulin or suramin. In some embodiments, the subject has or is at risk of having oxaliplatin-induced acute pain syndrome.

In some embodiments of the first, second, or fifth aspects, the subject has or is at risk of having neuropathy associated with one or more of the following conditions: amyotrophic lateral sclerosis (ALS), spinal muscular atrophy (SMA), rheumatoid arthritis, systemic lupus erythematosus (SLE), post-polio syndrome, Guillain-Barre syndrome, chronic inflammatory demyelinating polyneuropathy (CIDP), multifocal motor neuropathy, muscular dystrophy, peripheral nerve injuries, demyelination, acute disseminated leukoencephalitis, progressive multifocal leukoencephalitis (PML), adrenal leukodystrophy, optic neuritis, kidney disease, liver disorder, transverse myelitis, Parkinson's disease, stroke, Alzheimer's disease, Lyme disease, carpal tunnel syndrome, lymphoma, neuroma, multiple myeloma, vitamin B12 deficiency, post-herpetic neuralgia, leprosy, Charcot-Marie-Tooth disease, Fabry disease, critical illness polyneuropathy, Bell's palsy, ulnar nerve palsy, and peroneal nerve palsy. In some embodiments, the subject has or is at risk of having neuropathy associated with diabetes or a viral infection.

In some embodiments of some of the above aspects, the reduction in neuropathy or neuropathic symptom comprises a change in at least one parameter selected from a reduction in the incidence of neuropathy, a reduction in the intensity of neuropathy, a reduction in the duration of neuropathy, a reduction in the duration of neuropathic episodes, a reduction in the frequency of neuropathic episodes, and delaying onset of neuropathy.

In some embodiments of some of the above aspects, the reduction in neuropathy comprises a reduction in a neuropathic symptom. In some embodiments, the reduction in the incidence, intensity, and/or duration of neuropathy, or the delaying onset of neuropathy, comprises a reduction in the incidence, intensity, and/or duration of a neuropathic symptom, or delaying onset of a neuropathic symptom.

In some embodiments of the second aspect, the method is effective in at least one of (a)-(d): (a) a reduction in the intensity and/or duration of an episode of a neuropathic symptom in the subject after administering the nanovesicle formulation, compared to the intensity and/or duration of episodes of the neuropathic symptom in the subject before the formulation was administered; (b) a reduction in the intensity and/or duration of a neuropathic symptom in the subject, compared to the intensity and/or duration of the neuropathic symptom in a control population; (c) a reduction in the incidence of neuropathy in the subject, compared to the incidence of neuropathy in a control population; and (d) a delay in onset of neuropathy in the subject following a triggering event, compared to the period of time prior to onset of neuropathy in a control population following a similar event.

In some embodiments of some of the above aspects, one or more further doses of the nanovesicle formulation and one or more further doses of the chemotherapeutic agent are administered to the subject. In some embodiments, the nanovesicle formulation does not contain the chemotherapeutic agent.

In some embodiments of the fifth aspect, the neuropathic symptom is associated with (a) one or more of the following conditions: diabetes, amyotrophic lateral sclerosis (ALS), spinal muscular atrophy (SMA), rheumatoid arthritis, systemic lupus erythematosus (SLE), post-polio syndrome, Guillain-Barre syndrome, chronic inflammatory demyelinating polyneuropathy (CIDP), multifocal motor neuropathy, muscular dystrophy, peripheral nerve injuries, demyelination, acute disseminated leukoencephalitis, progressive multifocal leukoencephalitis (PML), adrenal leukodystrophy, optic neuritis, kidney disease, liver disorder, transverse myelitis, Parkinson's disease, stroke, Alzheimer's disease, Lyme disease, a viral infection, carpal tunnel syndrome, lymphoma, neuroma, multiple myeloma, vitamin B12 deficiency, post-herpetic neuralgia, leprosy, Charcot-Marie-Tooth disease, Fabry disease, critical illness polyneuropathy, Bell's palsy, ulnar nerve palsy, or peroneal nerve palsy; or (b) treatment with a chemotherapeutic agent selected from the group consisting of oxaliplatin, cisplatin, carboplatin, paclitaxel, docetaxel, cabazitaxel, ixabepilone, vinblastine, vincristine, vindesine, vinorelbine, vincaminol, vineridine, vinburnine, etoposide, thalidomide, lenalidomide, pomalidomide, bortezomib, carfilzomib, ixazomib, eribulin, and suramin. In some embodiments, the neuropathic symptom is pain. In some embodiments, the method further includes monitoring the subject's neuropathic symptom. In some embodiments, the method further includes confirming that the subject's neuropathic symptom is reduced. In some embodiments, the neuropathic symptom is one or more of the following: pain; hyperalgesia; allodynia; inability to feel pain; inability to feel heat, cold, or physical injury; numbness; hypersensitivity to touch; loss of coordination and proprioception; muscle weakness; muscle wasting; muscle twitching; cramps; or muscle paralysis.

In some embodiments, some of the above methods further comprise administering to the subject one or more additional anti-neuropathy treatments selected from the group consisting of transcutaneous electrical nerve stimulation (TENS), therapeutic plasma exchange (TPE), intravenous immune globulin (IVIG) therapy, physical therapy, a pain reliever, an anti-epileptic agent, a topical treatment, and an anti-depressant agent. In some embodiments, some of the above methods further comprise administering to the subject ibuprofen, gabapentin, pregabalin, capsaicin cream, a lidocaine patch, amitriptyline, doxepin, nortriptyline, duloxetine, or venlafaxine.

In some embodiments of some of the above aspects, the neuropathy or neuropathic symptom is confirmed to be reduced as measured using one or more assessment tools selected from the group consisting of the National Cancer Institute-Common Toxicity Criteria (NCI-CTC), the Numeric Rating Scale (NRS), the Visual Analog Scale (VAS), the European Organization for Research and Treatment of Cancer (EORTC) Qualify of Life (QLQ)-CIPN20, QLC-C30, the Functional Assessment of Cancer Therapy/Gynecologic Oncology Group-Neurotoxicity (FACT/GOG-Ntx), the Total Neuropathy Score (TNS) questionnaire, the Chemotherapy-Induced Peripheral Neuropathy Assessment Tool (CIPNAT), and the Post-Oxiplatin Symptom Survey.

In a seventh aspect, the disclosure features a method of treating gastrointestinal cancer in a subject, the method comprising administering to the subject (a) a chemotherapeutic regimen comprising one or more antineoplastic agents, and (b) a nanovesicle formulation comprising (i) a saposin C polypeptide comprising SEQ ID NO: 1 with zero to four amino acid insertions, substitutions, deletions, or combination thereof; and (ii) a phospholipid having a net negative charge at neutral pH.

In some embodiments of the seventh aspect, the chemotherapeutic regimen comprises modified FOLFOX7 (mFOLFOX7). In some embodiments, the antineoplastic agent comprises oxaliplatin. In some embodiments, the nanovesicle formulation is administered simultaneously with the chemotherapeutic regimen, or within 48 hours before or after administration of the chemotherapeutic regimen begins. In some embodiments, the nanovesicle formulation is administered intravenously. In some embodiments, the chemotherapeutic regimen is administered intravenously. In some embodiments, the gastrointestinal cancer is esophageal cancer, gastric cancer, anal cancer, colorectal cancer, bowel cancer, gallbladder cancer, pancreatic cancer, liver cancer, islet cell cancer, rectal cancer, small intestine cancer, gastrointestinal carcinoid tumors, or gastrointestinal stromal tumors. In some embodiments, the method further comprises administering to the subject a biologic treatment for gastrointestinal cancer. In some embodiments, the method further comprises administering to the subject an anti-vascular endothelial growth factor (VEGF) monoclonal antibody. In some embodiments, the method further comprises administering to the subject an anti-epidermal growth factor receptor (EGFR) antibody. In some embodiments, the method further comprises administering to the subject bevacizumab, cetuximab or panitumumab.

In some embodiments of the seventh aspect, the method comprises (a) administering 2.4 mg/kg of the nanovesicle formulation over a period of about 45 minutes to about 120 minutes on day 1 of week 1 of treatment, and (b) after completion of step (a), administering the chemotherapeutic regimen sequentially in the following steps: (i) 85 mg/m² oxaliplatin infused with 200 mg/m² leucovorin calcium over a period of about 2 hours on day 1 of week 1 of treatment; and (ii) 2400 mg/m² 5-fluorouracil (5-FU) over a period of about 46 hours on days 1 and 2 of week 1 of treatment. In some embodiments of the seventh aspect, the method comprises (a) administering 2.4 mg/kg of the nanovesicle formulation over a period of about 45 minutes to about 120 minutes on day 1 of week 1 of treatment; (b) after completion of step (a), administering the chemotherapeutic regimen sequentially in the following steps: (i) 85 mg/m² oxaliplatin infused with 200 mg/m² leucovorin calcium over a period of about 2 hours on day 1 of week 1 of treatment, and (ii) 2400 mg/m² 5-FU over a period of about 46 hours on days 1 and 2 of week 1 of treatment; and (c) administering about 5 mg/kg to about 15 mg/kg of bevacizumab over a period of about 30 to about 90 minutes on day 1 of week 1 of treatment.

In some embodiments of the seventh aspect, the method further comprises administering an additional dose of the nanovesicle formulation 3 times a week in week 2 of treatment. In some embodiments, the method further comprises administering an additional dose of the nanovesicle formulation once every week (every 7 (+/−3) days) in weeks 3 and 4 of treatment. In some embodiments, the method further comprises administering an additional dose of the nanovesicle formulation every 14 (+/−3) days through weeks 5-23 of treatment. In some embodiments, multiple doses of the nanovesicle formulation are administered over a period of at least 8 weeks. In some embodiments, the nanovesicle formulation is administered at least once per week for the first four weeks of treatment. In some embodiments, the nanovesicle formulation is administered at least three times per week for the first two weeks of treatment. In some embodiments, the nanovesicle formulation is administered at least once every 14 (+/−3) days after the first four weeks of treatment. In some embodiments, the nanovesicle formulation is administered at least once every 14 (+/−3) days in weeks 5-24. In some embodiments, the nanovesicle formulation is administered at least once every 28 (+/−3) days after the first 24 weeks of treatment. In some embodiments, the nanovesicle formulation is administered repeatedly to the subject, as follows: week 1: one dose on each of days 1-5; week 2: one dose every other day for a total of 3 doses; weeks 3 and 4: one dose each week (every 7 (+/−3) days); and weeks 5-24: one dose every 14 days (+/−3 days). In some embodiments of the seventh aspect, the method further comprises further comprising administering an additional dose of the chemotherapeutic regimen in week 3 and once every 14 days (+/−3 days) in weeks 5-24. In some embodiments of the seventh aspect, the method further comprising administering an additional dose of the bevacizumab in week 3 and once every 14 days (+/−3 days) in weeks 5-24.

In some embodiments of the seventh aspect, the nanovesicle formulation is not combined with any antineoplastic agent of the chemotherapeutic regimen when the nanovesicle formulation is administered to the subject.

In some embodiments of the seventh aspect, the treatment increases at least one of the following parameters in the subject relative to a control population treated with the chemotherapeutic regimen and not with the nanovesicle formulation: (a) objective response rate (ORR); (b) overall survival (OS); (c) progression free survival (PFS); and (d) duration of response (DoR).

In an eighth aspect, the disclosure features method of treating cancer in a subject in need thereof, the method comprising administering to the subject: (a) a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH; and (b) an immune checkpoint inhibitor. In some embodiments, the immune checkpoint inhibitor is an anti-CTLA-4 antibody. In some embodiments, the anti-CTLA antibody is selected from the group consisting of ipilimumab, tremelimumab, and a combination thereof. In some embodiments, the immune checkpoint inhibitor is an anti-PD-1 antibody. In some embodiments, the anti-PD-1 antibody is selected from the group consisting of nivolumab, pembrolizumab, pidilizumab, MEDIO680, and combinations thereof. In some embodiments, the immune checkpoint inhibitor is an anti-PD-L1 antibody. In some embodiments, the anti-PD-L1 antibody is selected from the group consisting of atezolizumab, BMS-936559, MEDI4736, MSB0010718C, and combinations thereof.

In some embodiments, the cancer is selected from B cell lymphoma, basal cell carcinoma, bladder cancer, blastoma, brain metastasis, breast cancer, Burkitt's lymphoma, cervical cancer, colon cancer, colorectal cancer, endometrial carcinoma, esophageal cancer, Ewing's sarcoma, fibrosarcoma, follicular lymphoma, gastric cancer, gastroesophageal junction carcinoma, gastrointestinal cancer, glioblastoma, glioma, head and neck cancer, hepatic metastasis, Hodgkin's or non-Hodgkin's lymphoma, kidney cancer, laryngeal cancer, leukemia, liver cancer, lung cancer, lymphoblastic lymphoma, lymphoma, mantle cell lymphoma, metastatic brain tumor, metastatic cancer, myeloma, neuroblastoma, ocular melanoma, oropharyngeal cancer, osteosarcoma, ovarian cancer, pancreatic cancer, prostate cancer, renal cell carcinoma, salivary gland carcinoma, sarcoma, skin cancer, soft tissue sarcoma, solid tumor, squamous cell carcinoma, synovia sarcoma, testicular cancer, thyroid cancer, transitional cell cancer, uveal melanoma, verrucous carcinoma, vulval cancer, and Waldenstrom macroglobulinemia.

In some embodiments, the cancer is gastrointestinal cancer. In some embodiments, one or more cells in the cancer express PD-L1 at an elevated level compared to a reference sample. In some embodiments, one or more cells in the cancer express CTLA-4 at an elevated level compared to a reference sample. In some embodiments, the reference sample is selected from (a) a noncancerous sample from the subject; or (b) a noncancerous sample from a different subject. In some embodiments, the one or more cells in the cancer are the same cell type as cells in the reference sample. In some embodiments, the method further comprises conducting an assay to determine whether the treatment with the nanovesicle formulation and the immune checkpoint inhibitor resulted in an immune response against the subject's tumor that is increased compared to the immune response prior to the treatment. In some embodiments, the assay measures the level of one or more cytokines in plasma of the subject. In some embodiments, the assay measures the level of mRNA encoding one or more cytokines in the subject. In some embodiments, the assay measures the level of tumor-associated M1 or M2 macrophages in the subject. In some embodiments, the assay detects presentation of PD-L1 on the surface of macrophages in the subject. In some embodiments, the assay measures the level of activated cytotoxic T cells in the subject. In some embodiments, the nanovesicle formulation does not comprise the immune checkpoint inhibitor.

In some embodiments of any of the above aspects, the saposin C polypeptide's amino acid sequence comprises SEQ ID NO: 1 with one or two amino acid insertions, substitutions, deletions, or combination thereof. In some embodiments, the saposin C polypeptide's amino acid sequence comprises SEQ ID NO: 1. In some embodiments, the saposin C polypeptide's amino acid sequence consists of SEQ ID NO: 1.

In some embodiments of any of the above aspects, the phospholipid comprises phosphatidylserine. In some embodiments, the phospholipid comprises dioleoyl phosphatidylserine (DOPS) or a salt thereof. In some embodiments, the phospholipid comprises a sodium salt of DOPS. In some embodiments, the phospholipid comprises phosphoglyceride. In some embodiments, the phospholipid comprises one or more of dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, or diphytanoyl phosphatidylserine lipid. In some embodiments, the phosphoglyceride comprises phosphatidate.

In some embodiments of the above aspects, the molar ratio of the phospholipid to the saposin C polypeptide in the nanovesicle formulation is in the range of 8:1 to 20:1. In some embodiments, the molar ratio of the phospholipid to the saposin C polypeptide in the nanovesicle formulation is in the range of 11:1 to 13:1. In some embodiments, the molar ratio of the phospholipid to the saposin C polypeptide in the nanovesicle formulation is 12:1.

In some embodiments of the above methods, the subject has cancer. In some cases, the subject has gastrointestinal cancer, pancreatic cancer, colorectal cancer, bone cancer, brain cancer, sarcoma, neuroblastoma, breast carcinoma, or squamous cell carcinoma.

In some embodiments of any of the above aspects, the nanovesicle formulation is administered intravenously, intra-arterially, intradermally, intramuscularly, intra-cardiacally, intracranially, subcutaneously, intraperitoneally, inhalationally, nasally, orally, or sublingually. In some embodiments, each dose of the nanovesicle formulation administered to the subject contains 0.4 mg/kg to 7 mg/kg of the saposin C polypeptide. In some embodiments, the nanovesicle formulation is administered intravenously. In some embodiments, the nanovesicle formulation is administered at least once a day, once every 2 days, 3 times a week, approximately once every week (every 7 (+/−3) days), or approximately once every 2 weeks (every 14 (+/−3) days). In some embodiments, multiple doses of the nanovesicle formulation are administered over a period of at least 8 weeks.

In a ninth aspect, the above disclosure features a kit for the treatment of cancer, the kit comprising, in separate containers, (a) a first pharmaceutical composition comprising at least one antineoplastic agent; and (b) a second pharmaceutical composition comprising (i) a saposin C polypeptide comprising SEQ ID NO: 1 with zero to four amino acid insertions, substitutions, deletions, or combination thereof, and (ii) a phospholipid having a net negative charge at neutral pH. In some embodiments, the antineoplastic agent in the kit is oxaliplatin.

In a tenth aspect, the above disclosure features a kit for the treatment of cancer, the kit comprising, in separate containers, (a) a first pharmaceutical composition comprising at least one immune checkpoint inhibitor; and (b) a second pharmaceutical composition comprising (i) a saposin C polypeptide comprising SEQ ID NO: 1 with zero to four amino acid insertions, substitutions, deletions, or combination thereof, and (ii) a phospholipid having a net negative charge at neutral pH. In some embodiments, the at least one immune checkpoint inhibitor is an anti-CTLA-4 antibody, an anti-PD-1 antibody, or an anti-PD-L1 antibody.

In some aspects, the disclosure features the use of a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH in any of the methods of treatment described above. In some embodiments, the disclosure features the use of the nanovesicle formulation for reducing a neuropathic symptom in a human subject suffering from a neuropathic symptom. In some embodiments, the disclosure features the use of the nanovesicle formulation for preventing, or for reducing the incidence, intensity, and/or duration of, a neuropathic symptom, or delaying onset of a neuropathic symptom, in a human subject at risk of experiencing a neuropathic symptom. In some embodiments, the disclosure features the use of the nanovesicle formulation for reducing the incidence, intensity, and/or duration of the neuropathic symptom side effects associated with the chemotherapeutic agent, or for delaying onset of the neuropathic symptom side effects associated with the chemotherapeutic agent in a human subject. In some embodiments, the disclosure features the use of the nanovesicle formulation for treating cancer in a human subject administered a chemotherapeutic agent that is associated with neuropathic symptom side effects, wherein the nanovesicle formulation is administered in an amount that prevents or that reduces the incidence, intensity, and/or duration of the neuropathic symptom side effects associated with the chemotherapeutic agent, or delays onset of the neuropathic symptom side effects associated with the chemotherapeutic agent. In some embodiments, the disclosure features the use of the nanovesicle formulation for preventing, or for reducing a neuropathic symptom in a human subject identified as experiencing a neuropathic symptom. In some embodiments, the disclosure features the use of the nanovesicle formulation for promoting neurogenesis, neuritogenesis, neuroprotection and/or neuroregeneration in a subject.

In some embodiments, the disclosure features the use of the nanovesicle formulation described above for treating gastrointestinal cancer in a subject who is co-administered a chemotherapeutic regimen comprising one or more antineoplastic agents. In some embodiments, the use comprises administering the following to the subject: (a) 2.4 mg/kg of the nanovesicle formulation over a period of about 45 minutes to about 120 minutes on day 1 of week 1 of treatment; (b) after completion of step (a), administering the chemotherapeutic regimen sequentially in the following steps: (i) 85 mg/m² oxaliplatin infused with 200 mg/m² leucovorin calcium over a period of about 2 hours on day 1 of week 1 of treatment, and (ii) 2400 mg/m² 5-FU over a period of about 46 hours on days 1 and 2 of week 1 of treatment; and (c) administering 5 mg/kg of bevacizumab over a period of time ranging from about 30 to about 90 minutes on day 1 or 2 of week 1 of treatment. In some embodiments, the use comprises administering the nanovesicle formulation to the subject as follows: week 1: one dose on each of days 1-5; week 2: one dose every other day for a total of 3 doses; weeks 3 and 4: one dose each week (every 7 (+/−3) days); and weeks 5-24: one dose every 14 days (+/−3 days). In some embodiments, the use comprises administering an additional dose of the chemotherapeutic regimen in week 3 and once every 14 days (+/−3 days) in weeks 5-24. In some embodiments, the use comprises administering an additional dose of bevacizumab in week 3 and once every 14 days (+/−3 days) in weeks 5-24.

In some embodiments, the disclosure features the use of the nanovesicle formulation described above for treating cancer in a subject co-administered an immune checkpoint inhibitor.

In some aspects, the disclosure features a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH for use in any of the methods of treatment described above. In some embodiments, the disclosure features the nanovesicle formulation for use in reducing a neuropathic symptom in a human subject suffering from a neuropathic symptom. In some embodiments, the disclosure features the nanovesicle formulation for use in preventing, or for reducing the incidence, intensity, and/or duration of, a neuropathic symptom, or delaying onset of a neuropathic symptom, in a human subject at risk of experiencing a neuropathic symptom. In some embodiments, the disclosure features the nanovesicle formulation for use in reducing the incidence, intensity, and/or duration of the neuropathic symptom side effects associated with the chemotherapeutic agent, or for delaying onset of the neuropathic symptom side effects associated with the chemotherapeutic agent in a human subject. In some embodiments, the disclosure features the nanovesicle formulation for use in treating cancer in a human subject administered a chemotherapeutic agent that is associated with neuropathic symptom side effects, wherein the nanovesicle formulation is administered in an amount that prevents or that reduces the incidence, intensity, and/or duration of the neuropathic symptom side effects associated with the chemotherapeutic agent, or delays onset of the neuropathic symptom side effects associated with the chemotherapeutic agent. In some embodiments, the disclosure features the nanovesicle formulation for use in preventing, or for reducing a neuropathic symptom in a human subject identified as experiencing a neuropathic symptom. In some embodiments, the disclosure features the nanovesicle formulation for use in promoting neurogenesis, neuritogenesis, neuroprotection and/or neuroregeneration in a subject.

In some embodiments, the disclosure features the nanovesicle formulation described above for use in treating gastrointestinal cancer in a subject who is co-administered a chemotherapeutic regimen comprising one or more antineoplastic agents. In some embodiments, the use comprises administering the following to the subject: (a) 2.4 mg/kg of the nanovesicle formulation over a period of about 45 minutes to about 120 minutes on day 1 of week 1 of treatment; (b) after completion of step (a), administering the chemotherapeutic regimen sequentially in the following steps: (i) 85 mg/m² oxaliplatin infused with 200 mg/m² leucovorin calcium over a period of about 2 hours on day 1 of week 1 of treatment, and (ii) 2400 mg/m² 5-FU over a period of about 46 hours on days 1 and 2 of week 1 of treatment; and (c) administering 5 mg/kg of bevacizumab over a period of time ranging from about 30 to about 90 minutes on day 1 or 2 of week 1 of treatment. In some embodiments, the use comprises administering the nanovesicle formulation to the subject as follows: week 1: one dose on each of days 1-5; week 2: one dose every other day for a total of 3 doses; weeks 3 and 4: one dose each week (every 7 (+/−3) days); and weeks 5-24: one dose every 14 days (+/−3 days). In some embodiments, the use comprises administering an additional dose of the chemotherapeutic regimen in week 3 and once every 14 days (+/−3 days) in weeks 5-24. In some embodiments, the use comprises administering an additional dose of bevacizumab in week 3 and once every 14 days (+/−3 days) in weeks 5-24.

In some embodiments, the disclosure features the nanovesicle formulation described above for use in treating cancer in a subject co-administered an immune checkpoint inhibitor.

In any of the above embodiments, the saposin C polypeptide may be a polypeptide comprising or consisting of SEQ ID NO: 1, or may comprise SEQ ID NO: 1 with just one or two amino acid changes. In any of the above embodiments, the phospholipid may comprise dioleoyl phosphatidylserine (DOPS) or a salt thereof. The phospholipid may comprise a sodium salt of DOPS. In any of the above embodiments, the molar ratio of the phospholipid to the saposin C polypeptide in the nanovesicle formulation may be in the range of 8:1 to 20:1. In some embodiments, the molar ratio of the phospholipid to the saposin C polypeptide in the nanovesicle formulation may be in the range of 11:1 to 13:1. In some embodiments, the molar ratio of the phospholipid to the saposin C polypeptide in the nanovesicle formulation may be 12:1.

### BRIEF DESCRIPTION OF THE DRAWINGS

FIGS. 1A and 1B are charts showing the timelines of administration of various treatments to mice in an oxaliplatin chemotherapeutic mouse pain model in two individual studies 1 and 2. In FIG. 1A, male mice in groups A-E of Study 1 were administered 10 mg/kg intraperitoneal oxaliplatin or vehicle at day 0, and administered test article as follows: 10 mL/kg vehicle or 20 mg/kg BXQ-350 on days −3, −2, −1 and days 1 to 9, or 30 mg/kg pregabalin on days 3, 5, 7, and 9. n=12 mice per group. vF: von Frey; CP: Cold Plate. In FIG. 1B, male mice in groups A-E of Study 2 were administered 10 mg/kg intraperitoneal oxaliplatin or vehicle at day 0, and administered test article as follows: 10 mL/kg vehicle, 2 mg/kg BXQ-350 (group C) or 10 mg/kg BXQ-350 (group D) on days −3, −2, −1 and days 1 to 9, or 30 mg/kg pregabalin on days 3, 5, 7, and 9. n=12 mice per group.

FIG. 2 is a bar graph showing the mechanical hypersensitivity via paw withdrawal threshold 50% (g) using von Frey filaments for each of the study groups in Study 1 at baseline (BL) and days 3, 5, 7, and 9 (D3, D5, D7, and D9). *, **, ***, and **** indicate statistically significant difference with p<0.05, p<0.01, p<0.001, and p<0.0001, respectively, when compared to the Oxal/Vehicle group at the same timepoint. Values are shown as mean±SEM. Oxal: oxaliplatin; BXQ: BXQ-350.

FIG. 3 is a line graph showing the functional preservation as a percent change in the paw withdrawal threshold 50% (g) using von Frey filaments for each of groups A-D in Study 1 at days 0, 3, 5, 7, and 9. See Table 2 for study groups A-D. * and ** indicate statistically significant difference with p<0.05 and p<0.01, respectively, when compared to the Oxaliplatin group at the same timepoint. Values are shown as mean±SEM.

FIG. 4 is a bar graph depicting the latency to withdrawal behavior(s) in the cold plate test for each of the study groups in Study 1 at baseline (BL) and days 3, 5, 7, and 9 (D3, D5, D7, and D9). *, **, ***, and **** indicate statistically significant difference with p<0.05, p<0.01, p<0.001, and p<0.0001, respectively, when compared to the Oxal/Vehicle Group in the same timepoint. Values are shown as mean±SEM. Oxal: oxaliplatin; BXQ: BXQ-350.

FIG. 5 is a line graph showing the functional preservation as a percent change in the latency to withdrawal behavior(s) in the cold plate test for each of groups A-D in Study 1 at days 0, 3, 5, 7, and 9. See Table 2 for study groups A-D. ** and *** indicate statistically significant difference with p<0.01 and p<0.001, respectively, when compared to the Oxaliplatin group at the same timepoint. Values are shown as mean±SEM.

FIG. 6 is a bar graph showing the mechanical hypersensitivity via paw withdrawal threshold 50% (g) using von Frey filaments for each of the study groups in Study 2 at baseline (BL) and days 3, 5, 7, and 9 (D3, D5, D7, and D9). The bars in order from left to right for each time point refer to the groups A-E in Study 2. See Table 8 for study groups A-E. *, **, ***, and **** indicate statistically significant difference with p<0.05, p<0.01, p<0.001, and p<0.0001, respectively, when compared to the Veh/Oxal group at the same timepoint. Values are shown as mean±SEM. Veh: vehicle; Oxal: oxaliplatin; BXQ 2: 10 mg/kg vehicle, 2 mg/kg BXQ-350; BXQ 10: 10 mg/kg vehicle, 10 mg/kg BXQ-350; PGB: 30 mg/kg pregabalin.

FIG. 7 is a line graph showing the functional preservation as a percent change in the paw withdrawal threshold 50% (g) using von Frey filaments for groups A-D in Study 2 at days 0, 3, 5, 7, and 9. ** and *** indicate statistically significant difference with p<0.01 and p<0.001, respectively, when compared to the Oxaliplatin group at the same timepoint. Values are shown as mean±SEM. See Table 8 for study groups A-D.

FIG. 8 is a bar graph depicting the latency to withdrawal behavior(s) in the cold plate test for each of the study groups in Study 2 at baseline (BL) and days 3, 5, 7, and 9 (D3, D5, D7, and D9). The bars in order from left to right for each time point refer to the groups A-E in Study 2. See Table 8 for study groups A-E. *, **, ***, and **** indicate statistically significant difference with p<0.05, p<0.01, p<0.001, and p<0.0001, respectively, when compared to the Veh/Oxal Group in the same timepoint. Values are shown as mean±SEM. Veh: vehicle; Oxal: oxaliplatin; BXN 2: 10 mg/kg vehicle, 2 mg/kg BXQ-350; BXQ 10: 10 mg/kg vehicle, 10 mg/kg BXQ-350: BXQ-350; PGB: 30 mg/kg pregabalin.

FIG. 9 is a line graph showing the functional preservation as a percent change in the latency to withdrawal behavior(s) in the cold plate test for groups A-D in Study 2 at days 0, 3, 5, 7, and 9. * and ** indicate statistically significant difference with p<0.05 and p<0.01, respectively, when compared to the Oxaliplatin group at the same timepoint. Values are shown as mean±SEM. See Table 8 for study groups A-D.

FIGS. 10A-10D are images of neurite outgrowth (20× magnification) by NS20Y or PC-12 cells. NS20Y cells were treated with 50 nM BXQ-350 (FIG. 10A) or were untreated (FIG. 10B) in a neurite outgrowth assay. PC-12 cells were treated with 50 nM BXQ-350 (FIG. 10C) or were untreated (FIG. 10D) in a neurite outgrowth assay.

FIGS. 11A-11D are four pairs of images of neurite outgrowth in PC-12 cells. The left image in each pair is a 4× magnification; the right image in each pair is a 20× magnification. BXQ-350 was dosed at 50 nM; oxaliplatin was dosed at 2 μM. The treatments consisted of BXQ-350 alone (FIG. 11A), BXQ-350 and oxaliplatin (FIG. 11B), oxaliplatin alone (FIG. 11C), and untreated cells (FIG. 11D).

FIG. 12 is a bar graph showing the percentage of PC-12 cells with neurite outgrowth under various conditions, i.e., untreated, or after treatment with BXQ-350 (50 nM), oxaliplatin (2 μM), or a combination of BXQ-350 and oxaliplatin. Oxaliplatin alone resulted in less than 1% cell viability, precluding assessment of neuritogenesis.

FIG. 13 is a bar graph showing the percentage of live PC-12 cells compared to the total number of live cells in the untreated control (100%) under various conditions, i.e., untreated or after treatment with BXQ-350 (50 nM), oxaliplatin (2 μM), or a combination of BXQ-350 and oxaliplatin. Oxaliplatin alone resulted in negligible cell viability.

FIGS. 14A and 14B are bar graphs showing the percentage of live PC-12 cells (FIG. 14A) and NS20Y cells (FIG. 14B) with 50 nM BXQ-350 treatment compared to the total number of live cells in the untreated control (normalized to the results for the untreated control, which is shown as 100%). FIGS. 14C and 14D are two pairs of 20× magnified images of neurite outgrowth in PC-12 cells (FIG. 14C) and NS20Y cells (FIG. 14D). The left image in each pair show untreated control cells; the right image in each pair show BXQ-350-treated cells. Values are shown as mean±SEM. * and ** indicate statistically significant difference with p<0.05 and p<0.001, respectively.

FIG. 15A is a bar graph showing the percentage of neurite outgrowth in live PC-12 cells that were treated with 2 μM oxaliplatin, 50 nM BXQ-350, a combination of the two, or untreated for 72 hours. Cells were scored for neurite outgrowth; results are reported in the figure as the percentage of cells found to have outgrowth. FIG. 15B is a bar graph showing viable PC-12 cell counts (normalized to the results for the untreated control, which is shown as 100%) 72 hours after treatment with 2 μM oxaliplatin, 50 nM BXQ-350, or a combination of the two, or untreated. Each sample was run in triplicate. Values are shown as mean±SEM. ** and **** indicate statistically significant difference with p<0.01 and p<0.0001, respectively. FIG. 15C shows four images of cells and neurite outgrowth in PC-12 cells for each treatment group. The images are 20× magnifications. The images from left to right correspond to untreated, oxaliplatin treated, BXQ-350 treated, and BXQ-350 plus oxaliplatin treated cells.

FIG. 16A is a bar graph showing the percentage of neurite outgrowth in live NS20Y cells that were treated with 2 μM oxaliplatin, 50 nM BXQ-350, a combination of the two, or untreated for 72 hours. Cells were scored for neurite outgrowth; results are reported in the figure as the percentage of cells found to have outgrowth. FIG. 16B is a bar graph showing viable NS20Y cell counts (normalized to the results for the untreated control, which is shown as 100%) 72 hours after treatment with 2 μM oxaliplatin, 50 nM BXQ-350, or a combination of the two, or untreated. Each sample was run in triplicate. Values are shown as mean±SEM. **, ***, and **** indicate statistically significant difference with p<0.01, p<0.001, and p<0.0001, respectively. FIG. 16C shows four images of cells and neurite outgrowth in NS20Y cells for each treatment group. The images are 20× magnifications. The images from left to right correspond to untreated, oxaliplatin treated, BXQ-350 treated, and BXQ-350 plus oxaliplatin treated cells.

FIG. 17A is a bar graph showing the percentage of neurite outgrowth in live PC-12 cells that were treated with 10 μM vincristine, 50 nM BXQ-350, a combination of the two, or untreated for 72 hours. Cells were scored for neurite outgrowth; results are reported in the figure as the percentage of cells found to have outgrowth. FIG. 17B is a bar graph showing viable PC-12 cell counts (normalized to the results for the untreated control, which is shown as 100%) 72 hours after treatment with 10 μM vincristine, 50 nM BXQ-350, or a combination of the two, or untreated. Each sample was run in triplicate. Values are shown as mean±SEM. *** and **** indicate statistically significant difference with p<0.001 and p<0.0001, respectively. FIG. 17C shows four images of cells and neurite outgrowth in PC-12 cells for each treatment group. The images are 20× magnifications. The images from left to right correspond to untreated, vincristine treated, BXQ-350 treated, and BXQ-350 plus vincristine treated cells.

FIG. 18A is a bar graph showing the percentage of neurite outgrowth in live NS20Y cells that were treated with 10 μM vincristine, 50 nM BXQ-350, a combination of the two, or untreated for 72 hours. Cells were scored for neurite outgrowth; results are reported in the figure as the percentage of cells found to have outgrowth. FIG. 18B is a bar graph showing viable NS20Y cell counts (normalized to the results for the untreated control, which is shown as 100%) 72 hours after treatment with 10 μM vincristine, 50 nM BXQ-350, or a combination of the two, or untreated. Each sample was run in triplicate. Values are shown as mean±SEM. ** and **** indicate statistically significant difference with p<0.01 and p<0.0001, respectively. FIG. 18C shows four images of cells and neurite outgrowth in PC-12 cells for each treatment group. The images are 20× magnifications. The images from left to right correspond to untreated, vincristine treated, BXQ-350 treated, and BXQ-350 plus vincristine treated cells.

FIG. 19A is a bar graph showing the percentage of neurite outgrowth in live PC-12 cells that were treated with 3 μM paclitaxel, 50 nM BXQ-350, a combination of the two, or untreated for 72 hours. Cells were scored for neurite outgrowth; results are reported in the figure as the percentage of cells found to have outgrowth. FIG. 19B is a bar graph showing viable PC-12 cell counts (normalized to the results for the untreated control, which is shown as 100%) 72 hours after treatment with 3 μM paclitaxel, 50 nM BXQ-350, or a combination of the two, or untreated. Each sample was run in triplicate. Values are shown as mean±SEM. *** and **** indicate statistically significant difference with p<0.001 and p<0.0001, respectively. FIG. 19C shows four images of cells and neurite outgrowth in PC-12 cells for each treatment group. The images are 20× magnifications. The images from left to right correspond to untreated, paclitaxel treated, BXQ-350 treated, and BXQ-350 plus paclitaxel treated cells.

FIG. 20A is a bar graph showing the percentage of neurite outgrowth in live NS20Y cells that were treated with 3 μM paclitaxel, 50 nM BXQ-350, a combination of the two, or untreated for 72 hours. Cells were scored for neurite outgrowth; results are reported in the figure as the percentage of cells found to have outgrowth. FIG. 20B is a bar graph showing viable NS20Y cell counts (normalized to the results for the untreated control, which is shown as 100%) 72 hours after treatment with 3 μM paclitaxel, 50 nM BXQ-350, or a combination of the two, or untreated. Each sample was run in triplicate. Values are shown as mean±SEM. *** and **** indicate statistically significant difference with p<0.001 and p<0.0001, respectively. FIG. 20C shows four images of cells and neurite outgrowth in NS20Y cells for each treatment group. The images are 20× magnifications. The images from left to right correspond to untreated, paclitaxel treated, BXQ-350 treated, and BXQ-350 plus paclitaxel treated cells.

FIGS. 21A and 21B are bar graphs showing the percent cell viability of NS20Y cells (FIG. 21A) and PC-12 cells (FIG. 21B) left untreated or pretreated with 50 nM BXQ-350 for 72 hours. Both groups were then treated with media containing 200 μM H₂O₂ for 24 hours. Values are shown as mean±SEM. * and *** indicate statistically significant difference with p<0.05 and p<0.001, respectively.

FIG. 22 is a bar graph showing the percent cell viability of human HT-29 colorectal cancer cells in response to treatment with oxaliplatin/5-FU plus BXQ-350 (SapC-DOPS) relative to untreated cells. Combination treatment with BXQ-350 plus oxaliplatin/5-FU elicits marked, synergistic cell death of cultured human HT-29 cells. Cells were treated for 72 h with BXQ-350 alone (12 μM); BXQ-350+oxaliplatin (0.3 μM); BXQ-350+5-FU (12 μM); oxaliplatin (0.3 μM)+5-FU (12 μM); or BXQ-350+oxaliplatin+5-FU. n=3 per group. Values are shown as mean±SEM.

FIG. 23A is a graph showing toxicity curves (% viable cells) for six colorectal cancer cell lines dosed with SapC-DOPS. SW480 and SW620 cells were dosed at: 3, 6, 9, 12, 15, 20, 25, 30 μM SapC-DOPS. HT29, HTC116, LoVo and SW48 cells were dosed at: 5, 10, 20, 30, 40, 50 μM. FIG. 23B is a bar graph showing the percentage of cells compared to untreated cells for six colorectal cancer cell lines dosed with FOLFOX (10 μM oxaliplatin with 10 μM 5-FU).

FIG. 24 is a bar graph showing the percentage of cells compared to untreated cells for six colorectal cancer cell lines dosed with 15 μM SapC-DOPS alone, 10 μM FOLFOX alone, or a combination of 10 μM FOLFOX and 15 μM SapC-DOPS (Triple Combo). Values are shown as mean±SEM.

FIGS. 25A and 25B are line graphs showing the TNF-alpha concentration (left Y-axis, dark shade line (2)) and percent cell viability (right Y-axis, light shade line (1)) of human CD14+ macrophages from two healthy subjects (FIG. 25A and FIG. 25B, respectively) in response to in vitro treatment with BXQ-350 (SapC-DOPS). Cells were treated with BXQ-350 at concentrations ranging from 2.5 μM to 9 nM, followed by lipopolysaccharide 1 hour later. After 72 hours, the cells were fixed and analyzed for TNF-alpha concentration and percent cell viability. Values are shown as mean±SEM.

FIG. 26 is a series of line graphs showing fluorescence intensities (Incucyte® analyzer) of three dimensional A549 tumor spheroids in the presence of PMBCs stimulated to promote cytotoxic T cells, as a function of time. Line (12): control (no PMBC activation); line (11): activation with anti-CD3+IL-2; line (13): activation with anti-CD3+IL-2 and treatment with pembrolizumab; all other lines (lines (1)-(10)): tumor killing curves for BXQ-350 at concentrations ranging from 1.27 nM to 25 μM and activation with anti-CD3+IL-2.

FIG. 27 is a line graph showing AUC at various concentrations of BXQ-350, based on data from the series of line graphs in FIG. 26. The fluorescence intensity from FIG. 26 is integrated; as cells die, the fluorescence changes as does the total integrated fluorescence which results in different AUC values. AUC: area under the curve. IC50: half maximal concentration necessary to stimulate cell death using BXQ-350.

FIG. 28 is a line graph showing tumor volume of different treatment groups over time after administering sterile PBS (line (1)), m-SapC-DOPS (line (2)), m-anti-PD-1 (line (3)), or both m-SapC-DOPS and m-anti-PD-1 (line (4)) to female BALB/c mice bearing CT-26 established tumors. Animals were sacrificed when tumor volume exceeded 3000 mm³. IV: intravenous injection; IP: intraperitoneal injection; m-SapC-DOPS: mouse SapC-DOPS; D #: day number; mpk: mg/kg; BIW: twice weekly.

FIG. 29 is a line graph showing body weight of different treatment groups over time after administering sterile PBS (line (1)), m-SapC-DOPS (line (2)), m-anti-PD-1 (line (3)), or both m-SapC-DOPS and m-anti-PD-1 (line (4)) to female BALB/c mice bearing CT-26 established tumors.

FIG. 30 is a line graph showing tumor volume of individual animals of different treatment groups over time after administering m-anti-PD-1 (circles, dark shade line) or both m-SapC-DOPS and m-anti-PD-1 (diamonds, light shade line) to female BALB/c mice bearing CT-26 established tumors.

FIGS. 31A-31H show various bar graphs. FIG. 31A is a bar graph showing an increase of IFN-γ concentration in culture medium of cultured myeloid-derived suppressor cells from a healthy donor as a function of BXQ-350 concentration. “T”: T cells without stimulation; “T+Beads”: T cells stimulated to express biomarker (here: interferon-γ). FIGS. 31B and 31C are bar graphs showing percentages of proliferated CD4+ cells and CD8+ cells, respectively, as a function of BXQ-350 concentration. FIG. 31D is a bar graph showing the concentration of IL-10 in media from cultured myeloid-derived suppressor cells from a healthy donor as a function of BXQ-350 concentration. FIGS. 31E-31H are bar graphs showing expression of HLA-DR, CD86, CD11b, and CD80, respectively, on cultured myeloid-derived suppressor cells from a healthy donor as a function of BXQ-350 concentration.

### DETAILED DESCRIPTION

The present disclosure relates to methods for reducing neuropathy or neuropathic symptoms and methods of promoting neurogenesis, neuritogenesis, neuroprotection and/or neuroregeneration. The disclosure also relates to methods for treating cancer. In some embodiments, the disclosure relates to methods for treating gastrointestinal cancer. The compositions used in these methods include a saposin polypeptide, such as saposin C (SapC), and a phospholipid, such as a phosphatidylserine, for example dioleoylphosphatidylserine (DOPS). These saposin-based formulations, as well as their components and preparation techniques, are described in U.S. Pat. No. 10,682,411, the disclosure of which is incorporated herein by reference in its entirety. When used for treating cancer, e.g., gastrointestinal cancer, the saposin C/phospholipid composition is administered with one or more antineoplastic agents, such as a chemotherapeutic agent or an immune checkpoint inhibitor.

A “deletion,” as the term is used herein, refers to a change in the amino acid or nucleotide sequence that results in the absence of one or more amino acid residues or nucleotides.

The words “insertion” or “addition,” as used herein, refer to changes in an amino acid or nucleotide sequence resulting in the addition of one or more amino acid residues or nucleotides, respectively, to the sequence found in the naturally occurring molecule.

### SaposinC-DOPS

Nanovesicles comprising saposin C (“SapC”) and the phospholipid dioleoyl phosphatidylserine (DOPS) have high affinity for phosphatidylserine-rich membranes in vitro and in vivo, and can induce apoptosis and/or necrosis in target cells (Qi et al. (2009), Clin Cancer Res., 15: 5840-5851). The proposed mechanism by which the SapC-DOPS nanovesicles induce apoptosis is via ceramide elevation through activation of 0-glucosidase and acid sphingomyelinase (with subsequent degradation of glucosylceramide and sphingomyelin, respectively), which leads to activation of caspases. The nanovesicle preparation was found to be efficacious against a wide variety of tumor types in vitro and in orthotopic murine tumor models (Qi X, et al. (2009) Clin Cancer Res., 15(18):5840-51; Wojton et al. (2013), Mol Ther, 21: 1517-1525; Abu-Baker et al. (2012), J Cancer Ther, 3: 321-326; Chu et al. (2013), PLoS One; 8: e75507; U.S. Pat. No. 7,834,147).

### Active Agent

The “active agent” used in the present methods is a combination of a saposin C (“SapC”) polypeptide and a phospholipid, which together form lipid vesicles (also termed “liposomes” or “nanovesicles”) when suspended in aqueous solution. Such a suspension is sometimes referred to as a “nanovesicle formulation.” The amino acid sequence of naturally occurring human SapC is: Ser Asp Val Tyr Cys Glu Val Cys Glu Phe Leu Val Lys Glu Val Thr Lys Leu Ile Asp Asn Asn Lys Thr Glu Lys Glu Ile Leu Asp Ala Phe Asp Lys Met Cys Ser Lys Leu Pro Lys Ser Leu Ser Glu Glu Cys Gln Glu Val Val Asp Thr Tyr Gly Ser Ser Ile Leu Ser Ile Leu Leu Glu Glu Val Ser Pro Glu Leu Val Cys Ser Met Leu His Leu Cys Ser Gly (SEQ ID NO:1). The SapC polypeptide used in the present methods consists of or comprises the amino acid sequence of SEQ ID NO: 1, or comprises SEQ ID NO: 1 with one to four amino acid alterations (insertions, substitutions, deletions, or combination thereof, up to a total of four or fewer): for example, an insertion of one to four amino acid residues into SEQ ID NO: 1; or substitution of one, two, three, or four residues in SEQ ID NO: 1; or a deletion of one, two, three, or four residues from the amino terminus, or from the carboxy terminus, or at an internal site of SEQ ID NO: 1. SEQ ID NO: 1 was disclosed as SEQ ID NO:1 in the issued U.S. Pat. No. 10,682,411, which is incorporated by reference in its entirety.

Phospholipids that can be incorporated into the active agent include phospholipids that have a net negative charge at neutral pH, e.g., phosphoglycerides such as phosphatidate (diacylglycerol 3-phosphate) and phosphatidylserine. The two fatty acids attached to the phosphoglyceride backbone can be the same or different; can have, e.g., zero, one, or two carbon-carbon double bonds in each carbon chain; and can have carbon chains from C6 up to C20, e.g., oleic acid, hexanoic acid, octanoic acid, decanoic acid, lauric acid, myristoic acid, stearic acid, palmitic acid, linoleic acid, and phytanic acid, and combinations of any two of these. Thus, where the phospholipid is phosphatidyl serine, the following are typical examples: dioleoyl phosphatidylserine (DOPS), dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, and diphytanoyl phosphatidylserine lipid. The active agent can include a combination of any two or more (e.g., two or three) different phospholipids, such as two or three different phosphatidylserine lipids. (The terms “phosphatidylserine” and “phosphatidylserine lipid” are used interchangeably.)

According to the present disclosure, the active agent may comprise SapC (SEQ ID NO: 1) and DOPS. Alternatively, an anionic phospholipid or phospholipid with an overall negative charge may be used instead of DOPS. SapC-DOPS is described in U.S. Pat. No. 7,834,147, incorporated herein by reference in its entirety. The SapC and DOPS combined may form a nanovesicle. The nanovesicles may be 10 nm to 800 nm, such as 40 nm to 200 nm. A formulation comprising the active agent may have a pH of 5 to 8, such as 7 to 7.4.

In aqueous compositions at neutral pH, phospholipids typically exist in the form of a salt with a cation, and so references to DOPS and other phospholipids used in the present compositions are meant to include both the salt and non-salt forms of the phospholipids. Suitable cations include any pharmaceutically acceptable cation that forms a salt with the phospholipid, such as any of the following: ammonium ion; L-arginine ion; benzathine ion; deanol ion; diethanolamine (2,2′-iminodiethanol) ion; hydrabamine ion; lysine ion; potassium ion; sodium ion; triethanolamine (2,2′,2″-nitrilotri(ethan-1-ol)) ion; and tromethamine (2-amino-2-(hydroxymethyl)propane-1,3-diol) ion. The sodium, potassium, and ammonium salts are typical.

The molar ratio of the SapC polypeptide to the phospholipid in a composition of the invention can be in the range from 1:2 to 1:50, for example 1:5 to 1:30, or 1:8 to 1:20, or 1:11 to 1:13. Suitable molar ratios include but are not limited to 1:10, 1:11, 1:12, 1:13, 1:14, and 1:15. The mass ratio of the polypeptide to the phosphatidylserine lipid is in the range from about 1:0.11 to 1:4.8, or about 1:0.18 to about 1:4.5, or about 1:0.45 to about 1:2.7, or about 1:0.72 to about 1:1.81, or about 1:1 to about 1:1.2.

The active agent may be supplied in the form of a solid (e.g., a lyophilized powder) with or without pharmaceutically acceptable buffers and other inactive ingredients. The solid is typically reconstituted in sterile water or saline before administration, forming a suspension of liposomes in aqueous solution.

When the active agent is in the form of liposomes suspended in an aqueous solution, that solution can also contain pharmaceutically acceptable buffers and other inactive ingredients. Suitable formulations (and methods for preparing them) include those described in U.S. Pat. No. 10,682,411, which is incorporated by reference in its entirety. One example of such a suspension of the active agent in aqueous solution is designated “BXQ-350”. It contains a saposin C polypeptide consisting of SEQ ID NO: 1 and a phospholipid consisting of a sodium salt of DOPS in a molar ratio (SapC to DOPS) of approximately 1:12 (i.e., in the range of 1:11 to 1:13).

Administration of the active agent is typically via injection (e.g., infusion) of the suspension, and may be by any suitable injectable route, e.g., intravenous, intra-arterial, intradermal, intramuscular, intra-cardiac, intracranial, transdermal, subcutaneous, ocular, or intraperitoneal. In appropriate situations, dry particles of the active agent, or a solution containing the active agent, could be aerosolized or nebulized and delivered via inhalation through the nose or mouth, or the solution could be delivered as liquid, e.g., eye drops, nasal drops or spray. Also contemplated is oral delivery, e.g., as a mouthwash or gargle or sublingual formulation, or swallowed as a liquid, capsule, or tablet. The liquid could be delivered as a lavage or enema. Further details regarding routes of administration can be found, for example, in U.S. Pat. No. 7,834,147.

Administration can occur at least once a day for some number of consecutive days, e.g., for 3, 4, 5, 6, 7, 8, 9, or more consecutive days, or can be, e.g., every other day, or 3 times a week, or once every 7±3 days, or once every 14±3 days, or once every 28±3 days. The timing of administrations can start with one of those schedules and after a suitable period of treatment change to another that is more or less frequent. The entire period of treatment can be completed in, e.g., eight or twelve or sixteen weeks, or up to six months, but may continue as long as the patient appears to be benefiting from the treatment.

The SapC-DOPS formulation for use in the present methods may be supplied in lyophilized form and then reconstituted with water before use. An example of a formulation for use in the present methods is shown in Table 1. The molar ratio of DOPS to SapC in the Table 1 formulation is 12:1. The target pH is 7.2±0.4.

TABLE 1
Reconstituted SapC-DOPS formulation

| Component | Concentration | |
|---|---|---|
| SapC | 2.2 | mg/mL |
| DOPS (Na salt) | 2.4 | mg/mL |
| Tris | 25 | mM |

| | |
|---|---|
| Trehalose Dihydrate | 5% |

The formulations for use in the disclosed methods may comprise compositions disclosed in U.S. Pat. No. 10,682,411, the teachings of which are incorporated by reference in its entirety.

SapC and polypeptides derived therefrom may be produced by any useful method, such as chemically, enzymatically, or recombinantly. Methods for producing polypeptides and fragments thereof are known in the art and include, but are not limited to, chemical peptide synthesis, in vitro translation systems, and expression in (and purification from) a recombinant host organism.

Useful background information and technical details can be found in U.S. Pat. Nos. 7,834,147 and 9,271,932, which are incorporated herein by reference in their entirety.

“Lipid vesicle,” “liposome,” and “nanovesicle” are used interchangeably to refer to a generally spherical cluster or aggregate of amphipathic lipids, typically in the form of one or more concentric layers, for example, bilayers.

As used herein, the term “SapC-DOPS” refers to the combination of SapC and DOPS.

As used herein, when a dosage of SapC-DOPS is reported, the dosage refers to the dose of SapC. For example, a dosage of SapC-DOPS of 2.4 mg/kg refers to 2.4 mg/kg of SapC.

“Patient,” “subject,” or “individual” refers to an animal, including a mammal, preferably a human, a monkey, a chimpanzee, a horse, a cow, a sheep, a goat, a pig, a cat, a dog, a mouse, a rat, or a rabbit.

A “therapeutically effective dose” or “therapeutically effective amount” of the active agent, in the context of treating neuropathy, is an amount useful to treat a patient's neuropathy. In general, a single therapeutically effective dose of the present composition will contain an amount of SapC (or its derivative) in the range of about 0.01 to 30 mg/kg body weight, preferably about 0.05 to 20 mg/kg body weight, more preferably about 0.1 to 15 mg/kg body weight, and even more preferably about 0.5 to 10 mg/kg. For example, the amount of SapC in a single intravenous dose can be about 0.4 mg/kg, 0.7 mg/kg, 1.1 mg/kg, 1.4 mg/kg, 1.8 mg/kg, 2.4 mg/kg, 2.8 mg/kg, 3.0 mg/kg, 3.2 mg/kg, 3.6 mg/kg, 7 mg/kg or more. A given patient may receive a given dose level for one or more initial administrations and a different (lower or higher) level for further administrations.

### Neuropathy

“Peripheral neuropathy” is a constellation of different clinical presentations, natural histories, and pathologies that affect the peripheral nervous system (PNS). The PNS consists of sensory neurons running from stimulus receptors that inform the central nervous system (CNS) of the stimuli, and motor neurons running from the spinal cord to the effectors that take action. The term “neuropathy” also includes peripheral symmetric neuropathy, autonomic neuropathy, proximal neuropathy, focal neuropathy, and mononeuropathy.

Patients with peripheral neuropathy present with motor insufficiency (weakness), sensory abnormalities (numbness, parethesias, hyperalgesia/allodynia, pain), autonomic symptoms, or a combination of all, often depending on the particular disease (Cashman, C. R., & Höke, A. (2015) Neuroscience letters, 596, 33-50). While most neuropathies are chronic, slowly progressive conditions, some neuropathies have a more acute onset and gradual recovery (Kuwabara S, Yuki N. (2013) Lancet Neurol. 2013; 12:1180-1188; Winer J B. (2014) Autoimmune Dis.; 2014:1-6; Yuki N, and Hartung H-P. (2012) N. Engl. J. Med. 2012; 366:2294-2304). Few neuropathies are present in isolation, but, rather, are often secondary to other systemic illnesses, including diabetes and infections such as human immunodeficiency virus and hepatitis C virus. Additionally, peripheral neuropathies may be iatrogenic, arising from the toxicity of drugs given as part of antiretroviral or chemotherapy regimens. In chemotherapy-induced peripheral neuropathy (CIPN), an anticancer drug could impair both sensory and motor functions.

Symptoms of neuropathy (“neuropathic symptoms”) include but are not limited to pain, hyperalgesia, allodynia, inability to feel pain, inability to feel heat, cold, or physical injury, hypersensitivity to touch, loss of coordination and proprioception, muscle weakness, muscle wasting, muscle twitching, muscle paralysis, tingling fingers or hands, tingling toes or feet, numbness in fingers or hands, numbness in toes or feet, shooting or burning pain in fingers or hands, shooting or burning pain in toes or feet, cramps in hands and/or feet, problems standing or walking because of difficulty feeling the ground under the feet, difficulty distinguishing between hot and cold water, problem holding a pen and difficulty with writing, difficulty manipulating small objects with fingers (e.g., fastening small buttons), difficulty opening ajar or bottle because of weakness in hands, difficulty walking because feel dropped downwards, difficulty climbing stairs or getting up out of a chair because of weakness in legs, dizziness when standing from a sitting or lying position, blurred vision, difficulty hearing, difficulty using the pedals in a car, and difficulty getting and maintaining an erection. A “neuropathic symptom side effect” is a neuropathic symptom that occurs as a side effect of (i.e., is associated with) the use of chemotherapeutic agents, antiviral drugs, toxic agents such as pesticides, etc.

Neuropathy is a leading cause of chronic pain, which is a pain that persists for three months or more. An estimated 10% of the population has neuropathic pain. “Neuropathic pain” is defined by the International Association For The Study Of Pain (IASP) as “pain initiated or caused by a primary lesion or dysfunction in the nervous system” (IASP, Classification of chronic pain, 2nd Edition, IASP Press (2002), p. 210). In some embodiments of the presently disclosed methods, neuropathic pain results from chemotherapy, i.e., it is caused by administration of a chemotherapeutic agent in chemotherapy. The chemotherapeutic agent can cause neurotoxicity, especially peripheral neurotoxicity resulting in peripheral neuropathy. Allodynia (pain due to a stimulus that does not usually provoke pain) and hyperalgesia or hypersensitivity (increased pain from a stimulus that usually provokes a lower degree of pain) are prominent symptoms in patients with neuropathic pain (Jensen T S, The Lancet (2014); 13(9), P924-935), along with dysesthesia (an unpleasant feeling that is not actually painful per se) and paresthesia (unusual sensations, such as pins and needles or a burning sensation).

Peripheral neuropathy is associated with a range of conditions, including but not limited to the following: diabetes, amyotrophic lateral sclerosis (ALS), spinal muscular atrophy (SMA), rheumatoid arthritis, systemic lupus erythematosus (SLE), post-polio syndrome, Guillain-Barre syndrome, chronic inflammatory demyelinating polyneuropathy (CIDP), multifocal motor neuropathy, muscular dystrophy, (PML), adrenal leukodystrophy, optic neuritis, kidney disease, liver disorder, transverse myelitis, Parkinson's disease, stroke, Alzheimer's disease, Lyme disease, viral infections (e.g., an infection due to varicella-zoster virus, West Nile virus, cytomegalovirus, herpes simplex virus, or human immunodeficiency virus), carpal tunnel syndrome, peripheral nerve injuries, demyelination, acute disseminated leukoencephalitis, progressive multifocal leukoencephalitis lymphoma, neuroma, multiple myeloma, vitamin B12 deficiency, post-herpetic neuralgia, leprosy, Charcot-Marie-Tooth disease, Fabry disease, critical illness polyneuropathy, Bell's palsy, ulnar nerve palsy, and peroneal nerve palsy.

Peripheral neuropathy can also be induced by a wide range of agents, including but not limited to chemotherapeutic agents (described in detail elsewhere in this application), biologic drugs (e.g., efalizumab, infliximab, etanercept, and adalimumab), anti-alcohol drugs (e.g., disulfiram), anticonvulsants (e.g., phenytoin), heart or blood pressure medications (e.g., amiodarone), antibiotics (e.g., metronidazole and fluoroquinolones (e.g., levofloxacin, and ciprofloxacin)), skin medications (e.g., dapsone) and radiation.

The term “reducing neuropathy” in the context of this disclosure means to alleviate, ameliorate, or eliminate one or more symptoms associated with neuropathy (for example, peripheral neuropathy), including but not limited to neuropathic pain, hyperalgesia, and/or allodynia. A reduction in neuropathic symptoms can be achieved by administering the presently disclosed SapC-phospholipid nanovesicle formulations to the patient.

The administration protocols to treat neuropathy can vary based on the type of neuropathy experienced by the subject and how quickly the symptoms resolve upon treatment. In some embodiments, relatively long-term administration protocols will be needed to treat a subject with chronic neuropathy. Thus, in some embodiments, the nanovesicle formulation will be administered by any acceptable route disclosed elsewhere in this application for at least 24 hours, or discrete doses of the nanovesicle formulation will be administered periodically, e.g., for a few days, weeks or months up to a few years, as needed to reduce neuropathy and/or treat or reduce symptoms associated with neuropathy.

In some embodiments, treatment with the disclosed SapC-phospholipid nanovesicle formulation reduces the incidence or occurrence of neuropathic symptoms in a subject, compared to the incidence or occurrence of neuropathic symptoms in a control population (i.e., a group of subjects that did not receive the SapC-phospholipid treatment).

The neuropathic symptom can be experienced by the patient as a single constant event or as a series of discrete occurrences or episodes. For example, in some embodiments, a subject receiving a chemotherapeutic agent associated with neuropathic side-effects can experience the onset of one or more neuropathic episodes during or after treatment with the chemotherapeutic agent.

The SapC-phospholipid nanovesicle formulation can reduce the frequency, duration and/or intensity of the neuropathic symptom(s) experienced by the subject, and/or delay onset of neuropathy in the subject.

In some embodiments, treatment with the disclosed SapC-phospholipid nanovesicle formulation can reduce the intensity of the neuropathic symptom in a subject, compared to the intensity of the neuropathic symptom experienced by the subject prior to treatment. The intensity of a neuropathic symptom may be measured subjectively or objectively using one or more assessment tools known to those of skill in the art (e.g., patient self-reporting on the CIPN20 questionnaire). In one example, the change in the intensity of a neuropathy symptom (e.g., a symptom or groups of symptoms of CIPN) is determined by serially measuring the total neuropathy “score” obtained from one or more individual questions on a patient survey (e.g., one or more questions related to numbness, tingling, and pain on the EORTC QLQ-CIPN20 questionnaire) that quantify the intensity of that symptom as a score. The total neuropathy score obtained is then transformed to a scale of 0-100. Any change in the neuropathy score (e.g., an improvement or worsening in the score) can then be determined by comparing scores taken at various time-points during the course of treatment (e.g., before, during, or after treatment with the SapC-phospholipid formulation). Depending on how the question is worded, a reduction in intensity of the neuropathic symptom (i.e., an improvement) might be reflected as either an increase or decrease in the score. Other ways of evaluating an improvement in neuropathy scores are known in the art and described elsewhere in this application. In some embodiments, treatment with the SapC-phospholipid formulation can improve the neuropathic symptom score (e.g., reduce the intensity of an episode of a neuropathic symptom) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99%.

In some embodiments, treatment with the disclosed SapC-phospholipid nanovesicle formulation can reduce the duration of a neuropathic symptom in a subject, compared to the duration of the neuropathic symptom experienced in an appropriate control population. For example, a patient who was administered the SapC-phospholipid formulation concurrently with a chemotherapeutic agent (i.e., an agent associated with neuropathic symptom side-effects) may experience a neuropathic symptom over a shorter period of time after commencing chemotherapeutic treatment, when compared with the average duration of the neuropathic symptom experienced in the control population (i.e., a population of patients that was administered the chemotherapeutic agent but not the SapC-phospholipid formulation).

In some embodiments, treatment with the disclosed SapC-phospholipid nanovesicle formulation can reduce the duration or frequency of a neuropathic episode in a subject compared to the duration or frequency of similar neuropathic episodes experienced by the subject prior to commencing treatment with the SapC-phospholipid nanovesicle formulation. For example, prior to treatment, a patient may have periodically experienced episodes of neuropathic pain that each lasted for weeks. After treatment, the duration of each episode experienced by the patient may be reduced to a week or less, or the frequency of the episodes may be decreased, with a longer symptom-free period of time between episodes.

In some embodiments, treatment with the SapC-phospholipid formulation can reduce the duration and/or frequency of episodes of a neuropathic symptom by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 97%, or at least about 99%, as assessed by patient self-reports described elsewhere in this disclosure.

In some embodiments, treatment with the SapC-phospholipid formulation reduces the intensity of neuropathy (e.g., CIPN) on the NCI-CTC tool (Kaplow, R (2017) Nursing: 47(2)—p 67-68) to a toxicity grade of 1 or less.

### Cancer

The term “cancer” refers to the physiological condition in mammals that is typically characterized by unregulated cell growth/proliferation, and includes all types of cancer, neoplasm or malignant tumors found in mammals (e.g., humans), including leukemia (such as acute or chronic leukemia), carcinomas and sarcomas. In some embodiments, cancers that are treatable in the methods described herein include, for example, any solid tumors or neurological cancer, e.g., prostate cancer, liver cancer, lung cancer, pancreatic cancer, renal cell carcinoma, breast cancer, bladder cancer, ovarian cancer, testicular cancer, ependymoma, brain cancers such as high grade gliomas (HGG) including glioblastoma multiforme (GBM), neuroblastoma, and gastrointestinal (GI) cancers including appendiceal and colorectal cancer. The compositions are useful for treating neuropathy associated with chemotherapy treatment of metastatic tumors regardless of the primary tumor type or the organ where it metastasizes. A metastatic tumor can arise from a multitude of primary tumor types, including but not limited to those of prostate, colon, lung, breast, bone, and liver origin. “Cancer” can include malignancies of the various organ systems, such as cancers affecting the lung, breast, thyroid, lymphoid, gastrointestinal, or genito-urinary tract, as well as adenocarcinomas that include malignancies such as most colon cancers, renal-cell carcinoma, prostate cancer and/or testicular tumors, non-small cell carcinoma of the lung, cancer of the small intestine, and cancer of the esophagus. Other examples of cancers that can be treated include Hodgkin's and non-Hodgkin's lymphoma, rhabdosarcoma, Ewing's sarcoma, Wilm's tumor, and multiple myeloma. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, squamous cell carcinomas, and melanomas. Exemplary carcinomas include those forming from tissue of the cervix, lung, prostate, breast, head and neck, colon, and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. Further details can be found in, for example, U.S. Pat. No. 7,834,147, which is incorporated herein by reference in its entirety.

In some embodiments, the term “cancer” includes all types of cancer, neoplasm or malignant tumors found within the gastrointestinal system in a subject. In some embodiments, cancers that are treatable in the methods described herein (e.g., a SaposinC-DOPS nanovesicle formulation with a chemotherapeutic regimen of one or more antineoplastic agents) include, for example, any gastrointestinal cancer, including but not limited to esophageal cancer, gastric cancer, anal cancer, colorectal cancer, bowel cancer, gallbladder cancer, pancreatic cancer, liver cancer, islet cell cancer, rectal cancer, small intestine cancer, gastrointestinal carcinoid tumors, or gastrointestinal stromal tumors. The compositions are useful for treating metastatic tumors regardless of the primary tumor type or the organ where it metastasizes. A metastatic gastrointestinal tumor can arise from a multitude of primary tumor types, including but not limited to those originating in the esophagus, stomach, small intestine, large intestine, rectum, anus, liver, gallbladder, and pancreas.

In other embodiments, cancers that are treatable in the methods described herein (e.g., a SaposinC-DOPS nanovesicle formulation with one or more immune checkpoint inhibitors) include, for example, B cell lymphomas (e.g., B cell chronic lymphocytic leukemia, B cell non-Hodgkin's lymphoma, cutaneous B cell lymphoma, diffuse large B cell lymphoma, primary mediastinal large B-cell lymphoma (PBMCL)), basal cell carcinoma, bladder cancer, blastoma, brain metastasis, breast cancer, Burkitt's lymphoma, carcinoma (e.g., adenocarcinoma (e.g., of the gastroesophageal junction)), cervical cancer, colon cancer, colorectal cancer (colon cancer and rectal cancer; microsatellite instability-high cancer (MSI-H)), endometrial carcinoma, esophageal cancer (e.g., esophageal squamous cell carcinoma (ESCC)), Ewing's sarcoma, fibrosarcoma, follicular lymphoma, gastric cancer, gastroesophageal junction carcinoma, gastrointestinal cancer, glioblastoma (e.g., glioblastoma multiforme), glioma, head and neck cancer (e.g., squamous cell carcinoma of the head and neck (SCCHN or HNSSC)), Hodgkin's lymphoma (classical Hodgkin lymphoma (cHL)), non-Hodgkin's lymphoma, kidney cancer (e.g., renal cell carcinoma (RCC) and Wilms' tumors), laryngeal cancer, leukemia (e.g., chronic myelocytic leukemia, hairy cell leukemia), liver cancer (e.g., hepatic or hepatocellular carcinoma (HCC); hepatoma), lung cancer (e.g., non-small cell lung cancer and small-cell lung cancer), mesothelioma (malignant pleural mesothelioma), lymphoblastic lymphoma, lymphoma, mantle cell lymphoma, metastatic brain tumor, metastatic cancer, myeloma (e.g., multiple myeloma), neuroblastoma, ocular melanoma, oropharyngeal cancer, osteosarcoma, ovarian cancer, pancreatic cancer (e.g., pancreatic ductal adenocarcinoma), prostate cancer (e.g., hormone refractory (e.g., castration resistant), metastatic, metastatic hormone refractory (e.g., castration resistant, androgen independent)), salivary gland carcinoma, sarcoma (e.g., rhabdomyosarcoma), skin cancer (e.g., melanoma (e.g., metastatic melanoma); Merkel cell carcinoma (MCC)), soft tissue sarcoma, squamous cell carcinoma, synovia sarcoma, testicular cancer, thyroid cancer, transitional cell cancer (urothelial cell cancer; urothelial carcinoma), uveal melanoma (e.g., metastatic), verrucous carcinoma, vulval cancer, and Waldenstrom macroglobulinemia. In some instances, cancer includes any gastrointestinal cancer, including but not limited to esophageal cancer, gastric cancer, anal cancer, colorectal cancer, bowel cancer, gallbladder cancer, pancreatic cancer, liver cancer, islet cell cancer, rectal cancer, small intestine cancer, gastrointestinal carcinoid tumors, or gastrointestinal stromal tumors. The compositions are useful for treating metastatic tumors regardless of the primary tumor type or the organ where it metastasizes. A metastatic gastrointestinal tumor can arise from a multitude of primary tumor types, including but not limited to those originating in the esophagus, stomach, small intestine, large intestine, rectum, anus, liver, gallbladder, and pancreas. In some instances, the cancer comprises cells that express one or more immune checkpoint biomarkers, including PD-L1 and CTLA-4. In some instances, abundance of genomic tumor aberrations in additional biomarkers (e.g., EGFR or ALK) are determined in the cells. In some instances, detection of an immune checkpoint biomarker or a genomic tumor aberrations is performed using an FDA-approved test.

As used herein, the terms “treat,” “treating,” “treatment” and variations thereof refer to partially or completely alleviating, inhibiting, ameliorating, or relieving the disease or condition (e.g., gastrointestinal cancer, head and neck cancer, or melanoma) from which the subject is suffering. “Treating cancer” means causing a partial or complete decrease in the rate of growth of a tumor, in the size of the tumor, in the rate of local or distant tumor metastasis, in the overall tumor burden in a subject, and/or any decrease in tumor survival, using the treatment methods described herein. In certain embodiments, “treat” and its variations refers to slowing the progression or reversing the progression of cancer (e.g., gastrointestinal cancer) relative to an untreated control. The response to treatment using the disclosed methods is evaluated by a number of parameters known in the art and described below.
(1) The “objective response rate” or “ORR” is an important parameter to demonstrate the efficacy of a treatment in oncology (Aykan N F, Ozath T. (2020) World J Clin Oncol. February 24; 11(2):53-73). It assesses the tumor burden (TB) after a given treatment in patients with solid tumors by determining the proportion of patients with tumor size reduction of a predefined amount and for a minimum time period. Tumors are evaluated using World Health Organization (WHO) criteria and Response Evaluation Criteria in Solid Tumors (RECIST), which are anatomic response criteria developed mainly for cytotoxic chemotherapy, and are based on anatomical imaging. ORR is measured as the total number of subjects whose best overall response (BOR) is either complete response (CR) or partial response (PR), divided by the total number of subjects in the population of interest.
(2) The “overall survival” or “OS” is the length of time from either the date of diagnosis or the start of treatment for a disease, such as cancer, that patients diagnosed with the disease are still alive. In the context of this disclosure, it is defined as the time from the date of randomization (the date of assigning a clinical trial subject to a particular treatment group) until date of death due to any cause.
(3) “Progression free survival” or “PFS” is the length of time during and after the treatment of a disease, such as cancer, that a patient lives with the disease but it does not get worse. In the context of this disclosure, it is defined as the time from the date of randomization (the date of assigning a clinical trial subject to a particular treatment group) to the date of disease progression or death.
(4) “Duration of Response” or “DoR” is the length of time that a tumor continues to respond to treatment without the cancer growing or spreading. In the context of this disclosure, it is defined as the time from documentation of a patient's positive response to treatment until disease progression.
(5) Disease control rate (DCR) is the percentage of patients with cancer who have achieved complete response (CR), partial response (PR), or stable disease (SD). In the context of this disclosure, it is defined as the proportion of participants whose best overall response (BOR) was complete response (CR), partial response (PR) and stable disease (SD) according to RECIST v1.1 (described, for example, in Eisenhauer et al., Eur J Cancer, (2009) 45(2):228-47, the disclosure of which is incorporated herein by reference in its entirety), or according to any subsequent update to that version of RECIST.

Thus, in some embodiments, treatment with the disclosed methods leads to an increase in at least one of the following parameters in the subject relative to a control population: (a) objective response rate (ORR); (b) overall survival (OS); (c) progression free survival (PFS); and (d) duration of response (DoR). For instance, treatment with the disclosed methods can increase any one or more of the ORR, OS, PFR, and/or DoR by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99%. In addition, treatment with the disclosed methods can increase the DCR in the treated population (i.e., the group of subjects treated with the disclosed methods) compared to the control population by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99%.

### Chemotherapy-Induced Peripheral Neuropathy

Chemotherapy-induced peripheral neuropathy (CIPN) is defined as somatic or autonomic signs or symptoms resulting from damage to the peripheral nervous system (PNS) or autonomic nervous system (ANS) caused by chemotherapeutic agents (Cooper C; Chemotherapy-Induced Peripheral Neuropathy, in Cooper's Fundamentals of Hand Therapy, 2020). In CIPN, a chemotherapeutic agent (an anticancer drug) could impair both sensory and motor functions. CIPN is a common and serious side effects of cancer treatment that can lead to dose reductions or early discontinuation of chemotherapy, thereby reducing the efficacy of cancer treatments. It can cause debilitating symptoms and also significantly impacts the patient's quality of life. Approximately 30 to 40 percent of cancer patients treated with chemotherapy experience CIPN.

Platinum-based anticancer agents are commonly used to treat lung, colorectal, ovarian, breast, head/neck, and genitourinary cancers. However, peripheral neuropathy is a common adverse effect of the chemotherapy, which mainly affects dorsal root ganglia (DRG) neurons. Mechanisms that underlie peripheral neuropathy have not been fully elucidated.

Oxaliplatin, a platinum-based chemotherapy drug, is commonly used for the treatment of various types of cancer. Oxaliplatin induces two distinct forms of neuropathy: a very common acute pain syndrome that is transient and appears during, or shortly after, exposure to oxaliplatin, and a dose-limiting chronic sensory neurotoxicity that is cumulative in nature and resembles characteristics of other types of chemotherapy-related neurotoxicity, such as what is caused by cisplatin and paclitaxel. CIPN is a well described clinical problem (Grothey A, et al (2011) Journal of Clinical Oncology. 29(4):421-7; Loprinzi C L, et al (2014) J Clin Oncol. 32(10):997-1005; Pachman D R, et al (2015). Journal of Clinical Oncology 33(30):3416-22; and Pachman D R, et al (2016) Supportive Care in Cancer. 24(12):5059-68).

Chemotherapeutic agents that are known to induce neuropathy include but are not limited to platinum-based agents (e.g., oxaliplatin, cisplatin, carboplatin), taxanes (e.g., paclitaxel, docetaxel, cabazitaxel), epothilones (e.g., ixabepilone), plant alkaloids (e.g., vinblastine, vincristine, vindesine, vinorelbine, vincaminol, vineridine, vinburnine, etoposide), immunomodulatory agents (e.g., thalidomide, lenalidomide, pomalidomide), proteasome inhibitors (e.g., bortezomib, carfilzomib, ixazomib), eribulin, and suramin.

Patients with CIPN often experience symmetrical symptoms, usually starting in the hands and/or feet and creeping up the arms and legs. Patients may, in addition or alternatively, experience symptoms such as pain (shooting, sharp, stabbing and/or burning pain), numbness, sensitivity to temperature, difficulty walking, difficulty with fine motor skills such as writing, buttoning and unbuttoning, packing up small objects, etc. CIPN symptoms can also include hearing loss, blurred vision and change in taste. In addition, the motor neuron dysfunction can manifest as cramps, gait disturbances, paralysis, spasms, tremors and weakness. CIPN is sometimes characterized by the development of paresthesias, dysesthesias, loss of joint and vibration sense, and loss of deep tendon reflexes. The onset of symptomatic CIPN usually leads to reduction in dose(s) of the chemotherapeutic agent and/or interruption of therapy, which can negatively impact cancer-related outcomes.

In some embodiments, the nanovesicle formulation utilized in the disclosed methods is administered concurrently with (at the same time as, or within 48 hours before or after the start of) treatment with a chemotherapeutic agent that is associated with neuropathic symptom side effects, thereby reducing the incidence, intensity, and/or duration of neuropathy, or delaying onset of neuropathy. In some embodiments, the nanovesicle formulation is administered immediately before or after administering the dose of the chemotherapeutic agent, such that the period of time between ending administration of one of these agents and beginning administration of the other is two hours or less. For example, the period of time may be 90 minutes or less, 80 minutes or less, 70 minutes or less, an hour or less, 50 minutes or less, 40 minutes or less, 30 minutes or less, 20 minutes or less, 10 minutes or less, 5 minutes or less, or within a minute. Further, one or more additional doses of the nanovesicle formulation can be administered over the course of the weeks, months or years during which the patient continues to receive chemotherapy. Each of those additional doses of the nanovesicle formulation can be timed to be concurrent with administration of some or all of the chemotherapy doses, or can be administered on a different schedule.

In other embodiments, the nanovesicle formulation utilized in the disclosed methods is administered prior to and/or after treatment with a chemotherapeutic agent that is associated with neuropathic symptom side effects, thereby reducing the incidence, intensity, and/or duration of neuropathy, or delaying onset of neuropathy. Further, the nanovesicle formulation can be administered prior to, during, and after administration of the chemotherapeutic agent.

### Neuropathy Assessment Tools

Neuropathy symptoms are evaluated by several methods known in the art. These include but are not limited to physical examination (evaluation of sensation (including provoked pain), motor function, and autonomic changes), sensory examination to determine sensory deficits to various stimuli (e.g., touch, pinprick, temperature, vibration), blood tests, imaging studies, electrodiagnostic tests, clinical tests for nerve function (such as electromyography (EMG)), nerve conduction velocity test (nerve conduction study), autonomic reflex screen, and patient self-reporting.

Methods have been developed to distinguish between various clinical components of peripheral neuropathy, to separate pain, numbness, and tingling, and to measure them on a scale of 0 to 10. National Cancer Institute's Common Toxicity Criteria score is one of the protocols for the evaluation of peripheral neuropathy. Visual Analog Scale (VAS) Score provides a complete assessment of symptoms, signs, ability aspects, and electrophysiology of the patient. Nerve conduction studies and needle electromyography help identify the neural structure, the axonal degeneration or demyelination, and the severity of axonal damage, to confirm the diagnosis of peripheral neuropathy. The neurofilament light chain (NFL) is a reliable and easily accessible biomarker of the progression and severity of CIPN. Monitoring of neurofilament levels (e.g., serum neurofilament light chain (sNFL) levels) during chemotherapy can indicate ongoing neuroaxonal injury and the severity of CIPN (Kim, S H., et al. (2020) Sci Rep 10, 7995). Other biomarkers include NGF, BDNF, ceramides, etc. Meregalli C, et al Neurosci Lett. 2021 Apr. 1; 749:135739. The European Organization for Research and Treatment of Cancer developed a questionnaire to assess the severity of neuropathy by patient self-evaluation.

Given the subjective nature of neuropathic symptoms, measurement of these symptoms relies, in part, on patient self-reporting. In some instances, neuropathic symptoms (e.g., neuropathic side-effects of CIPN) can be evaluated based on assessment tools and inventories, including patient self-report questionnaires (e.g., CIPN20 supplementing the European Organization for Research and Treatment of Cancer (EORTC) quality of life questionnaire (Postma T J, et al. (2005). Eur J Cancer. 2005 May; 41(8):1135-9; Beijers A J, et al. (2016). Support Care Cancer. 2016 June; 24(6):2411-20). Other assessment tools include but are not limited to the Chemotherapy-Induced Peripheral Neuropathy Assessment Tool (CIPNAT), Post-Oxaliplatin Symptom Survey, the National Cancer Institute-Common Toxicity Criteria (NCI-CTC), the Numeric Rating Scale (NRS), the Visual Analog Scale (VAS), the Functional Assessment of Cancer Therapy/Gynecologic Oncology Group-Neurotoxicity (FACT/GOG-Ntx), the Total Neuropathy Score (TNS) questionnaire, and the Patient-Reported Outcome Measurement Information System (PROMIS), Cold Hypersensitivity Survey, Thermal Sensitivity Test (Tofthagen C S, et al. (2011) Cancer Nurs. 2011 July-August; 34(4):E10-20; Leonard, G. D., et al. (2005) BMC Cancer 5, 116; Dy S M, et al. (2017) Agency for Healthcare Research and Quality (US); March (Comparative Effectiveness Reviews, No. 187.) Table 3; Calhoun E A, et al. (2003) Int J Gynecol Cancer. November-December; 13(6):741-8; Cheng, H. L., et al. (2020) Health Qual Life Outcomes 18, 246; Cavaletti G, et al. (2007) J Peripher Nerv Syst. September; 12(3):210-5; Askew, R. L., et al. (2016). Value Health, 19(5), 623-630, Bae, K. H., et al. (2018). Integrative medicine research, 7(1), 61-67; Ceynowa M. et al, (2015) BioMed Research International, Article ID 528356).

These assessments are administered through short-form surveys, customized paper or electronic surveys, computer adaptive testing, etc), which aim to capture a comprehensive assessment of a patient's pain experiences, based on the patient responses. The questionnaires may include scoring systems (Yang Z, et al. (2018) Cochrane Database Syst Rev. 2018 Jul. 30; 2018(7):CD010974) to further characterize the level of neuropathy experienced by a subject. Such scoring systems include, but are not limited to Clinical Neurological Examination (CNE), Michigan Neuropathy Screening Instrument (MNSI), Neuropathy Disability Score (NDS), Neuropathy Impairment Score (NIS) Neuropathy Impairment Score in the Lower Limbs (NIS-LL), Neuropathy Symptom Profile (NSP), Neuropathy Symptom Score (NSS), Toronto Clinical Scoring System (TCSS), Neuropathy Total Symptom Score—6 (NTSS—6), Neuropathy Symptom Change Score (NSC), Total Neuropathy Score (TNS), and Total Symptom Score (TSS). Scoring systems for diabetic neuropathy include the Diabetic Neuropathy Examination (DNE) and the Diabetic Neuropathy Symptom Score (DNSS).

In some instances, neuropathic pain can be measured as a score based on patient responses to questions about pain intensity on the neuropathy pain scale (NPS) (May S, Serpell M. (2009). F1000 Med Rep. 2009 Oct. 14; 1:76), which was developed for monitoring response to treatment. On this scale, 0 indicates no pain; 10 indicates the most pain imaginable.

Screening tools to evaluate identify and evaluate neuropathic pain experienced by a subject include but are not limited to the Leeds Assessment of Neuropathic Symptoms and Signs (LANSS), the Neuropathic Pain Diagnostic Questionnaire (DN4), the Neuropathic Pain Scale (NPS), Neuropathic Pain Symptom Inventory (NPSI), and Neuropathic Pain Questionnaire (NPQ).

In other instances, neuropathic symptoms of chemotherapy-induced peripheral neuropathy (CIPN) can be characterized using toxicity grading systems, which typically use a combination of clinical and paraclinical parameters and rely on the judgment of clinicians and/or nurses (Postma T J, et al. (2005) Eur J Cancer. May; 41(8):1135-9).

Several preclinical animal models of peripheral neuropathy are used in the art to evaluate the efficacy of treatments (see, e.g., Gadgil S et al, 2019). These models include, e.g., paclitaxel-, cisplatin-, and oxaliplatin-induced rodent neuropathic pain models. Such models may be used to evaluate the efficacy of proposed neuropathy treatments.

### Neurogenesis, Neuroregeneration, Neuritogenesis, and Neuroprotection

Neurogenesis is the generation of new pre-neuronal cells. Neuroregeneration is the regrowth or repair of the nervous tissue by generating new neurons, axons, and/or synapses after an injury causing damage and/or degeneration of the nervous system. Neuroprotection refers to the preservation of neuronal structure and/or function and preventing the loss of neurons. Neuritogenesis is the extension of neurites from neuronal cell bodies via neurotrophic factors, such as nerve growth factor. In some embodiments, the present disclosure provides methods for promoting neurogenesis, neurite outgrowth, neuroprotection (including preventing neuronal loss), and neuroregeneration (such as axonal regeneration) in central nervous system (CNS) and peripheral nervous system (PNS) neurons.

Neuronal degeneration is a hallmark of many acute and chronic neuropathies. One mode of axonal degeneration, termed Wallerian Degeneration (WD) is a highly destructive process in which the part of an axon distal to an injury dies. Initial abnormities can be seen as early as several hours after injury, with more visible WD apparent a day or two later (Ballin R H and Thomas P K (1969) Acta Neuropathol (Berl) 14: 237). For instance, myelin sheaths collapse and become engulfed by scavenging cells (Leonhard et al. (2002) Eur. J. Neurosci. 16: 1654).

The disclosed methods can be used to prevent or treat an acute or chronic nerve injury, or reduce symptoms associated with an acute or chronic nerve injury. Conditions requiring neurogenesis, neuritogenesis, neuroprotection and neuroregeneration, whether acute or chronic, have been disclosed, for instance, in WO2007/044928 and references cited therein. Acute trauma to peripheral nerves includes blunt trauma as well as trauma from penetrating missiles, such as bullets or other objects. Injuries from stab wounds or foreign bodies (e.g., glass, sheet metal) resulting in clean lacerations of nerves are known, as are nerve injuries stemming from bone fractures and fracture-dislocations including ulnar nerve neurapraxia and radial nerve lesions and palsies. In general, acute nerve injury often produces a long-lasting neuropathic pain, manifested as allodynia, a decrease in pain threshold and hyperplasia, and an increase in pain in response to noxious stimuli. See Colohan A R, et al. (1996) Injury to the peripheral nerves. In: Feliciano D V, Moore E E, Mattox K L. Trauma. 3rd ed. Stamford, Conn.: Appleton & Lange; 1996:853. Other types of acute nerve injuries within the scope of the present disclosure include traumatic brain injury (TBI) and acute injuries to the spinal cord and peripheral/sensory nerves, such as sports injuries involving nerve insult.

In some embodiments, administering the nanovesicle formulation in response to an acute nerve injury promotes neurogenesis, neuritogenesis, neuroprotection and neuroregeneration when the formulation is administered as soon as possible, such as within about 24 hours after the insult (e.g., within 12, 6, 3, 2, or 1 hour after the insult). Additionally, the nanovesicle formulation can be administered prophylactically (as a precautionary measure) before a medical intervention (e.g., surgery) associated with some risk of nerve damage. This would allow nerve regeneration to be favorably enhanced and shorten recovery times.

In some embodiments, the nanovesicle formulation treats, prevents, or reduces symptoms in conditions associated with chronic injury to the nervous system, by promoting neurogenesis, neuritogenesis, neuroprotection and neuroregeneration. Such conditions include but are not limited to, a neurodegenerative disorder (e.g., Parkinson's disease, Alzheimer's disease, or amyotrophic lateral sclerosis), brain damage, peripheral nerve damage, multiple sclerosis, disseminated sclerosis, chronic demyelinating neuropathies (CMT1 type), HMSN (CMT) disease type 1A and 1B, Hereditary neuropathy with liability to pressure palsy (HNPP) and other pressure palsies, Bethlem's myopathy, Limb-Girdle muscular dystrophy, Miyoshi myopathy, rhizomelic chondrodysplasia punctata, HMSN-Lom, PXE (pseudoxanthomatosis elastica), CCFDN (congenital cataract facial dysmorphism and neuropathy), Huntington's disease, Charcot-Marie-Tooth disease, Guillain-Barré syndrome (GBS), leukodystrophy, motor neuron disease, diabetic neuropathies, distal axonopathies such as those resulting from a metabolic or toxic neuronal derangement (e.g., relating to diabetes, renal failure, exposure to a drug or toxin (e.g., an anti-cancer drug), malnutrition, or alcoholism), mononeuropathies, radiculopathies (e.g., of cranial nerve VII; Facial nerve), Hansen's disease (leprosy), plexopathies such as brachial neuritis, and focal entrapment neuropathies (e.g., carpal tunnel syndrome).

In embodiments in which the therapeutic goal is to treat a chronic nerve insult, relatively long-term administration protocols will be generally preferred. Thus, in one embodiment, the nanovesicle formulation will be administered by any acceptable route disclosed elsewhere in this application for at least 24 hours, or discrete doses of the nanovesicle formulation will be administered periodically for a few days, weeks or months up to a few years as needed to promote neurogenesis, neuritogenesis, neuroprotection and/or neuroregeneration and/or treat or reduce symptoms associated with the particular indication.

### Assessment of Neurogenesis, Neuritogenesis, Neuroprotection and Neuroregeneration

Methods for monitoring an improvement in neurogenesis, neuritogenesis, neuroprotection and neuregeneration have been described and generally include various functional tests that can be conducted in human patients. Such tests typically monitor recovery of sensory and/or motor function. Examples include, but are not limited to, the Weinstein Enhanced Sensory Test (WEST), Semmes-Weinstein Monofilament Test (SWMT) and others. See WO2008/044928, Ristic S, et al. (2000) Clin Orthop Relat Res. 370:138, Sierra, A., et al (2011). Frontiers in neuroscience, 5, 47; and references cited therein for methods for detecting and monitoring neurogenesis, neuritogenesis, neuroprotection and neuroregeneration and for methods of classifying various neuronal insults. Further methods for assessing neuronal regeneration and neuronal protection include, but are not limited to, cell-based, culture-based and tissue-based assays; and neurite outgrowth assays; neurite retraction assays; stripe assays; spot assays; scratch assays; radial assays; neuronal explants; and brain and spinal cord slice cultures. See further descriptions of these assays in e.g., Al-Ali H, et al., Exp Neurol. 2017; 287(Pt 3):423-434).

Other methods to evaluate neuroregeneration and neuroprotection include shape-texture identification (STI). Imaging techniques such as magnetic resonance imaging (MRI), MRI spectroscopy, PET imaging, and computed tomography (CT) can be used to evaluate neurogenesis, neuritogenesis, neuroprotection and neuroregeneration. In addition, neurogenesis and neuroprotection can be evaluated by techniques known in the art, e.g., obtaining tissue (e.g., via surgery) and staining it with appropriate neural markers using immunohistochemistry, or using the tissue for single-cell RNA sequencing.

In one example, an appropriate dose of a nanovesicle formulation is one that can be shown to promote neuroregeneration and neuronal preservation, as measured by intraepidermal nerve fiber density (IENFD) using skin biopsy (see e.g., Myers, M. I., & Peltier, A. C. (2013). Current neurology and neuroscience reports, 13(1), 323; and Collongues N, et al., (2018). PLoS One. January 25; 13(1):e0191614) and/or obtaining a cross-section nerve fibers and counting the different types of nerves (small unmyelinated, medium, large, large myelinated). See also, Chattopadhyay M, et al (2004) Brain. April; 127(Pt 4):929-39. Appropriate dosing can be determined by methods known in the art.

### Methods of Treating Cancer

The following sections A-E pertain to the disclosed methods of treating cancer.

### A. Antineoplastic Agents

The active agent(s) of the chemotherapeutic regimen of the disclosed methods is/are antineoplastic agent(s). The term “antineoplastic agent” refers to a medication that prevents, inhibits or delays the development of a neoplasm (a tumor) and is used to treat gastrointestinal cancer. An antineoplastic agent may also be referred to as an anti-cancer agent, a chemotherapeutic agent, a chemotherapy drug, or a cytotoxic agent. Antineoplastic agents of the present disclosure include, but are not limited to, alkylating agents, antimetabolites, plant alkaloids, and miscellaneous agents, and checkpoint inhibitors. Alkylating agents include, but are not limited to platinum-containing compounds (e.g., oxaliplatin, cisplatin, carboplatin, etc), cyclophosphamide, ifosfamide, chlorambucil, busulfan and melphalan, and carmustine. Antimetabolites include, but are not limited to, methotrexate, 5-fluorourocil, capecitabine and cytosine arabinoside. Plant alkaloids include, but are not limited to, vinca alkaloids (e.g. vincristine and vinblastine), epipodophyllotoxins (e.g. etoposide and teniposide), taxanes (e.g. paclitaxel and docetaxel). Miscellaneous agents include, but are not limited to, topoisomerase I inhibitor (e.g., irinotecan). Checkpoint inhibitors include, but are not limited to, anti-PD1 and anti-PDL1 drugs. Most antineoplastic agents are delivered intravenously, although a number of agents can be administered orally (e.g., melphalan, busulfan, capecitabine). In some embodiments, the disclosed methods include the use of the antineoplastic agents oxaliplatin and 5-fluorouracil. In the context of the present disclosure and claims, the terms “chemotherapeutic,” “chemotherapy” and “antineoplastic agent” do not encompass the formulations described above that contain both a SapC polypeptide and a phospholipid.

The combination of a chemotherapeutic regimen and a saposin C polypeptide/phospholipid formulation (e.g., BXQ-350) may enhance or prolong an anti-tumor response in a subject. Further, the administration of a saposin C polypeptide/phospholipid formulation (e.g., BXQ-350) with a chemotherapeutic regimen may enhance or prolong the therapeutic effects of the chemotherapeutic regimen, enable a subject to respond to a chemotherapeutic regimen who otherwise would not respond or respond as well, reduce the toxicity of the chemotherapeutic regimen, or enable a reduction in the dose of the chemotherapeutic regimen needed to achieve a desired therapeutic effect in the patient.

In some embodiments, the combination of a chemotherapeutic regimen and a saposin C polypeptide/phospholipid formulation of this disclosure may be used in combination with a biologic treatment, e.g., an antibody, including, but not limited to, bevacizumab, cetuximab, and panitumumab. Bevacizumab is an anti-vascular endothelial growth factor (VEGF) monoclonal antibody. Bevacizumab is FDA-approved for first line treatment of mCRC in combination with 5-FU based chemotherapy (Strickler J H, Hurwitz H I. Oncologist. 2012; 17(4):513-524). Cetuximab is an anti-epidermal growth factor receptor (EGFR) chimeric antibody, and panitumumab is an anti-epidermal growth factor receptor (EGFR) monoclonal antibody. Both cetuximab and panitumumab have demonstrated clinical efficacy in patients with chemotherapy-refractory wild-type KRAS exon 2 mCRC (Taniguchi H, et al. Cancers (Basel). 2020; 12(7):1715).

### B. Immune Checkpoint Inhibitors

The present disclosure also provides methods that use immune checkpoint inhibitors in combination with a saposin C polypeptide/phospholipid formulation (e.g., BXQ-350) to treat cancer. As used herein, an “immune checkpoint inhibitor” is a molecule (e.g., a protein, a small molecule, or a molecule comprising a protein) that blocks checkpoint protein function in an immune cell, such as T cells, and in some cancer cells. When checkpoints are blocked using the inhibitors disclosed herein, T cells can kill cancer cells more effectively. Examples of checkpoint proteins found on T cells or cancer cells include PD-1/PD-L1 and CTLA-4/B7-1/B7-2. In some instances, the immune checkpoint inhibitor does not include a nanovesicle formulation comprising (i) a saposin C polypeptide comprising SEQ ID NO: 1 with zero to four amino acid insertions, substitutions, deletions, or combination thereof; and (ii) a phospholipid having a net negative charge at neutral pH.

The combination of an immune checkpoint inhibitor and a saposin C polypeptide/phospholipid formulation (e.g., BXQ-350) may enhance or prolong an anti-tumor response in a subject. Further, the administration of a saposin C polypeptide/phospholipid formulation (e.g., BXQ-350) with an immune checkpoint inhibitor may enhance or prolong the therapeutic effects of the immune checkpoint inhibitor, enable a subject to respond to an immune checkpoint inhibitor who otherwise would not respond or respond as well, reduce the toxicity of the immune checkpoint inhibitor, or enable a reduction in the dose of the immune checkpoint inhibitor needed to achieve a desired therapeutic effect in the patient.

In some instances, the immune checkpoint inhibitor is a biologic therapeutic or a small molecule. In some instances, the immune checkpoint inhibitor is a monoclonal antibody, a humanized antibody, a fully human antibody, a single-chain Fv (scFv), a fusion protein, or a combination thereof. In some instances, the immune checkpoint inhibitor is an inhibitor of programmed death receptor-1 (PD-1). The PD-1 inhibitor can be selected from the group consisting of nivolumab, pembrolizumab, pidilizumab, MEDIO680, and combinations thereof. In some instances, the immune checkpoint inhibitor is an inhibitor of programmed death-ligand 1 (PD-L1). The PD-L1 inhibitor can be selected from the group consisting of atezolizumab, BMS-936559, MEDI4736, MSB0010718C, and combinations thereof. In some instances, the immune checkpoint inhibitor is an inhibitor of cytotoxic T-lymphocyte-associated protein 4 (CTLA-4). The CTLA-4 inhibitor can be selected from the group consisting of ipilimumab, tremelimumab, and combinations thereof.

In some instances, the patient has an unresectable and/or metastatic tumor or cancer. In other instances, the combination treatment will occur following complete or partial resection, and can occur in combination with a steroid. In some instances, the combination treatment described herein is a first-line treatment, a second-line treatment, or a third-line treatment. The combination treatment can be given after detection or in the absence of certain genomic tumor aberrations such as EGFR or ALK genomic tumor aberrations. A subject receiving the treatment can have a Tumor Proportion Score (TPS) ≥1% or Combined Positive Score (CPS) i10 as determined by an FDA-approved test.

### C. Chemotherapeutic Regimen

The term “chemotherapeutic regimen” refers to a standardized plan or protocol for a cancer treatment that uses one or more antineoplastic agents. Current chemotherapy regimens apply drug treatment in cycles, with the frequency and duration of treatments limited by toxicity. A commonly used combination chemotherapy regimen, called FOLFOX, is used to treat colorectal cancer. It includes the component leucovorin calcium (folinic acid), and the antineoplastic agents 5-fluorouracil (5FU), and oxaliplatin. There are several different FOLFOX regimens, including FOLFOX-4, FOLFOX-6, modified FOLFOX-6 (mFOLFOX-6), FOLFOX-7, and modified FOLFOX-7 (mFOLFOX-7). FOLFOX regimens differ in the doses and ways in which the three drugs are given. Examples of chemotherapeutic regimens used in the treatment of gastrointestinal cancers (e.g., colorectal carcinoma) include, but are not limited to, are 5-fluorouracil (5-FU)-/capecitabine- based combination therapies, such as: FOLFOX, FOLFIRI (5-FU, folinic acid and irinotecan) and CAPOX (capecitabine and oxaliplatin) (Sonbol M B, et al (2019). JAMA Oncol; e194489. Any of these chemotherapeutic regimens may be used in the methods of this disclosure. In some embodiments, the methods of the disclosure comprise administering the chemotherapeutic regimen mFOLFOX7. The mFOLFOX7 regimen comprises sequentially administering (i) 85 mg/m² oxaliplatin infused with 200 mg/m² leucovorin calcium for about 2 hours on the first day of treatment; and (ii) 2400 mg/m² 5-fluorouracil (5-FU) for about 46 to 48 hours on days 1 and 2 of the first week of treatment.

### C.1. Oxaliplatin

The antineoplastic agent of the chemotherapeutic regimen of the present disclosure may comprise oxaliplatin, a well-known chemotherapeutic drug that has significant antineoplastic effects. It is sold under the brand name Eloxatin®, and is a cancer medication used to treat colorectal cancer. Oxaliplatin is a platinum based antineoplastic drug that exerts its cytotoxic effect mostly through DNA damage (Alcindor T, et al (2011). Curr Oncol. 2011; 18(1):18-25). Apoptosis of cancer cells can be caused by formation of DNA lesions, arrest of DNA synthesis, inhibition of RNA synthesis, and triggering of immunologic reactions. Oxaliplatin has the chemical formula C₈H₁₄N₂O₄Pt and the chemical name of cis-[(1R,2R)-1,2-cyclohexanediamine-N,N′][oxalato(2)-O,O′] platinum. It is described in U.S. Pat. No. 4,169,846, incorporated herein by reference.

### C.2. 5-Fluorouracil

The antineoplastic agent of the chemotherapeutic regimen of the present disclosure may comprise 5-fluorouracil (5-FU), an antimetabolite fluoropyrimidine analog of the nucleoside pyrimidine with antineoplastic activity. It is sold under the brand names Adrucil®, Carac®, Efudex®, Efudix®, etc and is a cancer medication used to treat several cancers, including anal cancer, colorectal cancer, colon cancer, esophageal cancer, stomach cancer, pancreatic cancer, breast cancer, and cervical cancer. It exerts its cytotoxic effect by inhibiting thymidylate synthase (TS) and incorporating its metabolites into RNA and DNA (Longley, D., et al. (2003) Nat Rev Cancer 3, 330-338). 5-FU has the chemical formula C₄H₃FN₂O² and is described in U.S. Pat. No. 4,336,381A, incorporated herein by reference.

### C.3. Leucovorin

Leucovorin (also called folinic acid, Wellcovorin®) or leucovorin calcium, is not a chemotherapy medication, but is given in conjunction with chemotherapy. It acts as a biochemical cofactor for 1-carbon transfer reactions in the synthesis of purines and pyrimidines. Leucovorin does not require the enzyme dihydrofolate reductase (DHFR) for conversion to tetrahydrofolic acid. The effects of methotrexate and other DHFR-antagonists are inhibited by leucovorin. Leucovorin can potentiate the cytotoxic effects of fluorinated pyrimidines (i.e., fluorouracil and floxuridine). After 5-FU is activated within the cell, it is accompanied by a folate cofactor, and inhibits the enzyme thymidylate synthetase, thus inhibiting pyrimidine synthesis. Leucovorin increases the folate pool, thereby increasing the binding of folate cofactor and active 5-FU with thymidylate synthetase. Leucovorin has the chemical formula C₂₀H₂₃N₇O₇ and is described in U.S. Pat. No. 4,500,711 A, incorporated herein by reference.

In some embodiments, methods of the disclosure comprise administering the chemotherapeutic regimen mFOLFOX7 in combination with bevacizumab. Bevacizumab, cetuximab, and panitumumab are included in the National Comprehensive Cancer Network guidelines for first line therapy in combination with FOLFOX for treating cancer (NCCN.org [Internet]. Guidelines for Patients®-Colon Cancer. Plymouth Meeting, PA: National Comprehensive Cancer Network; Jul. 28, 2021. Each dose of bevacizumab is administered over a period of about 30 to about 90 minutes, e.g. 45 minutes, or 60 minutes.

### D. Treatment Cycles

The SapC-phospholipid nanovesicle formulation and the chemotherapeutic regimen of the disclosure can be administered for multiple treatment cycles (e.g., 1, 2, 3, 4, 5, or 6 cycles) in the subject. The term “cycle” refers to a period of treatment followed by a period of rest (no treatment) that is repeated on a regular schedule.

The term “day 1 of week 1 of treatment” refers to the first day that treatment with the SapC-phospholipid nanovesicle formulation commences in the subject.

In one example, Cycle 1 of treatment comprises the first four weeks of treatment. The SapC-phospholipid nanovesicle formulation is administered once a day for 5 consecutive days in the first week of treatment, once every 2 days (or 3 times a week) in the second week, and approximately once every week (i.e., every 7 (+/−3) days) in the third and fourth weeks. Cycles 2-6 comprise weeks 5-24 of treatment. In cycles 2-6, the SapC-phospholipid nanovesicle formulation is administered once every 14 (+/−3) days. Meanwhile, in all cycles (1-6), starting on day 1 of week 1, the chemotherapeutic regimen is administered once every 14 (+/−3) days. Thus, a subject with cancer is administered a total of 12 infusions of the chemotherapeutic regimen over the course of 6 treatment cycles.

In another example, Cycle 1 of treatment comprises the first four weeks of treatment. The SapC-phospholipid nanovesicle formulation is administered once a day for 5 consecutive days in the first week of treatment, once every 2 days (or 3 times a week) in the second week, and approximately once every week (i.e., every 7 (+/−3) days) in the third and fourth weeks. Cycles 2-6 comprise weeks 5-24 of treatment. In cycles 2-6, the SapC-phospholipid nanovesicle formulation is administered once every 14 (+/−3) days. Meanwhile, in all cycles (1-6), starting on day 1 of week 1, the chemotherapeutic regimen and the bevacizumab are administered once every 14 (+/−3) days. Thus, a subject with cancer is administered a total of 12 infusions of the chemotherapeutic regimen over the course of 6 treatment cycles.

In some embodiments, the treatment can be extended over a period of time. After the completion of cycles 1-6, the subject can undergo further treatment. Cycles 7-26 comprise weeks 25-104 of treatment. In Cycles 7-26, the SapC-phospholipid nanovesicle formulation is administered once every 28 (+/−3) days. Meanwhile, the chemotherapeutic regimen and/or the bevacizumab treatment continue as maintenance therapy per attending physician discretion and/or standard of care.

As used herein, the term “simultaneous administration” refers to administration of a first treatment, such as administration of a first pharmaceutical composition (e.g., a nanovesicle formulation comprising SapC-phospholipid), administration of a second treatment, such as administration of a second pharmaceutical composition (e.g., an antineoplastic agent of the chemotherapeutic regimen, or an immune checkpoint inhibitor), and administration of a third treatment, such as administration of a third pharmaceutical composition (e.g., a biologic such as bevacizumab), wherein the first, second, and third treatments are separate and are administered at around the same time, i.e., the first, second, and third pharmaceutical compositions are administered within 48 hours of each other.

In one example, the second pharmaceutical composition (i.e., at least one antineoplastic agent of the chemotherapeutic regimen or at least one immune checkpoint inhibitor) is administered soon after the SapC-phospholipid nanovesicle formulation is administered, e.g., dosing of oxaliplatin in the mFOLFOX7 chemotherapeutic regimen or dosing of an immune checkpoint inhibitor may begin at least 1 hour after completion of dosing of the SapC-phospholipid nanovesicle formulation. In another example, administration of a dose of the antineoplastic agent of the chemotherapeutic regimen (e.g., oxaliplatin) or the immune checkpoint inhibitor begins within 48 hours (e.g., within 24 hours, or within 12 hours, or within 6 hours, or within 2 hours, or 1 hour or less) after completing administration of a dose of the SapC-phospholipid nanovesicle formulation. In another example, administration of a dose of the SapC-phospholipid nanovesicle formulation begins within 48 hours (e.g., within 24 hours, or within 12 hours, or within 6 hours, or within 2 hours, or 1 hour or less) after completing administration of a dose of the antineoplastic agent or the immune checkpoint inhibitor.

In some cases, the second pharmaceutical composition (i.e., at least one antineoplastic agent of the chemotherapeutic regimen) and the third pharmaceutical composition (e.g., bevacizumab) are administered simultaneously, such that their administration overlaps. In one example, the at least one antineoplastic agent (e.g., 5-FU) is administered intravenously over several minutes, and this administration continues via a pump (e.g., an ambulatory infusion pump) that continuously delivers 5-FU over a period of 46 to 48 hours. During the process of 5-FU infusion, bevacizumab is administered over a period of 30 to 90 minutes. Alternatively, bevacizumab is administered before (e.g., an hour before) 5-FU infusion is begun, or after (e.g., an hour after) 5-FU infusion is completed.

In yet another example, the SapC-phospholipid nanovesicle formulation is first administered in doses given on multiple consecutive days for a period of time (e.g., once a day on 3, 4, 5, or 6 consecutive days in the first week). Thereafter, the frequency of administering the SapC-phospholipid formulation in weeks 2-4 is reduced (e.g., three times a week, or once a week). From week 5 through 6 months, the SapC-phospholipid formulation is administered once every 14 (+/−3) days. From 6 months through 2 years, the SapC-phospholipid formulation is administered once every 28 (+/−3) days. Meanwhile, the chemotherapeutic regimen or immune checkpoint inhibitor, and optionally the bevacizumab, are administered according to standard protocols, e.g., once every 14 (+/−3) days, starting within 48 hours (e.g., within 24 hours, or within 12 hours, or within 6 hours, or within 2 hours, or 1 hour or less) after completing administration of the last consecutive dose of the SapC-phospholipid nanovesicle formulation, and continuing through 6 months. The chemotherapeutic regimen or immune checkpoint inhibitor, and optionally the bevacizumab therapy, may be continued for up to 2 years as maintenance therapy, per attending physician discretion or standard of care. In a further example, the chemotherapeutic regimen or immune checkpoint inhibitor regimen, and optionally the bevacizumab treatment, may start long after beginning the SapC-phospholipid regimen, e.g. in week 2, 3, 4, 5, or 6.

As one example, the SapC-phospholipid nanovesicle formulation is administered once a day for 5 consecutive days in the first week of treatment, once every 2 days (or 3 times a week) in the second week, and approximately once every week (i.e., every 7 (+/−3) days) in the third and fourth weeks. Thereafter, the SapC-phospholipid nanovesicle formulation is administered once every 14 (+/−3) days, or alternatively once every 7 (+/−3 days), or alternatively once every 21 (+/−3 days), or alternatively once every 28 (+/−3 days). The chemotherapeutic regimen or immune checkpoint inhibitor regimen is administered once every 14 (+/−3) days. Bevacizumab is administered once every 14 (+/−3) days or once every 21 (+/−3 days).

In an alternate example, the SapC-phospholipid nanovesicle formulation is administered as in the examples above after completing a standard chemotherapeutic regimen (optionally including a biologic such as bevacizumab) or immune checkpoint inhibitor regimen. In yet another example, the SapC-phospholipid nanovesicle formulation administration is started during the chemotherapeutic regimen (and optionally with a biologic (e.g., bevacizumab) administration) or immune checkpoint inhibitor regimen.

Each dose of the SapC-phospholipid nanovesicle formulation is administered over a period of about 45 minutes to about 120 minutes (e.g., about 45 minutes, about 60 minutes, or about 90 minutes).

The active ingredients for the first, second, and third pharmaceutical compositions (i.e., the nanovesicle formulation comprising SapC-phospholipid, the one or more antineoplastic agents of the chemotherapeutic regimen or the one or more immune checkpoint inhibitors and/or the biologic) typically are not present in the same composition.

In another example, treatment with a SapC-phospholipid nanovesicle formulation and an immune checkpoint inhibitor is indicated for subjects who have not responded to other treatments, such as steroids, radiotherapy, or chemotherapy. In some instances, the treatment involves front-loading with the SapC-phospholipid nanovesicle formulation prior to treatment with an immune checkpoint inhibitor.

### E. Therapeutically Effective Dose for Treating Cancer

As described above, the methods of treating cancer (e.g., gastrointestinal cancer) provided herein comprise administering a nanovesicle formulation having as an active agent SapC-DOPS and a chemotherapeutic regimen having one or more antineoplastic agents, to a subject in need thereof. Each agent is administered in an amount (i.e., a “therapeutically effective amount”) and for such time as is necessary to achieve the desired result.

In the context of treating cancer, a “therapeutically effective amount” or “therapeutically effective dose” of the nanovesicle formulation or of the antineoplastic agent is an amount useful to treat a patient's cancer, i.e., the amount of the agent that modulates or ameliorates the symptoms or condition of a cancer, tumor, or neoplastic disease, e.g., prevents or reduces viability of the cancer cells. It can include a single treatment or a series of treatments.

The therapeutic dose of the compositions of the present disclosure (i.e., the nanovesicle formulation with SapC-phospholipid and/or the antineoplastic agents of the chemotherapeutic regimen) may be decided by the attending physician within the scope of sound medical judgment and experience. For the active agent, the therapeutically effective dose may be estimated initially in cell culture assays or in animal models such as mice, rats, rabbits, dogs, pigs, or monkeys. Animal models may be used to achieve or determine a desirable concentration and total dosing range and route of administration, which may be used to determine a useful range of dosage and routes for administration in humans. Further, clinical studies and individual patient response may determine the recommended therapeutic dose.

In general, a single therapeutically effective dose of the nanovesicle formulation will contain an amount of SapC (or its derivative) in the range of about 0.01 to 30 mg/kg body weight, preferably about 0.05 to 20 mg/kg body weight, more preferably about 0.1 to 15 mg/kg body weight, and even more preferably about 0.5 to 10 mg/kg. For example, the amount of SapC in a single intravenous dose can be about 0.4 mg/kg, 0.7 mg/kg, 1.1 mg/kg, 1.4 mg/kg, 1.8 mg/kg, 2.4 mg/kg, 2.8 mg/kg, 3.0 mg/kg, 3.2 mg/kg, 3.6 mg/kg, 7 mg/kg or more. A given patient may receive a given dose level for one or more initial administrations and a different (lower or higher) level for further administrations.

A therapeutic dose of the antineoplastic agent oxaliplatin is in the range of 50-150 mg/m², such as 85 mg/m². A therapeutic dose of the antineoplastic agent 5-fluorouracil is in the range of 500-2400 mg/m², such as 2400 mg/m². A therapeutic dose of the antineoplastic agent paclitaxel is in the range of 90 mg/m² to 250 mg/m², such as 125 mg/m² to 175 mg/m². A therapeutic dose of the bevacizumab is in the range of 5-15 mg/kg, such as 5 mg/kg, 7.5 mg/kg, 10 mg/kg, or 15 mg/kg. The 5 mg/kg, 7.5 mg/kg and 10 mg/kg doses are generally administered every two weeks. The 15 mg/kg doses are generally administered every three weeks.

The results of treatment with the methods of the present disclosure may be evaluated or determined by any method known to those skilled in the art, including, but not limited to, imaging, ultrasound, physical examination, or blood tests.

As used herein, “pharmaceutical composition” refers to any chemical or biological composition, material, agent or the like that is capable of inducing a therapeutic effect when properly administered to a subject, including the composition, material, agent or the like in an inactive form and active metabolites thereof, where such active metabolites may be formed in vivo.

The results of treatment with the methods of the present disclosure may be evaluated or determined by any method known to those skilled in the art, including, but not limited to, imaging, ultrasounds, physical examination, blood tests, and radioimmunoassays.

### Kits

The pharmaceutical compositions described herein may be included in a kit, pack, or dispenser, referred to collectively herein as a “kit,” optionally with instructions for administration of such pharmaceutical compositions. In some embodiments, a kit may include a first pharmaceutical composition comprising a saposin C-phospholipid formulation (e.g., in solid form, such as in the form of a lyophilized powder) and a second pharmaceutical composition comprising at least one immune checkpoint inhibitor (e.g., a PD-1 inhibitor selected from nivolumab, pembrolizumab, pidilizumab, or MEDIO680; a PD-L1 inhibitor selected from atezolizumab, BMS-936559, MEDI4736, or MSB0010718C; or a CTLA-4 inhibitor selected from ipilimumab or tremelimumab).

In some embodiments, a kit may include, in separate containers, a first pharmaceutical composition, i.e., the nanovesicle formulation comprising SapC-phospholipid, and a second pharmaceutical composition comprising at least one antineoplastic agent (e.g., oxaliplatin or 5-FU), and may further include a third container with a third composition that may be, e.g., another antineoplastic agent, e.g., oxaliplatin or 5-FU. The kit may also include a container with a composition comprising a biologic, e.g., bevacizumab.

Optionally, additional pharmaceutical compositions also may be included in the kit. The kit also may include one or more containers of a pharmaceutically acceptable carrier (e.g., sterile water or saline) suitable for reconstituting or diluting each pharmaceutical composition included in the kit. The compositions and carrier(s) may be housed in vials or other suitable containers such as syringes. Typically, the first and second pharmaceutical compositions will be in separate containers in the kit. Each composition may be in the form of a dry powder, a liquid, a suspension in a liquid, frozen, or in any other suitable form.

It is to be understood that the disclosed methods and compositions may assume various alternative variations and step sequences, except where expressly specified to the contrary. Moreover, other than in any operating examples, or where otherwise indicated, all numbers such as those expressing values, amounts, percentages, ranges, subranges and fractions may be read as if prefaced by the word “about”, even if the term does not expressly appear. Accordingly, unless indicated to the contrary, the numerical parameters set forth in the specification and attached claims are approximations that may vary depending upon the results desired to be obtained by the disclosed methods. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

As used herein, unless indicated otherwise, a plural term can encompass its singular counterpart and vice versa, unless indicated otherwise. For example, although reference is made herein to “a” composition, a combination (i.e., a plurality) of these components can be used. In addition, in this application, the use of “or” means “and/or” unless specifically stated otherwise, even though “and/or” may be explicitly used in certain instances.

As used herein, “including”, “containing” and like terms are understood in the context of this application to be synonymous with “comprising” and are therefore open-ended and do not exclude the presence of additional undescribed and/or unrecited elements, materials, ingredients and/or method steps.

As used herein, “consisting of” is understood in the context of this application to exclude the presence of any unspecified element, ingredient and/or method step.

As used herein, “consisting essentially of” is understood in the context of this application to include the specified elements, materials, ingredients and/or method steps and others that are unspecified, provided that the latter “do not materially affect the basic and novel characteristic(s)” of what is being described.

The compositions and methods are further supported by the information provided in the following Examples. It is to be understood that the embodiments described in the Examples are merely illustrative, and are not intended to limit the scope of the present disclosure, which will be limited only by the appended claims.

### EXAMPLES

### Example 1. SapC-DOPS (BXQ-350) Improves Neuropathy in Subjects Diagnosed with Cancer and/or Diabetes

A Phase 1 study to determine the anticancer effectiveness of BXQ-350 was conducted in adult patients with advanced solid tumors (Clinical Trials.gov Identifier NCT02859857) who had been previously treated with chemotherapeutic agents. Each participant received multiple doses of BXQ-350 at the 2.4 mg/kg dose level. Unexpectedly, some patients in the study experienced relief from their ongoing neuropathy symptoms. These patients are described below.

Patient 1: The patient is a 70 year old woman with a history of metastatic pancreatic cancer. The patient completed five (5) cycles of FOLFIRINOX (folinic acid/fluorouracil/irinotecan/oxaliplatin) with good response. Oxaliplatin was discontinued due to grade 2 peripheral sensory neuropathy. One additional cycle of FOLFIRI was completed, followed by gemcitabine and radiation therapy for 3 weeks. Two years later, the patient developed metastasis to the lungs from a known primary pancreatic cancer. The patient was treated with palliative gemcitabine and nabpaclitaxel for about two additional years (22 months) with persistent grade 1 peripheral sensory neuropathy before disease progression. The patient enrolled in the BXQ-350 clinical trial. After 2 cycles of BXQ-350 at 2.4 mg/kg administered intravenously, the patient reported that the grade 1 peripheral sensory neuropathy that had been present since she was treated with oxaliplatin 5 years prior was completely resolved, along with red color on her feet that flared with neuropathic pain. The patient's neuropathy improved to the point that, after disease progression, FOLFOX could be restarted.

Patient 2: A metastatic colorectal cancer patient with persistent neuropathy related to prior treatment for many years reported resolution of neuropathy symptoms while being treated with BXQ-350 at 2.4 mg/kg administered intravenously.

Patient 3: A metastatic parotid/salivary gland cancer patient with persistent residual peripheral sensory neuropathy in hands and feet from prior treatments with cisplatin/navelbine and paclitaxel, and currently treated with gabapentin, noted resolution of numbness and tingling in hands and some improvement in feet with persistence of numbness after treatment with BXQ-350 at 2.4 mg/kg administered intravenously.

Patient 4: A patient with esophageal cancer and a medical history of peripheral neuropathy in both feet was treated with BXQ-350 at 2.4 mg/kg administered intravenously. The patient reported mild improvement on CIPN questionnaires: tingling in toes/feet improved from 3 to 2 on a scale of 1-4 (with 1 indicating no tingling and 4 indicating severe tingling) at day 113 from start of BXQ-350 treatment; numbness in toes/feet improved from 3 at day 1 to 2 for the remaining visits; difficulty feeling the ground under your feet improved from 2 at day 1 to 1 for the remainder of the visits; difficulty walking because your foot dropped downward improved from 3 at day 1, 2 at days 57 and 85, and 1 at day 113.

Patient 5: A pancreatic cancer patient with persistent Grade 1 platinum induced peripheral neuropathy noted improvement during treatment with BXQ-350.

Patient 6: A pancreatic cancer patient with neuropathy resulting from FOLFOX treatment (last dose in November 2018) noted improvement in neuropathy after beginning treatment with BXQ-350 in March 2019.

Patient 7: A metastatic colon cancer patient with grade 1 peripheral neuropathy associated with past treatment with FOLFOX (treatment ended in October 2018) began treatment with BXQ-350 on Feb. 18, 2019. The patient subjectively noted improvement in the neuropathy symptoms while receiving BXQ-350.

Out of 10 patients in the trial who had chemotherapy-induced peripheral neuropathy (CIPN) symptoms and who were questioned and evaluated regarding neuropathy symptoms: 4 patients (Patients 1-4) with long term peripheral neuropathy noted resolution or improvement of symptoms; 3 patients (Patients 5-7) noted resolution of symptoms, but, due to timing between treatment with chemotherapy and treatment with BXQ-350, it was unclear if this was due to stopping chemotherapy, e.g. washout, or a beneficial effect of BXQ-350; and 3 patients reported no changes to their pre-existing neuropathic symptoms while being treated with BXQ-350. Persistent CIPN typically does not improve years later, so 4 out of 10 patients clearly reporting resolution/improvement of persistent peripheral neuropathy years after ending chemotherapy was surprising and unexpected.

In addition, the trial had a patient with a gastrointestinal stromal tumor (GIST). He also had long-term diabetic neuropathy. After starting BXQ-350 at 2.4 mg/kg administered intravenously, the patient noted that his symptoms of intermittent tingling in the fingers became subjectively much less frequent.

### Example 2. SapC-DOPS (BXQ-350) Prevents Neuropathic Symptoms in a Mouse Model of Chemotherapy (Oxaliplatin) Induced Peripheral Neuropathy (CIPN)

BXQ-350 was tested in a mouse oxaliplatin model of chemotherapy-induced neuropathy. All experiments were performed according to the Ethical Guidelines of the International Association for the Study of Pain (Zimmerman, 1983). All behavioral tests were approved by the United Kingdom Home Office Animals (Scientific Procedures) Act 1986.

### Background

Chemotherapy-induced neuropathy (CIN) is a significant side effect of various chemotherapeutic drugs. Oxaliplatin, a platinum-based chemotherapy drug, is commonly used for the treatment of various types of cancer and is known to cause acute and chronic forms of peripheral neuropathy in rodents (Ling et al., 2007). Cold hypersensitivity, considered the hallmark of oxaliplatin-induced neuropathy, can be characterized as abnormal cold triggered sensations, localized to hands and feet (Attal et al., 2009). This cold hypersensitivity can also be seen in rodents after a single intraperitoneal injection of oxaliplatin (e.g. Ling et al., 2007, Zhao et al., 2012).

### Study 1

### Animals

60 adult male C57/BL6 mice (Charles River), 6-7 weeks of age, were housed in groups of 5 in Perspex cages, in a controlled environment of constant temperature and humidity (temperature: 21±1° C., light: dark cycle of 12:12 hours), with food and water available ad libitum. Animals were allowed to recover from transportation for at least one week before commencing experiments. The mice were assigned to the various groups shown in Table 2.

TABLE 2
Animal groups

| | | | | | Dose | |
|---|---|---|---|---|---|---|
| Group | CIN treatment | Dose Level | Days of Test | | Level and | No. of |
| ID | (Day 0) | and route | Article Dose | Test Article | route | Animals |
| A | Vehicle | 10 mL/kg IP | −3, −2, −1 and 1 to 9 | Vehicle | 10 mL/kg IP | 12 |
| B | Oxaliplatin | 10 mg/kg IP | −3, −2, −1 and 1 to 9 | Vehicle | 10 mL/kg IP | 12 |
| C | Vehicle | 10 mL/kg IP | −3, −2, −1 and 1 to 9 | BXQ-350 | 20 mg/kg IP | 12 |
| D | Oxaliplatin | 10 mg/kg IP | −3, −2, −1 and 1 to 9 | BXQ-350 | 20 mg/kg IP | 12 |
| E | Oxaliplatin | 10 mg/kg IP | 3, 5, 7, and 9 | Pregabalin | 30 mg/kg IP | 12 |

CIN: chemotherapy-induced neuropathy

### Experimental Design

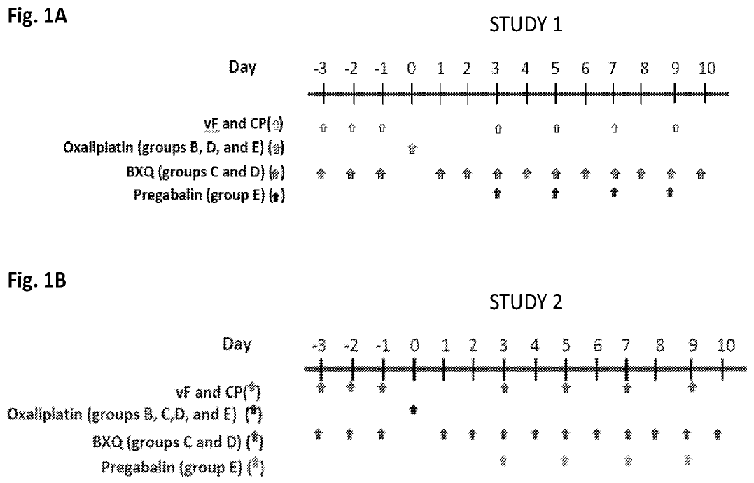

As shown in Table 2, mice (n=12 per group) in groups A-E were administered 10 mg/kg intraperitoneal oxaliplatin or vehicle at day 0, and administered test article as follows: 10 mg/kg vehicle or 20 mg/kg BXQ-350 on days −3, −2, −1, and 1 to 9; or 30 mg/kg pregabalin on days 3, 5, 7, and 9. FIG. 1A shows the timeline of administration of the various treatments to mice in groups A-E.

### Apparatus and Evaluation of Study 1

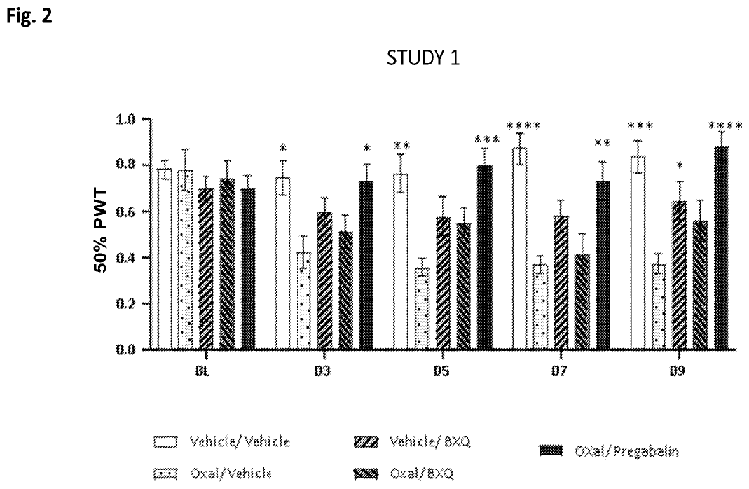

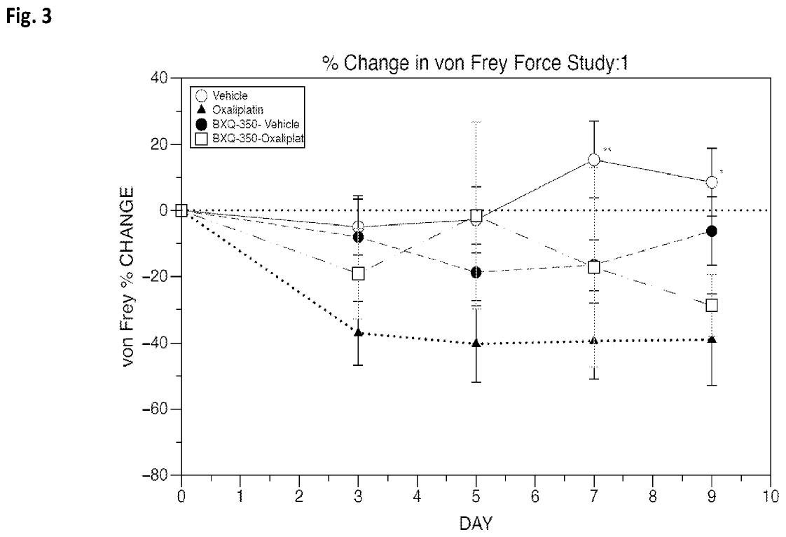

Static mechanical tactile allodynia: Static mechanical (tactile) allodynia was assessed by measurement of withdrawal threshold using calibrated (force; g) von-Frey monofilaments (Touch-Test Sensory Evaluator; Scientific Marketing Associates) applied to the plantar surface of the left hind-paw. The animals were placed in individual Perspex boxes on a raised metal mesh for 30-40 min before the test to habituate. A series of graduated von Frey hairs (0.07, 0.16, 0.4, 0.6 and 1 g) were applied in sequence with a protocol of 1 sec on 1 sec off repeated 10 times. Each hair was applied perpendicularly to the center of the ventral surface of the paw until it bent slightly. The force applied to the hind-paw of the animal to induce 5 responses out of 10 trials was recorded as paw withdrawal threshold (PWT) 50%. The results of the study are shown in Table 3 (raw data), Table 4 (summary), FIG. 2, and FIG. 3.

TABLE 3
Individual Paw Withdrawal Threshold
50% (von Frey filaments) values (g)
Group A - Vehicle/Vehicle

| ID | 1 | 2 | 3 | 4 | 5 | 6 | 8 | 9 | 10 | 11 | 12 |
|---|---|---|---|---|---|---|---|---|---|---|---|
| BL | 1.0 | 1.0 | 0.7 | 0.6 | 0.8 | 0.8 | 0.8 | 0.8 | 0.7 | 0.6 | 0.8 |
| D 3 | 1.0 | 1.0 | 0.6 | 0.4 | 0.6 | 1.0 | 0.6 | 0.4 | 1.0 | 0.6 | 1.0 |
| D 5 | 1.0 | 1.0 | 1.0 | 0.4 | 0.6 | 1.0 | 0.6 | 0.4 | 1.0 | 0.4 | 1.0 |
| D 7 | 1.0 | 1.0 | 1.0 | 1.0 | 1.0 | 0.6 | 0.4 | 0.6 | 1.0 | 1.0 | 1.0 |
| D 9 | 1.0 | 1.0 | 1.0 | 0.4 | 0.6 | 1.0 | 0.6 | 0.6 | 1.0 | 1.0 | 1.0 |

Group B - Oxaliplatin/Vehicle

| ID | 13 | 14 | 15 | 16 | 17 | 18 | 19 | 20 | 21 | 22 | 23 | 24 |
|---|---|---|---|---|---|---|---|---|---|---|---|---|
| BL | 0.3 | 0.5 | 1.0 | 0.5 | 1.0 | 0.3 | 0.8 | 1.0 | 1.0 | 1.0 | 1.0 | 1.0 |
| D 3 | 0.2 | 0.4 | 0.4 | 0.4 | 0.6 | 0.4 | 0.6 | 1.0 | 0.4 | 0.4 | 0.2 | 0.2 |
| D 5 | 0.4 | 0.4 | 0.2 | 0.4 | 0.2 | 0.4 | 0.4 | 0.4 | 0.4 | 0.6 | 0.2 | 0.4 |
| D 7 | 0.4 | 0.4 | 0.6 | 0.4 | 0.2 | 0.4 | 0.4 | 0.4 | 0.4 | 0.4 | 0.1 | 0.4 |
| D 9 | 0.2 | 0.4 | 0.4 | 0.4 | 0.4 | 0.6 | 0.2 | 0.4 | 0.4 | 0.4 | 0.6 | 0.2 |

Group C - BXQ-350/Vehicle

| ID | 25 | 26 | 27 | 28 | 29 | 30 | 31 | 32 | 33 | 34 | 35 | 36 |
|---|---|---|---|---|---|---|---|---|---|---|---|---|
| BL | 0.8 | 1.0 | 1.0 | 0.5 | 0.8 | 0.8 | 0.6 | 0.6 | 0.5 | 0.5 | 0.6 | 0.7 |
| D 3 | 0.6 | 0.4 | 1.0 | 0.6 | 0.4 | 0.6 | 0.6 | 0.4 | 0.6 | 1.0 | 0.6 | 0.4 |
| D 5 | 0.4 | 1.0 | 1.0 | 0.2 | 1.0 | 0.6 | 0.2 | 0.6 | 0.6 | 0.4 | 0.4 | 0.6 |
| D 7 | 0.6 | 0.6 | 1.0 | 0.6 | 1.0 | 0.4 | 0.6 | 0.6 | 0.2 | 0.4 | 0.4 | 0.6 |
| D 9 | 0.4 | 1.0 | 1.0 | 0.4 | 1.0 | 0.6 | 0.6 | 1.0 | 0.4 | 0.6 | 0.6 | 0.2 |

Group D - BXQ-350/Oxaliplatin

| ID | 37 | 38 | 39 | 40 | 41 | 42 | 43 | 44 | 45 | 46 | 47 | 48 |
|---|---|---|---|---|---|---|---|---|---|---|---|---|
| BL | 0.6 | 1.0 | 0.8 | 0.1 | 1.0 | 0.8 | 1.0 | 0.8 | 0.7 | 0.5 | 0.6 | 1.0 |
| D 3 | 0.4 | 0.4 | 0.4 | 0.2 | 1.0 | 0.6 | 0.4 | 0.4 | 1.0 | 0.4 | 0.4 | 0.6 |
| D 5 | 0.4 | 0.6 | 0.6 | 0.4 | 0.6 | 0.4 | 1.0 | 1.0 | 0.4 | 0.4 | 0.4 | 0.4 |
| D 7 | 0.2 | 0.4 | 0.2 | 0.4 | 1.0 | 0.2 | 0.2 | 0.6 | 0.2 | 0.4 | 0.4 | 1.0 |
| D 9 | 0.4 | 1.0 | 1.0 | | 1.0 | 0.4 | 0.2 | 0.4 | 0.6 | 0.4 | 0.4 | 0.4 |

Group E - Pregabalin/Oxaliplatin

| ID | 49 | 50 | 51 | 52 | 53 | 54 | 55 | 56 | 57 | 58 | 59 | 60 |
|---|---|---|---|---|---|---|---|---|---|---|---|---|
| BL | 0.8 | 1.0 | 0.8 | 0.7 | 0.6 | 0.4 | 0.5 | 0.8 | 0.8 | 0.6 | 1.0 | 0.4 |
| D 3 | 1.0 | 0.6 | 0.6 | 0.4 | 0.4 | 0.6 | 0.6 | 1.0 | 1.0 | 1.0 | 1.0 | 0.6 |
| D 5 | 1.0 | 1.0 | 0.4 | 1.0 | 0.6 | 0.4 | 0.6 | 1.0 | 1.0 | 0.6 | 1.0 | 1.0 |
| D 7 | 1.0 | 0.6 | 0.6 | 1.0 | 0.4 | 0.4 | 0.4 | 1.0 | 1.0 | 1.0 | 1.0 | 0.4 |
| D 9 | 1.0 | 1.0 | 0.6 | 1.0 | 1.0 | 0.6 | 1.0 | 1.0 | 1.0 | 1.0 | 1.0 | 0.4 |

Table 3: Individual Paw Withdrawal Threshold 50% (von Frey filaments) values (g). The ID numbers indicate the mouse ID.
BL: Baseline; D 3, D 5, D 7, and D 9 refer to Days 3, 5, 7, and 9 respectively.

TABLE 4
Summary of results: Mechanical allodynia

| | | | PWT | PWT | PWT | PWT | PWT |
|---|---|---|---|---|---|---|---|
| | | | 50% (g) | 50% (g) | 50% (g) | 50% (g) | 50% (g) |
| Group | Treatment | N | Baseline | D3 | D5 | D7 | D9 |
| A | Vehicle/ | 11 | 0.782 ± | *0.745 ± | **0.764 ± | ****0.873 ± | ****0.836 ± |
| | Vehicle | | 0.040 | 0.077 | 0.085 | 0.068 | 0.070 |
| B | Oxaliplatin/ | 12 | 0.780 ± | 0.423 ± | 0.357 ± | 0.369 ± | 0.373 ± |
| | Vehicle | | .087 | 0.068 | 0.038 | 0.038 | 0.043 |
| C | Vehicle/ | 12 | 0.700 ± | 0.600 ± | 0.577 ± | 0.580 ± | *0.647 ± |
| | BXQ 350 | | 0.052 | 0.060 | 0.086 | 0.069 | 0.084 |
| D | Oxaliplatin/ | 12 | 0.743 ± | 0.513 ± | 0.550 ± | 0.417 ± | 0.560 ± |
| | BXQ 350 | | 0.076 | 0.073 | 0.066 | 0.089 | 0.090 |
| E | Oxaliplatin/ | 12 | 0.698 ± | *0.733 ± | ***0.800 ± | **0.733 ± | ****0.883 ± |
| | Pregabalin | | 0.060 | 0.071 | 0.074 | 0.083 | 0.063 |

Table 4: Left hind paw Paw Withdrawal Threshold 50% (g). Two-way ANOVA showed a main effect of time (F (3.931, 211.3) = 5.518; p<0.001), a main effect of treatment (F (4, 54) = 5.518; p < 0.0001) and a significant interaction between time and treatment factors (F(16, 2.2926) = 2.926.
*, **, ***, and **** indicate statistically significant difference, with p < 0.05, p < 0.01, p < 0.001 and p < 0.0001 respectively, when compared to the Oxal/Vehicle group (Group B) at the same timepoint.
Values are shown as mean ± SEM.

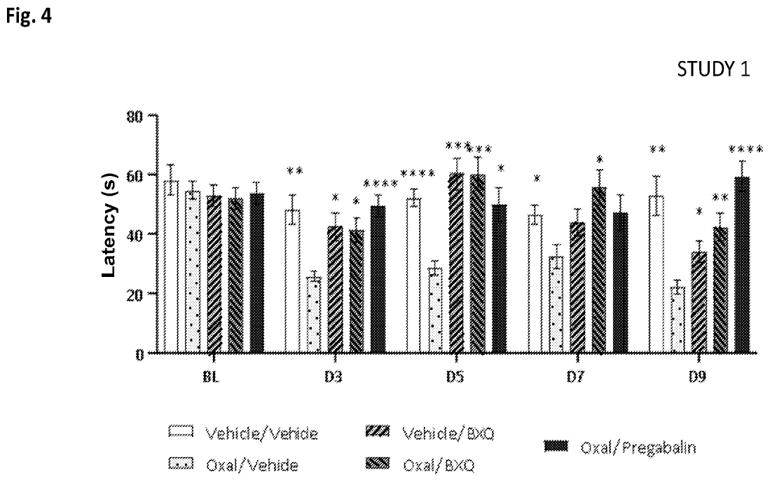

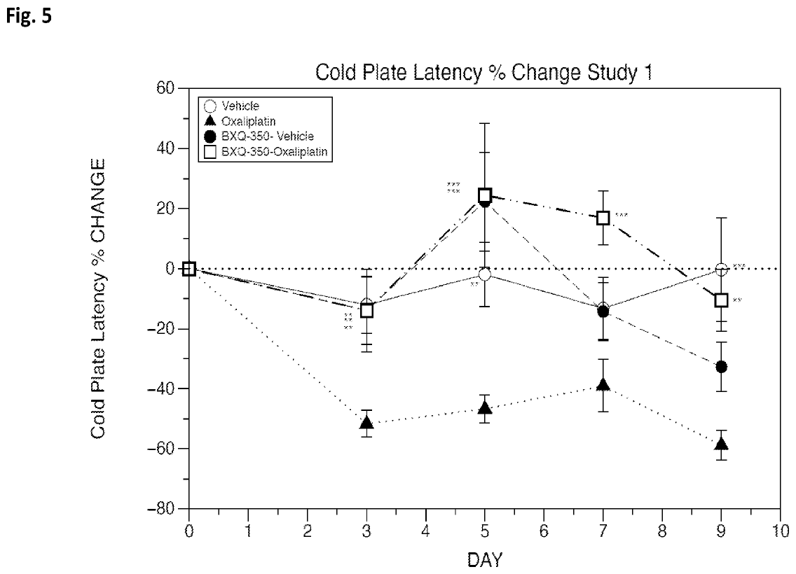

Thermal hypersensitivity: Thermal hypersensitivity was assessed using a dynamic cold plate (Bioseb, France) set at a fixed temperature of 15° C. This method allows analysis of more integrated behaviors rather than reflex behaviors, e.g., the tail immersion test. In animal studies, thermal sensitivity is mostly evaluated based on nociceptive reaction latencies in response to a given thermal aversive stimulus. In this classical test, the temperature of the plate is fixed and remains constant: the latency to the first paw licking, jumps or withdrawal is taken as an index of nociceptive threshold. Normal animals typically manifest escape behaviors at approximately 5° C., whilst animals treated with oxaliplatin present the same escape behavior at elevated temperatures (approx. 15° C.). The results of the study are shown in Table 5 (raw data) below, Table 6 (summary) below, FIG. 4, and FIG. 5.

TABLE 5
Individual latency to displaying withdrawal behaviour in the cold plate test (s)
Group A - Vehicle/Vehicle

| ID | 1 | 2 | 3 | 4 | 5 | 6 | | 9 | 10 | 11 | 12 |
|---|---|---|---|---|---|---|---|---|---|---|---|
| BL | 74.0 | 26.9 | 92. | 54.0 | 57.5 | 45.7 | 68.2 | 59.6 | 43.3 | 54.0 | 62.3 |
| D 3 | 41.1 | 28.2 | 41.8 | 35.1 | 72.3 | 53.0 | 45.7 | 84.9 | 44.3 | 40.8 | 42.7 |
| D 5 | 56.5 | 47.1 | 39.0 | 45.9 | 36.5 | 53.4 | 68.6 | 67.6 | 54.2 | 53.6 | 50.9 |
| D 7 | 47.1 | 43.5 | 59.1 | 60.5 | 38.5 | 36.0 | 54.9 | 40.3 | 58.2 | 35.6 | 37.1 |
| D 9 | 49.4 | 67.7 | 108.6 | 70.7 | 38.3 | 45.5 | 26.5 | 46.0 | 46.7 | 36.5 | 45.4 |

Group B - Oxaliplatin/Vehicle

| ID | 13 | 14 | 15 | 16 | 17 | 18 | 19 | 20 | 21 | 22 | 23 | 24 |
|---|---|---|---|---|---|---|---|---|---|---|---|---|
| BL | 45.4 | 80.0 | 54.3 | 46.2 | 46.7 | 46.5 | 58.8 | 48.6 | 57.6 | 59.0 | 48.3 | 65.5 |
| D 3 | 22.9 | 30.2 | 15.0 | 31.3 | 34.1 | 22.0 | 24.3 | 35.5 | 22.1 | 19.5 | 25.9 | 24.2 |
| D 5 | 30.3 | 31.1 | 38.3 | 24.5 | 18.5 | 16.0 | 35.3 | 30.1 | 33.9 | 27.7 | 39.8 | 17.3 |
| D 7 | 16.8 | 24.3 | 28.8 | 18.6 | 23.0 | 62.9 | 31.8 | 21.5 | 28.9 | 45.9 | 49.6 | 37.6 |
| D 9 | 12.6 | 31.8 | 20.9 | 17.4 | 25.0 | 39.6 | 10.6 | 19.4 | 25.8 | 13.6 | 24.8 | 23.1 |

Group C - BXQ-350/Vehicle

| ID | 25 | 26 | 27 | 28 | 29 | 30 | 31 | 32 | 33 | 34 | 35 | 36 |
|---|---|---|---|---|---|---|---|---|---|---|---|---|
| BL | 55.2 | 55.2 | 50.8 | 77.4 | 69.8 | 33.8 | 57.8 | 54.0 | 43.1 | 37.3 | 53.8 | 46.6 |
| D 3 | 49.0 | 22.7 | 73.6 | 22.7 | 23.5 | 29.6 | 60.4 | 42.3 | 53.6 | 53.9 | 43.2 | 35.2 |
| D 5 | 42.2 | 41.3 | 48.5 | 58.7 | 48.0 | 44.8 | 54.0 | 79.1 | 94.2 | 90.8 | 66.2 | 55.6 |
| D 7 | 28.5 | 25.2 | 76.4 | 43.4 | 47.4 | 41.7 | 63.2 | 55.1 | 49.8 | 23.2 | 35.8 | 36.5 |
| D 9 | 26.5 | 33.6 | 32.8 | 29.2 | 26.4 | 25.0 | 27.5 | 62.2 | 53.4 | 19.3 | 32.4 | 40.2 |

Group D - BXQ-350/Oxaliplatin

| ID | 37 | 38 | 39 | 40 | 41 | 42 | 43 | 44 | 45 | 46 | 47 | 48 |
|---|---|---|---|---|---|---|---|---|---|---|---|---|
| BL | 43.0 | 48.0 | 64.8 | 69.7 | 45.4 | 32.0 | 53.2 | 47.9 | 68.3 | 65.2 | 51.4 | 35.7 |
| D 3 | 43.3 | 20.2 | 39.2 | 39.6 | 23.5 | 65.2 | 43.7 | 51.4 | 57.7 | 35.9 | 23.3 | 51.2 |
| D 5 | 35.8 | 46.5 | 73.8 | 53.0 | 79.6 | 81.4 | 38.2 | 47.3 | 79.2 | 70.5 | 34.3 | 83.0 |
| D 7 | 42.3 | 55.5 | 40.4 | 47.8 | 80.9 | 66.8 | 31.3 | 46.5 | 58.4 | 28.6 | 102.0 | 66.9 |
| D 9 | 31.6 | 33.0 | 52.3 | | 80.1 | 43.1 | 26.7 | 37.4 | 45.9 | 24.6 | 39.7 | 49.9 |

Group E - Pregabalin/Oxaliplatin

| ID | 49 | 50 | 51 | 52 | 53 | 54 | 55 | 56 | 57 | 58 | 59 | 60 |
|---|---|---|---|---|---|---|---|---|---|---|---|---|
| BL | 52.9 | 48.6 | 40.2 | 57.5 | 67.5 | 38.5 | 44.8 | 41.3 | 45.6 | 74.9 | 74.7 | 57.3 |
| D 3 | 56.7 | 57.6 | 46.9 | 63.6 | 40.8 | 58.2 | 36.5 | 52.5 | 20.9 | 52.4 | 44.4 | 62.3 |
| D 5 | 22.2 | 41.5 | 39.3 | 61.3 | 40.1 | 30.1 | 42.7 | 45.2 | 68.8 | 43.5 | 91.7 | 73.6 |
| D 7 | 60.2 | 55.5 | 29.3 | 32.1 | 35.2 | 35.8 | 32.5 | 31.4 | 43.2 | 40.3 | 72.5 | 98.2 |
| D 9 | 37.8 | 72.7 | 34.6 | 84.7 | 71.5 | 74.9 | 42.7 | 38.9 | 52.3 | 71.7 | 75.8 | 54.0 |

Table 5: Individual latency to displaying withdrawal behaviour in the cold plate test measured in seconds (s).
The ID numbers indicate the mouse ID.
BL: Baseline; D 3, D 5, D 7, and D 9 refer to Days 3, 5, 7, 9 respectively.

TABLE 6
Summary of results: Thermal sensitivity

| | | | Latency (s) | Latency (s) | Latency (s) | Latency (s) | Latency (s) |
|---|---|---|---|---|---|---|---|
| Group | Treatment | N | Baseline | D 3 | D 5 | D 7 | D 9 |
| A | Vehicle/ | 11 | 58.009 ± | 48.173 ± | 52.118 ± | 46.436 ± | 52.845 ± |
| | Vehicle | | 5.194 | 4.973 | 3.045 | 3.002 | 6.760 |
| B | Oxaliplatin/ | 12 | 54.717 ± | 25.583 ± | 28.567 ± | 32.475 ± | 22.050 ± |
| | Vehicle | | 2.979 | 1.762 | 2.319 | 4.052 | 2.394 |
| C | Vehicle/ | 12 | 52.871 ± | 42.475 ± | 60.283 ± | 43.850 ± | 34.042 ± |
| | BXQ 350 | | 3.560 | 4.726 | 5.331 | 4.558 | 3.579 |
| D | Oxaliplatin/ | 12 | 52.029 ± | 41.183 ± | 60.217 ± | 55.617 ± | 42.209 ± |
| | BXQ 350 | | 3.632 | 4.057 | 5.610 | 6.108 | 4.660 |
| E | Oxaliplatin/ | 12 | 53.629 ± | 49.400 ± | 50.000 ± | 47.183 ± | 59.300 ± |
| | Pregabalin | | 3.737 | 3.555 | 5.740 | 6.049 | 5.138 |

Table 6: Latency to withdrawal behaviour in the cold plate test (15° C.) measured in seconds (s).
Data summary from individual groups and timepoints.
Values are shown as mean ± SEM.

No iatrogenic effects were demonstrated in the oxaliplatin model of chemotherapy-induced neuropathy (CIN). Nevertheless, von-Frey mechanical allodynia was always tested first, followed by cold plate thermal allodynia.

Body Weights: The mice were weighed daily starting 1 day prior to treatment through day 9. Individual body weights are shown in Table 7 below.

TABLE 7
Individual body weights (g)
Group A - Vehicle/Vehicle

| ID | 1 | 2 | 3 | 4 | 5 | 6 | 8 | 9 | 10 | 11 | 12 |
|---|---|---|---|---|---|---|---|---|---|---|---|
| D −1 | 18.8 | 22.0 | 18.6 | 22.3 | 21.0 | 22.4 | 22.2 | 21.2 | 21.6 | 21.9 | 21.7 |
| D 0 | 19.8 | 23.1 | 19.9 | 23.3 | 22.2 | 23.3 | 23.0 | 22.2 | 22.7 | 23.5 | 22.9 |
| D 1 | 20.2 | 23.3 | 20.4 | 23.2 | 22.3 | 23.7 | 23.1 | 22.6 | 23.0 | 23.1 | 22.6 |
| D 2 | 23.0 | 22.7 | 21.1 | 23.5 | 22.2 | 24.0 | 23.5 | 22.9 | 23.4 | 23.6 | 23.0 |
| D 3 | 20.8 | 23.3 | 21.2 | 23.6 | 22.3 | 23.4 | 23.5 | 23.1 | 23.4 | 23.7 | 23.5 |
| D 4 | 21.9 | 23.5 | 21.9 | 23.5 | 22.5 | 24.3 | 23.6 | 23.0 | 23.4 | 23.6 | 23.6 |
| D 5 | 20.9 | 23.0 | 22.0 | 23.5 | 22.3 | 24.7 | 23.3 | 23.7 | 24.0 | 23.0 | 23.4 |
| D 6 | 19.5 | 23.1 | 22.4 | 23.2 | 22.2 | 24.6 | 23.1 | 23.3 | 23.8 | 23.0 | 23.6 |
| D 7 | 21.9 | 23.3 | 23.0 | 23.9 | 22.5 | 25.1 | 23.4 | 23.6 | 24.2 | 23.5 | 24.4 |
| D 8 | 22.0 | 23.2 | 22.6 | 23.3 | 22.3 | 25.3 | 23.1 | 23.6 | 24.6 | 22.9 | 23.7 |
| D 9 | 22.3 | 23.8 | 23.5 | 24.5 | 22.6 | 26.2 | 24.0 | 24.4 | 25.0 | 23.7 | 25.1 |

Group B - Oxaliplatin/Vehicle

| ID | 13 | 14 | 15 | 16 | 17 | 18 | 19 | 20 | 21 | 22 | 23 | 24 |
|---|---|---|---|---|---|---|---|---|---|---|---|---|
| D −1 | 20.5 | 22.0 | 19.8 | 24.3 | 24.5 | 21.9 | 21.3 | 22.6 | 23.3 | 22.1 | 21.5 | 19.9 |
| D 0 | 21.5 | 22.7 | 20.5 | 25.2 | 25.4 | 23.0 | 22.5 | 23.3 | 24.5 | 22.8 | 22.4 | 20.6 |
| D 1 | 21.1 | 22.7 | 19.7 | 24.3 | 24.5 | 22.1 | 21.8 | 21.1 | 23.4 | 22.2 | 24.4 | 20.5 |
| D 2 | 21.5 | 23.7 | 20.5 | 24.6 | 24.9 | 22.8 | 22.0 | 21.9 | 23.4 | 22.3 | 22.5 | 20.9 |
| D 3 | 21.3 | 23.8 | 20.4 | 24.9 | 24.2 | 23.2 | 22.0 | 22.4 | 23.0 | 22.9 | 22.4 | 20.7 |
| D 5 | 20.7 | 23.5 | 20.6 | 25.4 | 23.7 | 23.2 | 22.1 | 22.3 | 23.2 | 23.5 | 22.8 | 21.0 |
| D 6 | 20.9 | 23.6 | 21.3 | 25.6 | 24.4 | 23.0 | 22.3 | 22.8 | 23.8 | 22.8 | 22.4 | 20.7 |
| D 7 | 21.8 | 24.5 | 21.6 | 26.4 | 25.1 | 23.8 | 22.7 | 23.5 | 24.7 | 23.6 | 23.1 | 21.5 |
| D 8 | 22.1 | 24.0 | 20.9 | 25.7 | 24.9 | 23.6 | 22.6 | 23.1 | 24.4 | 23.1 | 23.0 | 21.0 |
| D 9 | 22.4 | 24.8 | 21.6 | 26.4 | 25.3 | 24.1 | 23.5 | 23.3 | 24.5 | 23.6 | 23.1 | 21.2 |

Group C - BXQ-350/Vehicle

| ID | 25 | 26 | 27 | 28 | 29 | 30 | 31 | 32 | 33 | 34 | 35 | 36 |
|---|---|---|---|---|---|---|---|---|---|---|---|---|
| D −1 | 22.8 | 22.8 | 24.2 | 22.7 | 23.6 | 20.5 | 19.8 | 22.7 | 23.8 | 21.9 | 22.4 | 21.4 |
| D 0 | 24.1 | 23.7 | 25.1 | 23.5 | 25.4 | 21.2 | 20.6 | 23.1 | 24.7 | 22.7 | 23.6 | 21.9 |
| D 1 | 24.4 | 23.9 | 25.5 | 23.6 | 25.2 | 21.2 | 20.6 | 23.1 | 24.9 | 22.9 | 23.5 | 22.4 |
| D 2 | 24.6 | 24.0 | 26.0 | 24.0 | 25.7 | 21.8 | 21.3 | 23.9 | 25.3 | 23.4 | 23.8 | 22.5 |
| D 3 | 25.2 | 24.1 | 25.8 | 24.2 | 25.7 | 21.3 | 21.3 | 23.8 | 25.0 | 22.8 | 23.8 | 22.5 |
| D 4 | 25.0 | 23.7 | 25.8 | 24.3 | 25.6 | 21.4 | 21.1 | 24.1 | 25.4 | 23.1 | 24.0 | 22.6 |
| D 5 | 24.6 | 24.5 | 26.3 | 24.7 | 26.1 | 21.5 | 21.4 | 24.1 | 25.2 | 23.5 | 24.2 | 22.8 |
| D 6 | 25.1 | 24.1 | 26.6 | 25.0 | 25.9 | 21.2 | 21.1 | 24.2 | 25.7 | 23.3 | 24.5 | 23.1 |
| D 7 | 25.2 | 24.4 | 27.0 | 25.4 | 26.3 | 21.6 | 21.6 | 24.7 | 25.5 | 23.6 | 25.1 | 23.1 |
| D 8 | 25.1 | 23.8 | 26.2 | 24.8 | 26.3 | 21.0 | 21.0 | 24.1 | 25.1 | 23.3 | 24.4 | 23.0 |
| D 9 | 25.5 | 24.9 | 27.1 | 25.8 | 26.8 | 21.6 | 21.7 | 25.4 | 25.8 | 24.0 | 25.5 | 23.2 |

Group D - BXQ-350/Oxaliplatin

| ID | 37 | 38 | 39 | 40 | 41 | 42 | 43 | 44 | 45 | 46 | 47 | 48 |
|---|---|---|---|---|---|---|---|---|---|---|---|---|
| D −1 | 19.7 | 23.7 | 23.3 | 17.7 | 21.7 | 20.8 | 21.2 | 24.1 | 22.1 | 23.4 | 22.5 | 21.1 |
| D 0 | 20.2 | 24.7 | 24.7 | 18.6 | 22.8 | 22.0 | 22.1 | 25.4 | 23.5 | 24.7 | 24.0 | 22.6 |
| D 1 | 19.6 | 23.6 | 23.3 | 17.4 | 21.9 | 20.8 | 21.2 | 23.9 | 23.0 | 23.4 | 22.3 | 21.3 |
| D 2 | 19.8 | 23.5 | 23.2 | 17.4 | 22.4 | 21.3 | 23.9 | 24.0 | 23.7 | 23.8 | 22.8 | 21.9 |
| D 3 | 20.1 | 22.9 | 23.4 | 17.3 | 22.8 | 21.5 | 20.9 | 23.9 | 24.1 | 23.4 | 22.3 | 21.4 |
| D 4 | 20.0 | 22.8 | 22.8 | 17.7 | 23.0 | 21.6 | 21.1 | 23.4 | 24.1 | 23.7 | 22.7 | 21.8 |
| D 5 | 20.2 | 22.6 | 23.6 | 17.9 | 23.2 | 22.0 | 21.5 | 23.9 | 23.9 | 24.2 | 22.7 | 21.8 |
| D 6 | 20.3 | 24.1 | 23.7 | 18.8 | 23.2 | 22.0 | 21.7 | 24.8 | 24.1 | 24.7 | 23.3 | 22.3 |
| D 7 | 20.8 | 24.9 | 24.8 | 19.2 | 23.7 | 22.4 | 22.0 | 24.9 | 24.5 | 24.7 | 23.6 | 25.4 |
| D 8 | 20.0 | 24.2 | 23.8 | 17.4 | 23.4 | 22.4 | 21.8 | 25.0 | 23.4 | 24.9 | 23.7 | 22.9 |
| D 9 | 20.6 | 25.4 | 24.6 | | 24.6 | 23.3 | 22.5 | 25.8 | 24.7 | 25.6 | 24.7 | 23.7 |

Group E - Pregabalin/Oxaliplatin

| ID | 49 | 50 | 51 | 52 | 53 | 54 | 55 | 56 | 57 | 58 | 59 | 60 |
|---|---|---|---|---|---|---|---|---|---|---|---|---|
| D −1 | | | | | | | | | | | | |
| D 0 | 25.4 | 20.6 | 21.7 | 23.3 | 22.6 | 22.2 | 24.5 | 24.0 | 23.9 | 23.8 | 21.1 | 22.3 |
| D 1 | 23.9 | 19.7 | 20.8 | 22.2 | 21.4 | 21.3 | 23.7 | 22.9 | 22.8 | 22.7 | 20.5 | 21.3 |
| D 2 | 24.8 | 20.0 | 20.6 | 22.3 | 21.7 | 21.3 | 24.0 | 23.3 | 23.1 | 23.4 | 20.8 | 21.6 |
| D 3 | 24.7 | 19.5 | 20.0 | 21.9 | 21.8 | 21.0 | 24.0 | 23.2 | 22.7 | 23.1 | 20.8 | 20.8 |
| D 4 | 24.0 | 18.9 | 19.5 | 21.4 | 20.8 | 20.8 | 23.2 | 22.8 | 22.4 | 22.5 | 20.8 | 20.3 |
| D 5 | 24.8 | 19.0 | 19.2 | 22.0 | 21.5 | 21.2 | 24.2 | 22.2 | 23.2 | 23.5 | 21.6 | 20.3 |
| D 6 | | | | | | | | | | | | |
| D 7 | 26.0 | 20.3 | 20.2 | 23.2 | 23.5 | 22.7 | 25.6 | 24.3 | 24.5 | 24.8 | 22.2 | 21.7 |
| D 8 | 25.3 | 20.0 | 20.8 | 23.1 | 23.2 | 22.6 | 25.1 | 24.5 | 24.1 | 24.0 | 22.2 | 21.6 |
| D 9 | 26.4 | 21.2 | 20.9 | 23.8 | 24.0 | 23.4 | 25.6 | 24.8 | 24.8 | 24.8 | 23.1 | 22.5 |

### BQX-350 and Pregabalin Formulations

Warm vials of BQX-350 were brought to room temperature and reconstituted with 4 mL water for injection via syringe. The vials were gently agitated and/or vortexed for 15-20 sec and allowed to sit at room temp for 5 mins. The rehydrated solution contained 2.2 mg/mL SapC (8.8 mg SapC per vial). About 10 ml of the formulation was injected to achieve a target dose of 20 mg/kg in each mouse. Pregabalin was reconstituted in water and injected to achieve a target dose of 30 mg/kg in each mouse.

### Statistical Analysis

A two-way repeated measures ANOVA with ‘treatment’ as a between subjects effect, and ‘day’ as a within subjects effect was used (using GraphPad Prism version 9.0 for Windows, GraphPad Software, San Diego California USA), followed by Dunnett's multi comparisons test. Data is presented as mean±SEM.

### Results of Study 1

Administration of oxaliplatin induced consistent and persistent mechanical allodynic response and increased thermal (cold) sensitivity as measured from day 3 post oxaliplatin administration to day 9, the last day of behavioral assessment.

Pregabalin administration at a dose of 30 mg/kg, 2 hours before behavioral assessment, reduced mechanical allodynia and reduced thermal sensitivity in oxaliplatin treated animals. Pregabalin was used as a standard positive control for the assays.

BXQ-350 showed a statistically significant protective effect in oxaliplatin treated animals in the cold plate test, but not with mechanical allodynia assessment. Administration of BXQ-350 alone resulted in a trend towards increased mechanical allodynia. This trend was not apparent in the cold hypersensitivity test.

### Study 2

### Animals

60 adult male C57/BL6 mice (Charles River), 6-7 weeks of age, were housed in groups of 5 in Perspex cages, in a controlled environment of constant temperature and humidity (temperature: 21±1° C., light: dark cycle of 12:12 hours), with food and water available ad libitum. Animals were allowed to recover from transportation for at least one week before commencing experiments. The mice were assigned to the various groups shown in Table 8.

TABLE 8
Animal groups

| | | | | | Dose | |
|---|---|---|---|---|---|---|
| Group | CIN treatment | Dose Level | Days of Test | | Level and | No. of |
| ID | (Day 0) | and route | Article Dose | Test Article | route | Animals |
| A | Vehicle | 10 mL/kg IP | −3, −2, −1 and 1 to 9 | Vehicle | 10 mL/kg IP | 12 |
| B | Oxaliplatin | 10 mg/kg IP | −3, −2, −1 and 1 to 9 | Vehicle | 10 mL/kg IP | 12 |
| C | Oxaliplatin | 10 mg/kg IP | −3, −2, −1 and 1 to 9 | BXQ-350 | 2 mg/kg IP | 12 |
| D | Oxaliplatin | 10 mg/kg IP | −3, −2, −1 and 1 to 9 | BXQ-350 | 10 mg/kg IP | 12 |
| E | Oxaliplatin | 10 mg/kg IP | 3, 5, 7, and 9 | Pregabalin | 30 mg/kg IP | 12 |

CIN: chemotherapy-induced neuropathy

### Experimental Design

As shown in Table 8, mice (n=12 per group) in groups A-E were administered 10 mg/kg intraperitoneal oxaliplatin or vehicle at day 0, and administered test article as follows: 10 mg/kg vehicle, 2 mg/kg BXQ-350, or 10 mg/kg BXQ-350 on days −3, −2, −1, and 1 to 9; or 30 mg/kg pregabalin on days 3, 5, 7, and 9. FIG. 1B shows the timeline of administration of the various treatments to mice in groups A-E.

### Apparatus and Evaluation of Study 2

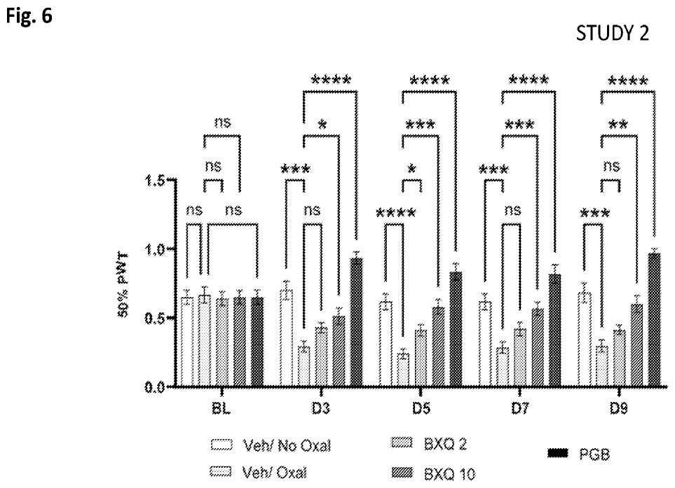

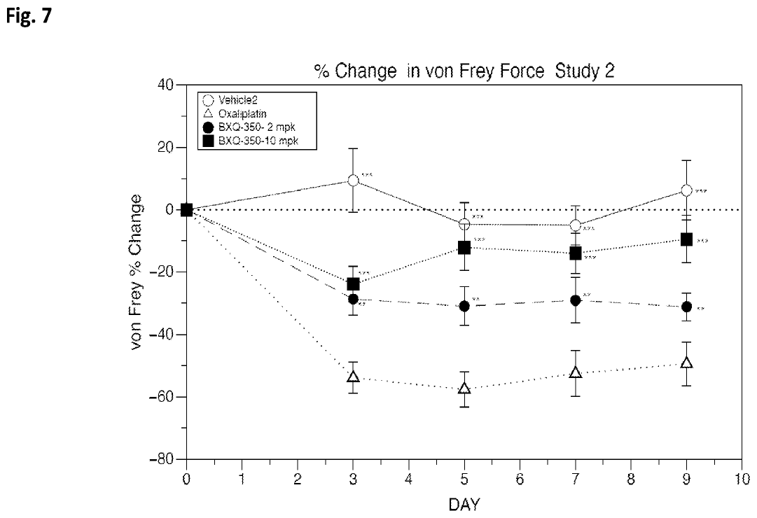

Static mechanical tactile allodynia: The same apparatus and protocol were used as for Study 1. The results of Study 2 are shown in Table 9 (raw data), Table 10 (summary), FIG. 6, and FIG. 7.

TABLE 9
Individual Paw Withdrawal Threshold
50% (von Frey filaments) values (g)
Group A - Vehicle/Vehicle

| ID | 3 | 4 | 7 | 12 | 18 | 25 | 31 | 43 | 45 | 49 | 50 | 54 |
|---|---|---|---|---|---|---|---|---|---|---|---|---|
| D −3 | 1.0 | 0.6 | 0.4 | 1.0 | 0.4 | 0.4 | 1.0 | 0.4 | 1.0 | 0.6 | 0.4 | 0.6 |
| D −2 | 1.0 | 0.4 | 1.0 | 0.6 | 0.6 | 0.4 | 0.6 | 0.6 | 0.6 | 0.6 | 0.6 | 1.0 |
| D −1 | 0.4 | 0.6 | 1.0 | 0.6 | 1.0 | 0.6 | 1.0 | 0.6 | 0.4 | 0.2 | 0.6 | 0.6 |
| D 3 | 1.0 | 0.6 | 1.0 | 0.6 | 1.0 | 0.4 | 0.6 | 1.0 | 0.6 | 0.6 | 0.4 | 0.6 |
| D 5 | 1.0 | 0.6 | 0.6 | 1.0 | 0.6 | 0.6 | 0.6 | 0.4 | 0.6 | 0.4 | 0.4 | 0.6 |
| D 7 | 1.0 | 0.4 | 1.0 | 0.6 | 0.6 | 0.4 | 0.6 | 0.6 | 0.6 | 0.6 | 0.4 | 0.6 |
| D 9 | 1.0 | 0.6 | 0.6 | 1.0 | 1.0 | 0.4 | 0.6 | 0.6 | 1.0 | 0.6 | 0.4 | 0.4 |

Group B - Oxaliplatin/Vehicle

| ID | 5 | 8 | 13 | 14 | 20 | 32 | 33 | 34 | 52 | 53 | 56 | 59 |
|---|---|---|---|---|---|---|---|---|---|---|---|---|
| D −3 | 0.6 | 1.0 | 0.6 | 0.6 | 1.0 | 0.6 | 0.6 | 0.4 | 0.4 | 0.6 | 1.0 | 1.0 |
| D −2 | 0.4 | 1.0 | 0.2 | 0.6 | 1.0 | 0.6 | 0.6 | 0.4 | 0.6 | 0.6 | 1.0 | 0.6 |
| D −1 | 0.6 | 0.4 | 0.6 | 0.6 | 1.0 | 0.6 | 1.0 | 0.6 | 0.6 | 0.4 | 1.0 | 1.0 |
| D 3 | 0.1 | 0.4 | 0.2 | 0.4 | 0.4 | 0.2 | 0.4 | 0.4 | 0.2 | 0.2 | 0.4 | 0.4 |
| D 5 | 0.4 | 0.2 | 0.2 | 0.4 | 0.4 | 0.2 | 0.2 | 0.2 | 0.2 | 0.4 | 0.2 | 0.2 |
| D 7 | 0.4 | 0.4 | 0.4 | 0.4 | 0.2 | 0.1 | 0.2 | 0.4 | 0.1 | 0.2 | 0.4 | 0.4 |
| D 9 | 0.2 | 0.4 | 0.2 | 0.4 | 0.2 | 0.4 | 0.6 | 0.4 | 0.4 | 0.2 | 0.2 | 0.2 |

Group C - Oxaliplatin/BXQ-350 - 2 mg/kg

| ID | 1 | 9 | 16 | 17 | 21 | 22 | 23 | 37 | 39 | 44 | 46 | 57 |
|---|---|---|---|---|---|---|---|---|---|---|---|---|
| D −3 | 0.6 | 0.6 | 0.6 | 0.6 | 0.4 | 0.4 | 0.6 | 0.6 | 0.6 | 0.6 | 0.6 | 0.4 |
| D −2 | 1.0 | 0.6 | 0.6 | 0.4 | 1.0 | 0.6 | 0.6 | 0.6 | 0.6 | 0.6 | 0.4 | 0.4 |
| D −1 | 0.4 | 1.0 | 0.2 | 0.6 | 1.0 | 0.6 | 1.0 | 0.6 | 0.4 | 0.6 | 1.0 | 0.6 |
| D 3 | 0.4 | 0.6 | 0.2 | 0.6 | 0.6 | 0.4 | 0.4 | 0.4 | 0.4 | 0.4 | 0.4 | 0.4 |
| D 5 | 0.4 | 0.6 | 0.4 | 0.6 | 0.6 | 0.4 | 0.2 | 0.4 | 0.4 | 0.4 | 0.4 | 0.2 |
| D 7 | 0.6 | 0.6 | 0.4 | 0.2 | 0.4 | 0.4 | 0.1 | 0.6 | 0.4 | 0.4 | 0.6 | 0.4 |
| D 9 | 0.6 | 0.4 | 0.4 | 0.4 | 0.6 | 0.2 | 0.4 | 0.4 | 0.4 | 0.4 | 0.4 | 0.4 |

Group D - Oxaliplatin/BXQ-350 - 10 mg/kg

| ID | 6 | 10 | 24 | 26 | 28 | 29 | 36 | 38 | 42 | 47 | 55 | 60 |
|---|---|---|---|---|---|---|---|---|---|---|---|---|
| D −3 | 0.6 | 0.6 | 1.0 | 0.6 | 1.0 | 0.6 | 1.0 | 0.6 | 0.6 | 0.6 | 0.6 | 0.6 |
| D −2 | 0.6 | 0.6 | 0.6 | 1.0 | 0.6 | 1.0 | 1.0 | 0.6 | 0.4 | 0.6 | 0.6 | 0.6 |
| D −1 | 0.6 | 1.0 | 0.2 | 0.6 | 0.6 | 1.0 | 0.6 | 0.6 | 0.6 | 0.6 | 0.6 | 0.4 |
| D 3 | 0.4 | 0.6 | 0.6 | 0.6 | 0.6 | 1.0 | 0.6 | 0.4 | 0.4 | 0.2 | 0.4 | 0.4 |
| D 5 | 0.6 | 0.6 | 0.6 | 0.6 | 1.0 | 0.6 | 0.6 | 0.4 | 0.2 | 0.6 | 0.6 | 0.6 |
| D 7 | 0.6 | 0.6 | 0.4 | 0.6 | 0.6 | 0.4 | 1.0 | 0.4 | 0.6 | 0.6 | 0.4 | 0.6 |
| D 9 | 0.6 | 0.6 | 0.4 | 0.6 | 1.0 | 1.0 | 0.4 | 0.4 | 0.6 | 0.6 | 0.4 | 0.6 |

Group E - Oxaliplatin/Pregabalin

| ID | 2 | 11 | 15 | 19 | 27 | 30 | 35 | 40 | 41 | 48 | 51 | 58 |
|---|---|---|---|---|---|---|---|---|---|---|---|---|
| D −3 | 0.6 | 0.4 | 0.6 | 0.6 | 0.6 | 0.6 | 1.0 | 0.4 | 0.6 | 0.6 | 1.0 | 0.6 |
| D −2 | 0.4 | 0.6 | 0.4 | 0.6 | 0.6 | 0.6 | 1.0 | 0.6 | 0.6 | 0.6 | 0.6 | 0.6 |
| D −1 | 1.0 | 1.0 | 0.4 | 0.4 | 1.0 | 0.6 | 1.0 | 0.6 | 0.2 | 0.6 | 1.0 | 0.6 |
| D 3 | 1.0 | 0.6 | 1.0 | 1.0 | 0.6 | 1.0 | 1.0 | 1.0 | 1.0 | 1.0 | 1.0 | 1.0 |
| D 5 | 1.0 | 1.0 | 1.0 | 0.6 | 0.6 | 1.0 | 1.0 | 0.6 | 1.0 | 1.0 | 0.6 | 0.6 |
| D 7 | 1.0 | 0.6 | 1.0 | 0.4 | 1.0 | 1.0 | 0.6 | 0.6 | 1.0 | 1.0 | 1.0 | 0.6 |
| D 9 | 1.0 | 0.6 | 1.0 | 1.0 | 1.0 | 1.0 | 1.0 | 1.0 | 1.0 | 1.0 | 1.0 | 1.0 |

Table 9: Individual Paw Withdrawal Threshold 50% (von Frey filaments) values (g).
The ID numbers indicate the mouse ID.
BL: Baseline; D 3, D 5, D 7, and D 9 refer to Days 3, 5, 7, and 9 respectively.

TABLE 10
Summary of results: Mechanical allodynia

| | | | PWT | PWT | PWT | PWT | PWT | PWT | PWT |
|---|---|---|---|---|---|---|---|---|---|
| | | | 50% (g) | 50% (g) | 50% (g) | 50% (g) | 50% (g) | 50% (g) | 50% (g) |
| Group | Treatment | N | D −3 | D −2 | D −1 | D 3 | D 5 | D 7 | D 9 |
| A | Vehicle/ | 12 | 0.650 ± | 0.667 ± | 0.630 ± | ****0.700 ± | ****0.617 ± | ***0.617 ± | ***0.683 ± |
| | Vehicle | | 0.078 | 0.062 | 0.075 | 0.067 | 0.058 | 0.058 | 0.072 |
| B | Oxaliplatin/ | 12 | 0.700 ± | 0.630 ± | 0.700 ± | 0.293 ± | 0.240 ± | 0.285 ± | 0.297 ± |
| | Vehicle | | .067 | 0.075 | 0.067 | 0.039 | 0.034 | 0.042 | 0.044 |
| C | Oxaliplatin/ | 12 | 0.550 ± | 0.617 ± | 0.663 ± | 0.430 ± | *0.410 ± | 0.419 ± | 413 ± |
| | BXQ 350 2 | | 0.026 | 0.058 | 0.081 | 0.036 | 0.042 | 0.049 | 0.032 |
| D | Oxaliplatin/ | 12 | 0.700 ± | 0.683 ± | 0.613 ± | *0.513 ± | **0.580 ± | ***0.567 ± | **0.600 ± |
| | BXQ 350 10 | | 0.052 | 0.058 | 0.065 | 0.059 | 0.054 | 0.048 | 0.060 |
| E | Oxaliplatin/ | 12 | 0.633 ± | 0.600 ± | 0.697 ± | ****0.933 ± | ****0.833 ± | ****0.817 ± | ****0.967 ± |
| | Pregabalin | | 0.054 | 0.043 | 0.085 | 0.045 | 0.059 | 0.67 | 0.033 |

Table 10: Left hind paw Paw Withdrawal Threshold 50% (g).
Two-way ANOVA showed a main effect of time (F (4.834, 265.9) = 4.829; p < 0.001), a main effect of treatment (F (4, 55) = 17.50; p < 0.0001) and a significant interaction between time and treatment factors (F(24, 330) = 6.502.
*, **, ***, and **** indicate statistically significant difference with p < 0.05, p < 0.01, p < 0.001 and p < 0.0001 respectively when compared to the Oxaliplatin/Vehicle group in the same timepoint.
Values are shown as mean ± SEM.

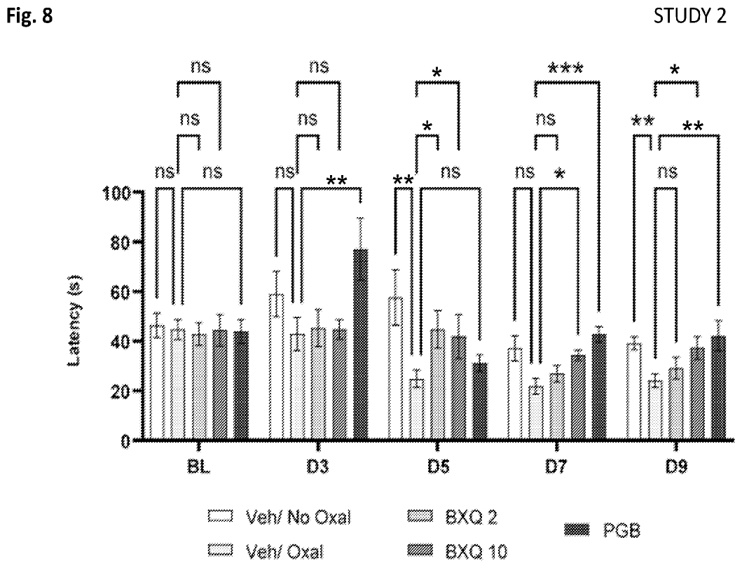

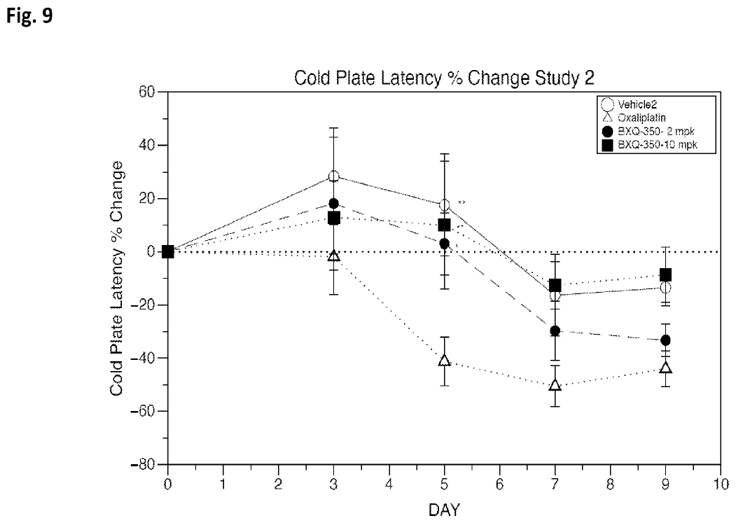

Thermal hypersensitivity: The same apparatus and protocol were used as for Study 1. The results of Study 2 are shown in Table 11 (raw data), Table 12 (summary), FIG. 8, and FIG. 9.

TABLE 11
Individual latency to displaying withdrawal behaviour in the cold plate test (s)
Group A - Vehicle/Vehicle

| ID | 3 | 4 | 7 | 12 | 18 | 25 | 31 | 43 | 45 | 49 | 50 | 54 |
|---|---|---|---|---|---|---|---|---|---|---|---|---|
| D −3 | 36.5 | 60.2 | 43.9 | 44.7 | 53.6 | 33.2 | 53.0 | 51.3 | 65.3 | 58.3 | 35.0 | 42.9 |
| D −2 | 24.3 | 72.1 | 33.5 | 67.3 | 57.7 | 92.9 | 29.3 | 90.1 | 46.2 | 32.7 | 48.6 | 24.1 |
| D −1 | 26.0 | 28.0 | 31.3 | 46.8 | 40.1 | 64.8 | 44.8 | 54.0 | 54.3 | 29.5 | 42.7 | 32.0 |
| D 3 | 38.9 | 45.4 | 67.7 | 34.7 | 33.8 | 69.3 | 24.8 | 91.8 | 136.0 | 54.9 | 36.8 | 73.6 |
| D 5 | 27.4 | 150.0 | 53.1 | 41.3 | 68.5 | 69.8 | 46.8 | 64.1 | 107.3 | 15.5 | 26.1 | 20.5 |
| D 7 | 18.2 | 24.7 | 15.0 | 50.9 | 30.0 | 44.0 | 30.8 | 35.7 | 34.7 | 59.9 | 26.6 | 74.5 |
| D 9 | 29.6 | 45.2 | 30.1 | 44.4 | 50.1 | 37.2 | 26.8 | 33.9 | 49.6 | 50.4 | 30.0 | 41.3 |

Group B - Oxaliplatin/Vehicle

| ID | 5 | 8 | 13 | 14 | 20 | 32 | 33 | 34 | 52 | 53 | 56 | 59 |
|---|---|---|---|---|---|---|---|---|---|---|---|---|
| D −3 | 29.9 | 24.7 | 30.0 | 72.0 | 35.3 | 50.6 | 67.4 | 55.6 | 39.6 | 35.1 | 40.1 | 49.9 |
| D −2 | 36.8 | 31.9 | 42.2 | 61.4 | 63.3 | 55.6 | 41.5 | 29.0 | 36.7 | 63.0 | 47.4 | 35.8 |
| D −1 | 26.1 | 24.4 | 53.7 | 64.3 | 91.7 | 31.2 | 45.0 | 37.0 | 33.4 | 32.9 | 45.8 | 42.4 |
| D 3 | 18.8 | 19.9 | 34.2 | 36.8 | 23.9 | 77.7 | 54.5 | 28.5 | 39.6 | 69.3 | 84.0 | 28.0 |
| D 5 | 29.8 | 13.1 | 25.5 | 33.8 | 17.4 | 47.1 | 16.4 | 11.8 | 43.8 | 12.7 | 28.7 | 18.6 |
| D 7 | 13.7 | 4.8 | 23.9 | 26.1 | 29.6 | 23.2 | 26.3 | 12.7 | 44.2 | 22.3 | 7.4 | 28.4 |
| D 9 | 25.2 | 18.4 | 23.0 | 35.0 | 23.8 | 12.7 | 30.6 | 6.4 | 33.2 | 23.9 | 39.0 | 17.2 |

Table 11: Individual latency to displaying withdrawal behaviour in the cold plate test measured in seconds (s).
The ID numbers indicate the mouse ID.
BL: Baseline; D 3, D5, D7, and D9 refer to Days 3, 5, 7, 9 respectively.

TABLE 12
Summary of results: Thermal sensitivity

| | | | Latency | Latency | Latency | Latency | Latency | Latency | Latency |
|---|---|---|---|---|---|---|---|---|---|
| | | | (s) | (s) | (s) | (s) | (s) | (s) | (s) |
| Group | Treatment | N | D −3 | D −2 | D −1 | D3 | D5 | D7 | D9 |
| A | Vehicle/ | 12 | 48.158 + | 51.567 + | 41.192 + | 58.975 + | ***57.533 + | 37.083 + | 39.050 ? |
| | Vehicle | | 3.00* | 7.078 | 3.552 | 9.084 | 11.273 | 5.048 | 2.560 |
| B | Oxaliplatin/ | 12 | 44.183 + | 45.383 + | 43.992 + | 42.933 + | 24.888 ? | 21.883 + | 24.033 + |
| | Vehicle | | 4.353 | 3.602 | 5.464 | 6.603 | 3.478 | 3.134 | 2.741 |
| C | Oxaliplatin/ | 12 | 43.650 + | 41.050 + | 44.550 + | 45.283 + | 44.6920 | 26.900 + | 29.183 + |
| | BXQ 350 2 | | 4.479 | 4.660 | 5.615 | 7.379 | + 7.506 | 3.391 | 4.341 |
| D | Oxaliplatin/ | 12 | 40.458 + | 45.883 + | 42.967 + | 44.750 + | 41.908 + | 34.367 + | 37.300 + |
| | BXQ 350 10 | | 4.727 | 5.714 | 7.800 | 3.956 | 8.909 | 2.002 | 4.668 |
| E | Oxaliplatin/ | 12 | 43.908 + | 44.725 + | 43.175 + | ***77.083 + | 31.188 + | *42.792 + | 42.063 + |
| | Pregabalin | | 4.255 | 6.470 | 5.222 | 12.587 | 3.368 | 3.036 | 6.132 |

Table 12: Latency to withdrawal behaviour in the cold plate test (15° C.).
Two-way ANOVA showed a main effect of time (F (6, 330) = 8.849; p < 0.0001), a main effect of treatment (F (4, 55) = 2.775; p = 0.0358) and a significant interaction between time and treatment factors (F (24, 330) = 8.849.
*, **, and ***, indicate statistically significant difference with p < 0.05, p < 0.01, and p < 0.001 respectively when compared to the Oxaliplatin/Vehicle Group in the same timepoint.
Values are shown as mean ± SEM.

Body Weights: The mice were weighed daily starting 1 day prior to treatment through day 9. Individual body weights are shown in Table 13 below.

TABLE 13
Individual body weights (g)
Group A - Vehicle/Vehicle

| ID | 3 | 4 | 7 | 12 | 18 | 25 | 31 | 43 | 45 | 49 | 50 | 54 |
|---|---|---|---|---|---|---|---|---|---|---|---|---|
| D −3 | 26.0 | 23.0 | 23.0 | 26.0 | 22.0 | 19.0 | 24.0 | 25.0 | 25.0 | 21.0 | 23.0 | 24.0 |
| D −2 | 25.2 | 23.3 | 22.5 | 25.6 | 21.4 | 18.8 | 24.5 | 25.2 | 24.8 | 21.3 | 22.9 | 24.4 |
| D −1 | 25.2 | 23.4 | 22.4 | 25.7 | 21.9 | 18.8 | 24.5 | 25.6 | 25.0 | 20.9 | 22.4 | 24.0 |
| D 0 | 25.8 | 23.5 | 22.7 | 26.4 | 22.2 | 19.2 | 24.9 | 25.8 | 26.5 | 20.9 | 23.1 | 24.2 |
| D 1 | 25.9 | 23.8 | 22.8 | 26.7 | 22.4 | 19.3 | 25.2 | 26.6 | 25.6 | 21.2 | 23.5 | 24.7 |
| D 2 | 26.3 | 23.9 | 23.0 | 26.7 | 22.6 | 19.5 | 25.0 | 26.5 | 25.7 | 21.3 | 23.5 | 24.7 |
| D 3 | 21.7 | 23.3 | 21.6 | 25.5 | 21.1 | 18.7 | 24.6 | 25.3 | 25.1 | 20.6 | 20.1 | 24.2 |
| D 4 | 25.6 | 24.7 | 22.5 | 26.5 | 22.2 | 19.6 | 24.8 | 26.5 | 25.9 | 21.0 | 21.4 | 24.6 |
| D 5 | 25.7 | 24.5 | 22.5 | 26.9 | 22.9 | 19.6 | 23.8 | 26.3 | 26.2 | 21.6 | 21.6 | 24.6 |
| D 6 | 25.6 | 24.0 | 22.6 | 26.7 | 22.1 | 19.7 | 24.6 | 26.8 | 25.6 | 21.6 | 20.9 | 24.7 |
| D 7 | 25.9 | 24.4 | 22.5 | 27.5 | 22.5 | 19.5 | 25.0 | 26.7 | 26.1 | 21.2 | 21.6 | 24.7 |
| D 8 | 25.1 | 24.0 | 22.1 | 26.4 | 22.6 | 19.5 | 24.4 | 26.3 | 25.7 | 21.2 | 21.6 | 24.3 |
| D 9 | 25.9 | 24.7 | 22.7 | 26.9 | 23.0 | 19.5 | 24.6 | 25.8 | 25.9 | 21.2 | 21.8 | 24.7 |

Group B - Oxaliplatin/Vehicle

| ID | 5 | 8 | 13 | 14 | 20 | 32 | 33 | 34 | 52 | 53 | 56 | 59 |
|---|---|---|---|---|---|---|---|---|---|---|---|---|
| D −3 | 26.0 | 26.0 | 24.0 | 24.0 | 23.0 | 24.0 | 25.0 | 22.0 | 25.0 | 22.0 | 24.0 | 23.0 |
| D −2 | 25.4 | 25.4 | 23.9 | 23.0 | 23.5 | 23.8 | 24.1 | 21.8 | 25.2 | 21.9 | 23.8 | 23.3 |
| D −1 | 25.3 | 25.5 | 23.8 | 22.9 | 22.9 | 24.1 | 24.3 | 21.6 | 26.1 | 21.8 | 23.6 | 22.9 |
| D 0 | 26.0 | 26.0 | 24.5 | 23.6 | 23.2 | 24.6 | 24.5 | 21.9 | 26.1 | 22.4 | 23.6 | 23.1 |
| D 1 | 24.9 | 24.9 | 23.9 | 23.7 | 22.9 | 23.0 | 23.5 | 20.8 | 25.4. | 21.1 | 23.5 | 22.5 |
| D 2 | 24.8 | 24.6 | 23.8 | 23.8 | 23.2 | 23.0 | 23.6 | 21.0 | 25.0 | 21.3 | 23.2 | 22.3 |
| D 3 | 24.1 | 23.7 | 23.0 | 22.4 | 22.1 | 22.2 | 23.0 | 20.6 | 24.4 | 22.4 | 22.7 | 22.2 |
| D 4 | 25.0 | 25.0 | 23.7 | 23.5 | 23.1 | 22.7 | 23.5 | 21.3 | 24.8 | 23.5 | 23.1 | 22.6 |
| D 5 | 25.2 | 25.0 | 23.8 | 23.5 | 23.3 | 22.4 | 23.0 | 20.6 | 24.9 | 23.8 | 22.8 | 22.8 |
| D 6 | 25.3 | 25.3 | 23.9 | 23.1 | 22.9 | 23.7 | 24.0 | 21.8 | 25.4 | 23.7 | 23.2 | 23.0 |
| D 7 | 25.5 | 25.7 | 24.5 | 23.4 | 23.3 | 23.8 | 24.5 | 21.5 | 25.8 | 23.6 | 23.4 | 22.7 |
| D 8 | 25.1 | 25.3 | 23.6 | 23.0 | 22.7 | 23.4 | 24.1 | 21.2 | 25.4 | 23.3 | 23.4 | 21.5 |
| D 9 | 25.3 | 25.5 | 24.0 | 23.7 | 23.3 | 23.1 | 24.5 | 22.0 | 25.7 | 23.5 | 23.4 | 22.2 |

Group C - Oxaliplatin/BXQ-350 - 2 mg/kg

| ID | 1 | 9 | 16 | 17 | 21 | 22 | 23 | 37 | 39 | 44 | 46 | 57 |
|---|---|---|---|---|---|---|---|---|---|---|---|---|
| D −3 | 25.0 | 24.0 | 25.0 | 26.0 | 23.0 | 25.0 | 25.0 | 23.0 | 26.0 | 22.0 | 23.0 | 24.0 |
| D −2 | 24.8 | 24.0 | 24.1 | 24.9 | 25.7 | 24.9 | 24.9 | 22.9 | 24.8 | 21.5 | 22.7 | 24.2 |
| D −1 | 24.8 | 23.9 | 24.1 | 25.4 | 23.6 | 25.8 | 25.1 | 22.9 | 24.7 | 21.7 | 23.1 | 24.4 |
| D 0 | 25.4 | 24.8 | 24.9 | 25.9 | 23.9 | 26.0 | 25.1 | 24.0 | 25.3 | 22.0 | 23.6 | 24.8 |
| D 1 | 24.7 | 23.5 | 23.5 | 25.4 | 22.6 | 25.0 | 24.5 | 22.7 | 24.4 | 21.2 | 22.6 | 23.7 |
| D 2 | 24.9 | 23.0 | 23.4 | 25.4 | 22.6 | 25.3 | 24.6 | 22.9 | 24.3 | 21.3 | 22.3 | 23.7 |
| D 3 | 24.2 | 22.4 | 22.5 | 24.2 | 21.3 | 25.0 | 23.3 | 22.5 | 23.6 | 20.3 | 21.9 | 23.1 |
| D 4 | 25.1 | 23.3 | 23.5 | 25.3 | 22.6 | 26.0 | 24.6 | 22.6 | 24.3 | 21.4 | 22.2 | 24.0 |
| D 5 | 21.1 | 22.1 | 23.9 | 23.7 | 22.7 | 26.0 | 24.4 | 21.6 | 23.6 | 21.4 | 22.4 | 23.9 |
| D 6 | 25.5 | 23.6 | 24.7 | 26.0 | 22.8 | 26.3 | 25.0 | 22.6 | 24.9 | 21.5 | 22.8 | 24.5 |
| D 7 | 26.1 | 24.6 | 24.9 | 26.4 | 23.1 | 27.4 | 25.1 | 23.1 | 25.1 | 22.0 | 22.7 | 24.9 |
| D 8 | 25.6 | 24.0 | 24.7 | 25.7 | 23.1 | 26.6 | 24.8 | 23.4 | 24.8 | 21.4 | 22.6 | 24.9 |
| D 9 | 26.5 | 24.7 | 25.0 | 26.1 | 23.4 | 27.2 | 25.7 | 23.9 | 24.4 | 21.8 | 23.1 | 24.7 |

Group D - Oxaliplatin/BXQ-350 - 10 mg/kg

| ID | 6 | 10 | 24 | 26 | 28 | 29 | 36 | 38 | 42 | 47 | 55 | 60 |
|---|---|---|---|---|---|---|---|---|---|---|---|---|
| D −3 | 22.0 | 26.0 | 23.0 | 25.0 | 24.0 | 24.0 | 27.0 | 22.0 | 25.0 | 24.0 | 23.0 | 22.0 |
| D −2 | 22.1 | 25.6 | 23.1 | 24.6 | 23.6 | 24.1 | 26.3 | 21.6 | 24.8 | 23.1 | 23.4 | 22.4 |
| D −1 | 22.1 | 25.3 | 23.1 | 24.5 | 23.3 | 24.4 | 26.9 | 21.7 | 24.7 | 23.4 | 23.4 | 22.4 |
| D 0 | 22.3 | 26.0 | 23.5 | 24.6 | 23.4 | 25.1 | 27.1 | 22.0 | 24.5 | 23.1 | 23.4 | 22.9 |
| D 1 | 21.8 | 25.4 | 22.6 | 23.5 | 23.4 | 24.0 | 26.3 | 22.1 | 23.8 | 22.4 | 21.8 | 22.1 |
| D 2 | 21.3 | 25.0 | 22.1 | 23.5 | 23.5 | 24.2 | 26.6 | 22.1 | 23.9 | 22.4 | 21.9 | 21.8 |
| D 3 | 21.1 | 24.1 | 21.1 | 22.2 | 22.4 | 23.9 | 25.5 | 21.4 | 23.1 | 21.5 | 21.3 | 21.4 |
| D 4 | 21.6 | 25.1 | 22.0 | 23.4 | 23.5 | 24.1 | 26.1 | 22.6 | 23.7 | 22.3 | 21.8 | 21.9 |
| D 5 | 21.6 | 24.6 | 21.3 | 22.7 | 22.7 | 22.8 | 25.1 | 21.5 | 23.7 | 22.1 | 22.2 | 21.6 |
| D 6 | 21.8 | 24.7 | 22.1 | 23.8 | 23.8 | 23.9 | 26.6 | 22.6 | 24.9 | 22.4 | 22.4 | 21.6 |
| D 7 | 23.0 | 25.6 | 23.0 | 24.0 | 23.8 | 24.5 | 26.9 | 23.0 | 25.1 | 22.8 | 22.5 | 21.9 |
| D 8 | 22.3 | 25.3 | 23.0 | 24.2 | 23.7 | 24.2 | 26.8 | 22.9 | 24.3 | 22.7 | 22.5 | 22.0 |
| D 9 | 22.6 | 25.4 | 23.3 | 24.7 | 24.1 | 24.3 | 27.1 | 22.5 | 24.2 | 23.0 | 22.4 | 22.1 |

Group E - Oxaliplatin/Pregabalin

| ID | 2 | 11 | 15 | 19 | 27 | 30 | 35 | 40 | 41 | 48 | 51 | 58 |
|---|---|---|---|---|---|---|---|---|---|---|---|---|
| D −3 | 22.0 | 26.0 | 26.0 | 23.0 | 25.0 | 24.0 | 26.0 | 23.0 | 25.0 | 23.0 | 28.0 | 23.0 |
| D −2 | 21.9 | 25.6 | 25.9 | 23.3 | 24.6 | 23.8 | 25.4 | 22.8 | 24.4 | 23.1 | 27.6 | 22.7 |
| D −1 | 21.9 | 23.4 | 25.3 | 23.3 | 24.5 | 24.2 | 25.7 | 23.2 | 24.8 | 23.7 | 27.9 | 23.2 |
| D 0 | 21.9 | 25.7 | 25.7 | 23.6 | 24.9 | 21.8 | 26.2 | 23.4 | 25.1 | 23.6 | 28.1 | 23.7 |
| D 1 | 21.6 | 25.2 | 25.1 | 23.1 | 24.5 | 23.6 | 24.8 | 22.6 | 24.4 | 23.0 | 26.8 | 22.7 |
| D 2 | 21.8 | 25.1 | 25.4 | 23.8 | 23.6 | 23.4 | 25.0 | 22.8 | 24.3 | 23.1 | 27.0 | 22.7 |
| D 3 | 21.8 | 24.7 | 25.5 | 23.4 | 22.4 | 22.9 | 24.8 | 22.8 | 24.0 | 23.0 | 26.6 | 22.6 |
| D 4 | 21.8 | 25.0 | 24.8 | 23.4 | 23.5 | 23.7 | 24.9 | 23.1 | 24.0 | 23.0 | 27.0 | 22.3 |
| D 5 | 21.4 | 25.1 | 26.0 | 23.8 | 24.5 | 23.8 | 25.1 | 23.0 | 24.5 | 24.0 | 27.6 | 22.9 |
| D 6 | 21.8 | 25.5 | 25.5 | 23.5 | 24.3 | 23.9 | 25.0 | 22.8 | 25.1 | 23.0 | 27.1 | 23.0 |
| D 7 | 22.0 | 25.9 | 25.9 | 24.0 | 24.9 | 24.6 | 25.5 | 23.6 | 25.2 | 23.9 | 28.0 | 23.8 |
| D 8 | 22.2 | 25.6 | 25.4 | 23.8 | 24.6 | 24.1 | 24.8 | 23.1 | 24.5 | 23.3 | 27.2 | 23.2 |
| D 9 | 22.1 | 25.6 | 25.8 | 24.3 | 25.0 | 24.8 | 25.1 | 23.5 | 24.9 | 24.0 | 27.8 | 23.7 |

### Results of Study 2

Administration of oxaliplatin induced consistent and persistent mechanical allodynic response on the days assessed. Treatment with BXQ-350 significantly prevented a reduction in PWT (paw withdrawal threshold levels) in a dose dependent manner. On all days assessed, the group treated with BXQ-350 showed significantly higher PWT levels when compared to the Veh/Oxal group while on days 3 and 9, groups treated with BXQ-350 at the dose of 2 mg/kg were not significantly different from the Veh/Oxal Group.

BXQ-350 showed a consistent trend in terms of a protective effect in oxaliplatin treated animals in the cold plate test. On days 7 and 10, the intra-group variation was lower, and some statistically significant differences emerged. On day 7, the BXQ-350 10 mg/kg treated group differed significantly from Veh/Oxal group.

### Conclusion

These results show that BXQ-350 can protect mice from oxaliplatin-induced neuropathic pain in a mouse model of mechanical allodynia as well as oxaliplatin-induced cold hypersensitivity.

### Example 3. Efficacy and Safety of BXQ-350 (SapC-DOPS) to Decrease Oxaliplatin-Induced Sensory Neurotoxicity and to Treat Newly Diagnosed Metastatic Colorectal Carcinoma

A placebo controlled, double blind Phase 2 efficacy and safety to determine (a) the efficacy of BXQ-350 plus mFOLFOX7 (modified FOLFOX7: 5-fluorouracil (5-FU)/leucovorin (LV)/oxaliplatin) and bevacizumab (Avastin) in treating patients with metastatic colorectal carcinoma (mCRC); and (b) whether BXQ-350 decreases the development, intensity and/or duration of oxaliplatin-induced sensory neurotoxicity in subjects with mCRC who are receiving mFOLFOX7 may be structured as follows. mFOLFOX7, a chemotherapeutic regimen in which oxaliplatin (85 mg/meter[m]2 infused with leucovorin calcium (200 mg/m²), is administered to a patient over 2 hours on Day 1, followed by a 46-hour infusion (2400 grams [g]/m²) of 5-fluorouracil (5FU) on Days 1 and 2, every 2 weeks for 6 months, for a total of 12 infusions. Eligible subjects are randomized in a 1:1 ratio to receive either BXQ-350 at 2.4 milligrams (mg)/kilogram (kg) or placebo intravenously administered concurrently with mFOLFOX7 (Cycle 1 Days 1-5, 8, 10, 12, 15, and 22; Cycle 2-6 every 14 days starting on Day 29). The eligible subjects are aged ≥18 years, have newly diagnosed metastatic adenocarcinoma of the colon/rectum, are receiving mFOLFOX7, have an Eastern Cooperative Oncology Group (ECOG) Performance Status of 0 or 1, and have a life expectancy of over 3 months.

Subjects receive either BXQ-350 or placebo concurrently with mFOLFOX7 and bevacizumab therapy per the dosing schedule in Table 14:

TABLE 14
BXQ-350/placebo is administered by IV infusion over 60
minutes ± 5 mins according to the following schedule:

| | | | | | Extended |
|---|---|---|---|---|---|
| | | | | | Treatment |
| | | | | | Period |
| Primary Treatment Period | | | | | Cycle 7-26 |
| Cycle 1 | | | | Cycles 2-6 | (Days 183-720) |
| Week 1 | Week 2 | Week 3 | Week 4 | Weeks 5-24 | Week 25-104 |
| BXQ-350 | BXQ-350 | BXQ-350 | BXQ-350 | BXQ-350 | BXQ-350 |
| OR | OR | OR | OR | OR | or |
| Placebo | Placebo | Placebo | Placebo | Placebo | Placebo |
| Days 1-5 | 3x/week | Once | Once | Once every | Once every |
| (5 consecutive | (every other | | | 14 days | 28 days ± |
| days) | day) | | | (±3 days) | 3 days |
| mFOLFOX7 | | mFOLFOX7 | | mFOLFOX7, | Maintenance |
| Bevacizumab * | | Bevacizumab * | | Bevacizumab * | therapy per |
| Once | | Once | | Every 14 days | attending |
| | | | | (±3 days) | physician |
| | | | | | discretion |

* 2-day mFOLFOX7 regimen: oxaliplatin (85 mg/meter[m]² infused with leucovorin calcium (200 mg/m²) over 2 hours on Day 1, followed by a 46-hour infusion (2400 milligrams [mg]/m²) of 5-FU, every 2 weeks for 6 months (12 infusions total). mFOLFOX7 = infused per institutional policies and procedures. Bevacizumab = 5 mg/kg IV. Infused per institutional policies and procedures.

Study duration: Treatment with BXQ-350 or placebo in combination with mFOLFOX7 and bevacizumab may commence following trial entry for 6 cycles (Primary Treatment Period). Beyond 6 cycles (Extended Treatment Period), all participants without progression or other stopping criteria may continue with maintenance mFOLFOX7 and/or bevacizumab per attending physician discretion. BXQ-350/placebo treatment will transition to a once every 28-day dosing schedule and continue beyond 6 cycles until disease progression or other stopping rules are met for a maximum of 720 days from their Study Day 1 (approximately 26 cycles) with an End of Treatment visit occurring 2 weeks after discontinuation of BXQ-350 therapy. Participants may be followed every 28 days for neuropathy assessment until 548 days from their Study Day 1, and for survival every 12 weeks until day 1,825 from their Study Day 1, death, withdrawal of consent, or end of trial, whichever occurs first.

### Study Objectives:

Treatment of mCRC

One objective may include assessment of the efficacy of BXQ-350 plus modified FOLFOX7 (mFOLFOX7) treatment and bevacizumab in patients with mCRC as measured by the objective response rate (ORR). ORR is determined according to Response Evaluation Criteria in Solid Tumors (RECIST) v1.1 by attending physician assessment.

The secondary objectives may include assessing the efficacy of BXQ-350 plus modified FOLFOX7 (mFOLFOX7) treatment and bevacizumab in patients with mCRC as measured by
(a) overall survival (OS)—defined as the time from the date of randomization until date of death due to any cause;
(b) Progression free survival (PFS)—defined as the time from the date of randomization to the date of disease progression or death;
(c) Duration of Response (DoR)—defined as time from documentation of disease response to disease progression; and
(d) Disease control rate (DCR)—defined as the proportion of participants whose best overall response (BOR) was complete response (CR), partial response (PR) and stable disease (SD) according to RECIST v1.1.

In addition, the pharmacokinetics (PK) of BXQ-350 in combination with mFOLFOX7 and bevacizumab may be characterized. Further, the overall safety and tolerability of BXQ-350 in combination with mFOLFOX7 and bevacizumab vs mFOLFOX7 and bevacizumab alone may be assessed in every participant. For this objective, adverse events are graded by Common Terminology Criteria for Adverse Events [CTCAE]v5). In addition, clinical chemistry, hematology, coagulation and urinalysis, vital signs, physical examination, and weight are determined. Further, a 12-lead electrocardiogram (ECG) is conducted and the Eastern Cooperative Oncology Group (ECOG) Performance Status is determined.

### Treatment of Neuropathy

One objective may include determining whether BXQ-350 decreases the development, intensity, and/or duration of chronic oxaliplatin-induced sensory neurotoxicity in subjects receiving modified FOLFOX7 (mFOLFOX7). The objective is measured utilizing the European Organization for Research and Treatment of Cancer (EORTC) Quality of Life (QLQ) questionnaires (QLC-C30 and CIPN 20).

Other objectives may include
(a) to assess whether BXQ-350 administered with mFOLFOX7 results in greater exposure to oxaliplatin, in terms of number of cycles received and total dose administered;
(b) to assess the overall safety and tolerability of BXQ-350 in subjects diagnosed with mCRC that are receiving treatment with mFOLFOX7, in terms of adverse events; and
(c) to assess whether BXQ-350 will decrease oxaliplatin-associated acute pain syndrome symptoms in subjects receiving mFOLFOX7.

The secondary objectives may be measured, for example, utilizing the CIPN Assessment Tool (Tofthagen C S, et al. (2011) Cancer Nurs. 2011 July-August; 34(4):E10-20) and Post-Oxaliplatin Symptom Questionnaire (Pachman D R, et al (2016) Supportive Care in Cancer. 24(12):5059-68).

Efficacy Assessments for Neuropathy: One efficacy outcome of this pilot study may be the serially measured total sensory neuropathy scores obtained from the 6 individual EORTC QLQ-CIPN20 questions that quantify numbness, tingling, and pain in the fingers (or hands) and toes (or feet); the total sensory neuropathy scores will be transformed to a scale of 0-100 (higher numbers are better). In an example, participants receive questionnaires according to Table 15 below:

TABLE 15
Schedule of Questionnaires

| | | | End of | Monthly Follow Up |
|---|---|---|---|---|
| | Screening | Treatment Cycles | Treatment | (12 months) |
| Symptom | X | Post BXQ-350/Placebo Dosing | X | |
| Experience | | (as applicable) | | |
| Questionnaire | | Weekly during Cycle 1 | | |
| | | Every 2 weeks Cycle 2-6 | | |
| EORTC QLQ | X | Post BXQ-350/Placebo Dosing | X | Monthly |
| Questionnaires | | (as applicable) | | |
| (QLQ-30 and | | Weekly during Cycle 1 | | |
| CIPN 20) | | Every 2 weeks Cycle 1-6 | | |
| CIPN Assessment | X | Post BXQ-350/Placebo Dosing | | Monthly |
| Tool | | (as applicable) | | |
| Questionnaire | | Every 2 weeks Cycle 1-6 | | |
| Post-Oxaliplatin | X | Daily on the 6 days after each | | |
| Questionnaire | | oxaliplatin dosing | | |
| Follow-up Call | | Call the next business day after each | | Monthly |
| | | oxaliplatin dosing to reinforce | | |
| | | daily post-oxaliplatin questionnaire | | |
| | | completion and to answer any questions | | |

The questionnaires used in the endpoint objectives may include the EORTC Questionnaire (Table 16), the CIPN Assessment Tool Questionnaire (Table 17), and the Post-Oxaliplatin Symptom Questionnaire (Table 18) as shown below. The Table 17 Questionnaire was developed and being used with permission by Dr. Cindy Tofthagen (Cancer Nursing, Vol. 00, No. 0, 2011). Table 18 was developed and being used with permission by Dr. Charles Loprinzi.

TABLE 16
EORTC QLQ - CIPN20 Questionnaire

| | Not | A | Quite | Very |
|---|---|---|---|---|
| During the past week: | at All | Little | a Bit | Much |
| Did you have tingling fingers or hands? | 1 | 2 | 3 | 4 |
| Did you have tingling toes or feet? | 1 | 2 | 3 | 4 |
| Did you have numbness in your fingers or hands? | 1 | 2 | 3 | 4 |
| Did you have numbness in your toes or feet? | 1 | 2 | 3 | 4 |
| Did you have shooting or burning pain in your fingers or hands? | 1 | 2 | 3 | 4 |
| Did you have shooting or burning pain in your toes or feet? | 1 | 2 | 3 | 4 |
| Did you have cramps in your hands? | 1 | 2 | 3 | 4 |
| Did you have cramps in your feet? | 1 | 2 | 3 | 4 |
| Did you have problems standing or walking because | 1 | 2 | 3 | 4 |
| of difficulty feeling the ground under your feet? | | | | |
| Did you have difficulty distinguishing | 1 | 2 | 3 | 4 |
| between hot and cold water? | | | | |
| Did you have a problem holding a pen, which | 1 | 2 | 3 | 4 |
| made writing difficult? | | | | |
| Did you have difficulty manipulating small objects with | 1 | 2 | 3 | 4 |
| your fingers (for example, fastening small buttons)? | | | | |
| Did you have difficulty opening a jar or | 1 | 2 | 3 | 4 |
| bottle because of weakness in your hands? | | | | |
| Did you have difficulty walking because your | 1 | 2 | 3 | 4 |
| feet dropped downwards? | | | | |
| Did you have difficulty climbing stairs or getting up | 1 | 2 | 3 | 4 |
| out of a chair because of weakness in your legs? | | | | |
| Were you dizzy when standing up from a | 1 | 2 | 3 | 4 |
| sitting or lying position? | | | | |
| Did you have blurred vision? | 1 | 2 | 3 | 4 |
| Did you have difficulty hearing? | 1 | 2 | 3 | 4 |

Please answer the following question only if you drive a car

| | | | | |
|---|---|---|---|---|
| Did you have difficulty using the pedals? | 1 | 2 | 3 | 4 |

Please answer the following question only if you are a man

| | | | | |
|---|---|---|---|---|
| Did you have difficulty getting or maintaining an erection? | 1 | 2 | 3 | 4 |
| © QLQ-CIPN20 Copyright 2003 EORTC Quality of life | | | | |
| Group. All rights reserved | | | | |

TABLE 17
CIPN Assessment Tool Questionnaire (Quantitative Items)
If you have had any neuropathy symptoms (like numbness, tingling, or pain in your
hands and/or feet) over the last week, how much have these symptoms interfered with:

| | Not at all interfering | | | | | | Completely Interfering | | | | |
|---|---|---|---|---|---|---|---|---|---|---|---|
| Dressing (buttoning, zipping, etc) | 0 | 1 | 2 | 3 | 4 | 5 | 6 | 7 | 8 | 9 | 10 |
| Walking | 0 | 1 | 2 | 3 | 4 | 5 | 6 | 7 | 8 | 9 | 10 |
| Picking up objects | 0 | 1 | 2 | 3 | 4 | 5 | 6 | 7 | 8 | 9 | 10 |
| Holding onto objects | 0 | 1 | 2 | 3 | 4 | 5 | 6 | 7 | 8 | 9 | 10 |
| Driving | 0 | 1 | 2 | 3 | 4 | 5 | 6 | 7 | 8 | 9 | 10 |
| Working | 0 | 1 | 2 | 3 | 4 | 5 | 6 | 7 | 8 | 9 | 10 |
| Participating in hobbies or leisure activities | 0 | 1 | 2 | 3 | 4 | 5 | 6 | 7 | 8 | 9 | 10 |
| Exercising | 0 | 1 | 2 | 3 | 4 | 5 | 6 | 7 | 8 | 9 | 10 |
| Sleeping | 0 | 1 | 2 | 3 | 4 | 5 | 6 | 7 | 8 | 9 | 10 |
| Sexual activity | 0 | 1 | 2 | 3 | 4 | 5 | 6 | 7 | 8 | 9 | 10 |
| Relationships with other people | 0 | 1 | 2 | 3 | 4 | 5 | 6 | 7 | 8 | 9 | 10 |
| Writing | 0 | 1 | 2 | 3 | 4 | 5 | 6 | 7 | 8 | 9 | 10 |
| Usual household chores | 0 | 1 | 2 | 3 | 4 | 5 | 6 | 7 | 8 | 9 | 10 |
| Enjoyment of life | 0 | 1 | 2 | 3 | 4 | 5 | 6 | 7 | 8 | 9 | 10 |

TABLE 18
Post-Oxaliplatin Symptom Questionnaire
Please circle the one number for each item below that best describes you.
1. Did you experience sensitivity to touching cold items
within the last 24 hours?

| | | | | | | | | | | |
|---|---|---|---|---|---|---|---|---|---|---|
| 0 | 1 | 2 | 3 | 4 | 5 | 6 | 7 | 8 | 9 | 10 |
| Not | | | | | | | | | | As bad as |
| at all | | | | | | | | | | it can be |

2. Did you experience discomfort swallowing cold liquids
within the last 24 hours?

| | | | | | | | | | | |
|---|---|---|---|---|---|---|---|---|---|---|
| 0 | 1 | 2 | 3 | 4 | 5 | 6 | 7 | 8 | 9 | 10 |
| Not | | | | | | | | | | As bad as |
| at all | | | | | | | | | | it can be |

3. Did you notice any throat discomfort within the last 24 hours?

| | | | | | | | | | | |
|---|---|---|---|---|---|---|---|---|---|---|
| 0 | 1 | 2 | 3 | 4 | 5 | 6 | 7 | 8 | 9 | 10 |
| Not | | | | | | | | | | As bad as |
| at all | | | | | | | | | | it can be |

4. Did you suffer from muscle cramps within the last 24 hours?

| | | | | | | | | | | |
|---|---|---|---|---|---|---|---|---|---|---|
| 0 | 1 | 2 | 3 | 4 | 5 | 6 | 7 | 8 | 9 | 10 |
| Not | | | | | | | | | | As bad as |
| at all | | | | | | | | | | it can be |

5. Did you experience any other side effects from the chemotherapy
treatment that we did not mention?
No Yes
If Yes, please list:

### Example 4. SapC-DOPS (BXQ-350) Protects Neurons from Oxaliplatin and Promotes the Formation of Neurites

The neurostimulatory, neuroprotective, and neurotrophic qualities of the SapC-phospholipid formulation (BXQ-350) were tested in vitro in two cell lines, i.e., PC-12 cells (derived from a pheochromocytoma of a rat adrenal medulla) and NS20Y cells (cholinergic cell line derived from a mouse neuroblastoma). Cells were dosed with BXQ-350 alone and in combination with oxaliplatin.

PC-12 cells (ATCC, catalogue number CRL-1721) and NS20Y cells (Millipore Sigma, catalogue number 08062517) were seeded overnight in 24-well plates containing 1×10⁴ cells per well in 1 mL of DMEM (Gibco, catalog number 11965-092) supplemented with 10% FBS and PenStrep. Each agent tested was brought to solution, at its appropriate concentrations, in a dosing media consisting of high glucose, no glutamine, no calcium DMEM (Gibco, catalog number 21068-028) supplemented with 4 mM glutamine, 1 mM calcium gluconate and PenStrep. BXQ-350 was tested at concentrations between 5 nM and 1 μM concentration of SapC, and oxaliplatin was tested at concentrations between 100 nM and 4 μM. The seeding medium was completely removed from the cells before the dosing medium containing the testing agents was added.

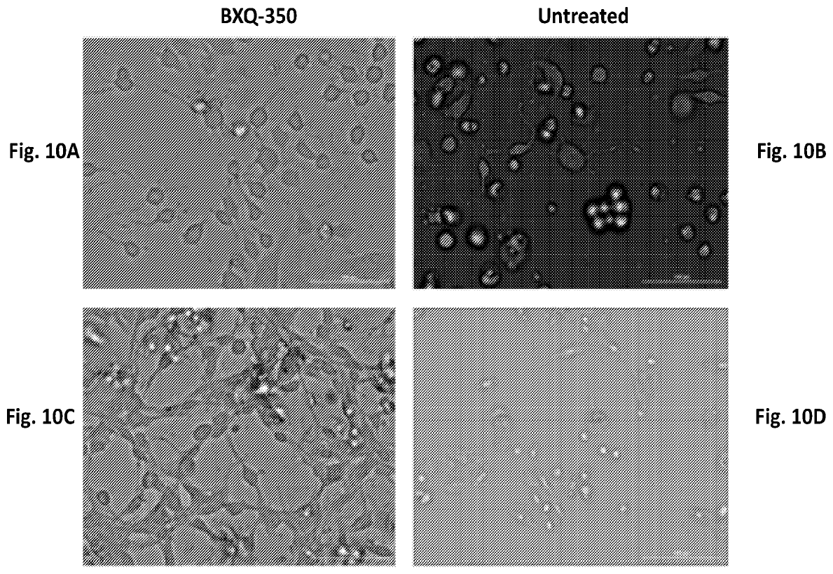

Neurostimulation: BXQ-350 has both neurogenic and neurotrophic activity in both cell lines tested. NS20Y (FIG. 10A) and PC-12 (FIG. 10C) cells treated with 50 nM BXQ-350 produced more cells and a higher percentage of cells with a neuronal morphology when compared with NS20Y and PC-12 cells grown in the control media (FIGS. 10B and 10D, respectively).

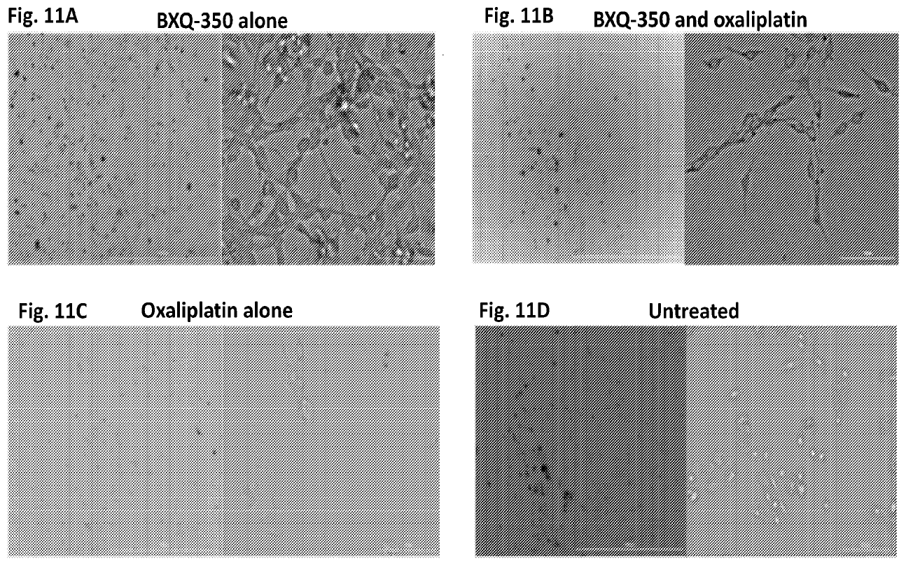

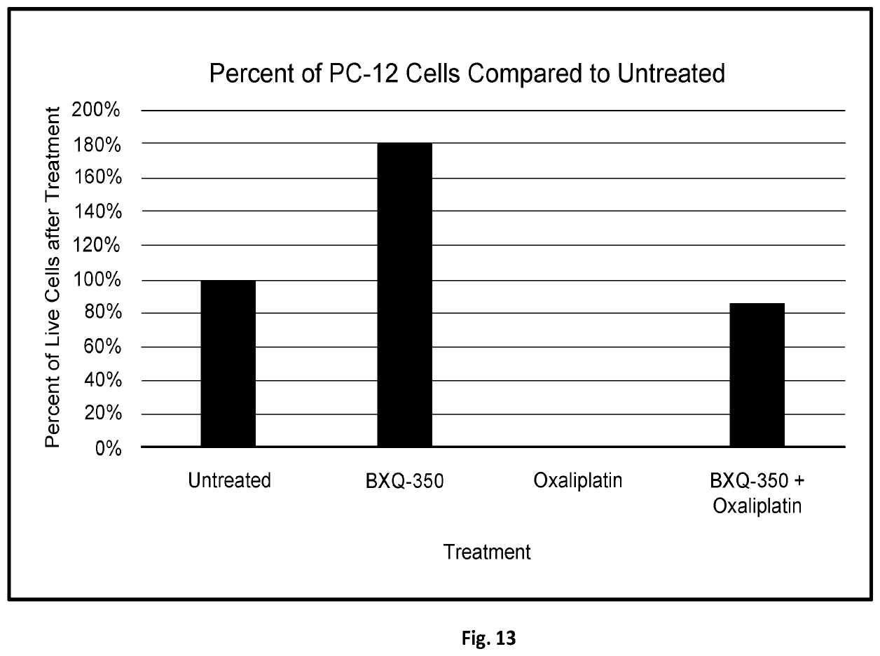

Neuroprotection: BXQ-350 has neuroprotective activity in PC-12 cells when challenged with oxaliplatin, a known cytotoxic drug with neuropathic effects. A 2 μM dose of oxaliplatin severely reduces cell viability (FIG. 13) and may inhibit neurite outgrowth (FIG. 11C), though too few viable cells remain to accurately calculate the percentage of cells with neurites. Cells exposed to a combination of 2 μM oxaliplatin and 50 nM BXQ-350 demonstrated both a preservation of cell viability (FIG. 13) and an increase in neurite outgrowth or neuritogenesis (FIG. 11B), compared to cells treated with oxaliplatin alone (FIG. 13 and FIG. 11C). The images on the left of each pair in FIG. 11 are at 4× magnification, while the images on the right of each pair in FIG. 11 are at 20× magnification.

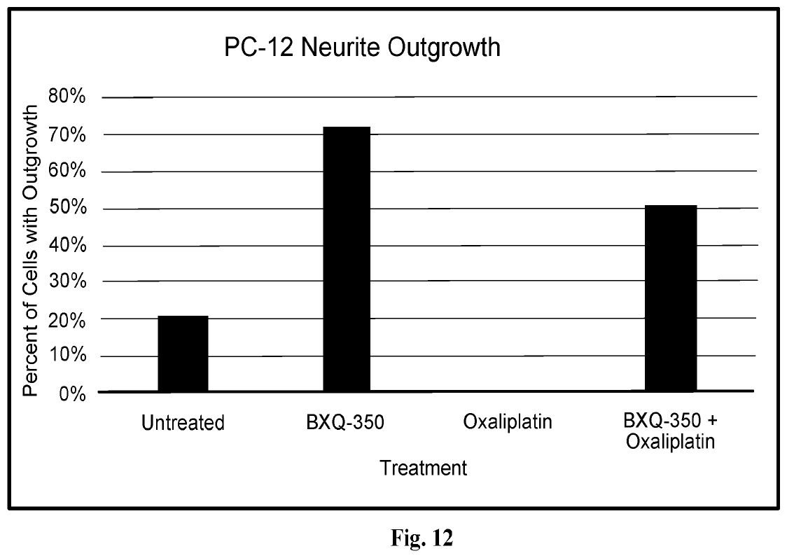

Neuritogenesis: BXQ-350 was shown to stimulate neurite outgrowth in PC-12 cells. Images of the cells were taken on the Biotek Cytation™ 5. Using Biotek Gen5 (ver. 3.05) software, 4× magnification images were analyzed for neurite outgrowth by determining the percentage of cells that had neurite growth at least 1 mm longer than the diameter of the cell body. This analysis of cellular neurite outgrowth (FIG. 12) shows that cells treated with the combination of oxaliplatin/BXQ-350 had a significantly higher percentage of cells with neurite outgrowth than untreated control cells. In fact, the percentage of cells with neurite outgrowth in the oxaliplatin/BXQ-350 treated cells (FIG. 11B) was closer to the percentage with neurite outgrowth in the cells treated with BXQ-350 alone (FIG. 11A) than to the percentage with neurite outgrowth in the untreated cells (FIG. 11D).

Taken together, these results provide evidence that BXQ-350 has both neurogenic and neurotrophic effects, and can provide both neuronal protection and neurite growth stimulation to neuronal cells under neuropathic stress, e.g., from chemotherapy.

### Example 5. SapC-DOPS (BXQ-350) Protects Neurons from Cytotoxic Agents and has Neurostimulatory, Neurotrophic and Neuroprotective Properties

The neurostimulatory, neuroprotective, and neurotrophic qualities of the SapC-phospholipid formulation (BXQ-350) were further tested in vitro in two PC-12 cells and NS20Y cells. Cells were dosed with BXQ-350 alone and in combination with oxaliplatin, vincristine, paclitaxel, or hydrogen peroxide (H₂O₂).

PC-12 cells (ATCC, catalogue number CRL-1721) and NS20Y cells (Millipore Sigma, catalogue number 08062517) were seeded overnight in 24-well plates containing 1×10⁴ cells per well in 1 mL of DMEM (Gibco, catalog number 11965-092) supplemented with 10% FBS and PenStrep. Each agent tested was brought to solution, at its appropriate concentrations, in a dosing media consisting of high glucose, no glutamine, no calcium DMEM (Gibco, catalog number 21068-028) supplemented with 4 mM glutamine, 1 mM calcium gluconate and PenStrep. BXQ-350 was tested at concentrations between 5 nM and 1 μM concentration of SapC. The seeding medium was completely removed from the cells before the dosing medium containing the testing agents was added.

PC-12 and NS20Y cells were dosed with BXQ-350 alone or in combination with oxaliplatin, vincristine, or paclitaxel. Oxaliplatin was tested at concentrations between 100 nM and 4 μM; vincristine was tested between 100 nM-100 μM; and paclitaxel was tested between 100 nM-20 μM. For combination testing, concurrent dosing was done with 50 nM BXQ-350 and 100 nM-4 μM oxaliplatin or 100 nM-10 μM vincristine or 1 μM-10 μM paclitaxel for 72 hours. Images were taken on the Biotek Cytation™ 5 cell imaging system after full exposure to the compounds. Biotek Gen5 (ver. 3.05) software was used to determine neurite outgrowth, which was defined as tendril growth over 1 mm longer than the cell body. Cell counts were determined from these images with untreated cells as a baseline.

PC-12 and NS20Y cells were dosed with 50 nM BXQ-350 alone or left untreated for 72 hours. After 72 hours, the cells were washed with ice-cold PBS and dosed with 200 μM of H₂O₂ for 24 hours. Images were taken on the Biotek Cytation™ 5 cell imaging system after full exposure to the compounds. Biotek Gen5 (ver. 3.05) software was used to determine neurite outgrowth, which was defined as tendril growth over 1 mm longer than the cell body. Cell counts were determined from these images with Untreated cells as a baseline.

As a single agent treatment, BXQ-350 demonstrated neurotrophic properties, induced neurogenesis, neuritogenesis, and was neuroprotective.

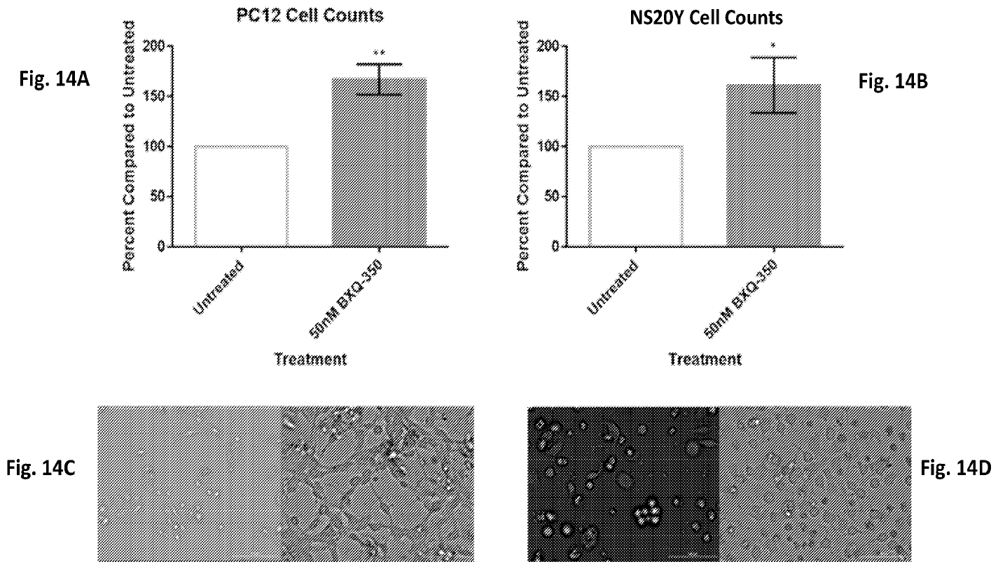

Neurostimulation: BXQ-350 has both neurogenic and neurotrophic activity in both cell lines tested. NS20Y (FIG. 14B) and PC-12 (FIG. 14A) cells treated with 50 nM of BXQ-350 produced approximately 60% more cells than untreated controls, Further, BXQ-350 resulted in markedly more neurite outgrowth in NS20Y pre-neuron cells (FIG. 14D) and PC-12 cells (FIG. 14C), indicating that BXQ-350 stimulates pre-neuron cell lines to both divide and differentiate.

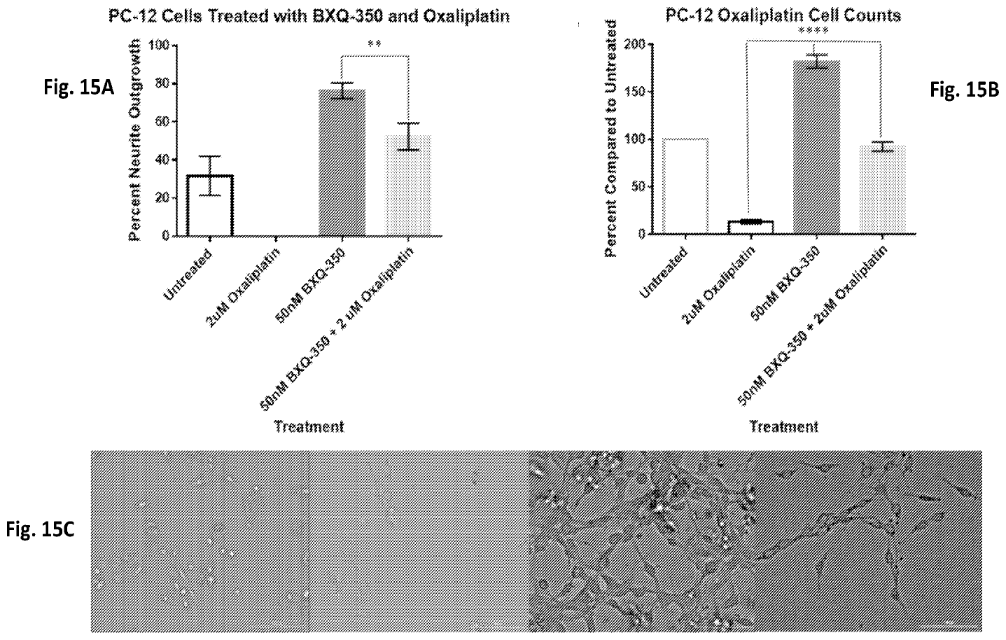

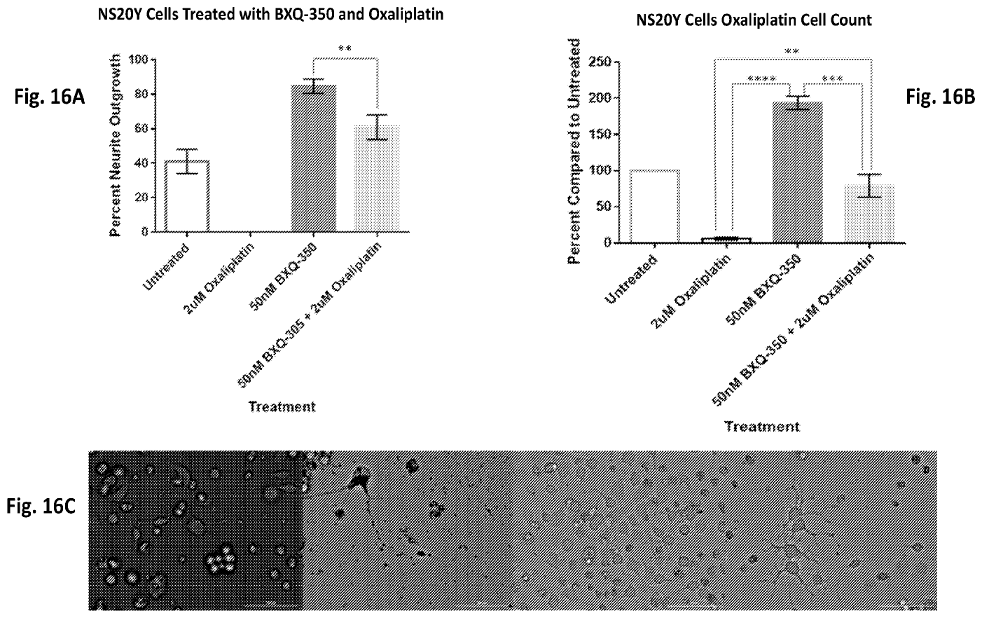

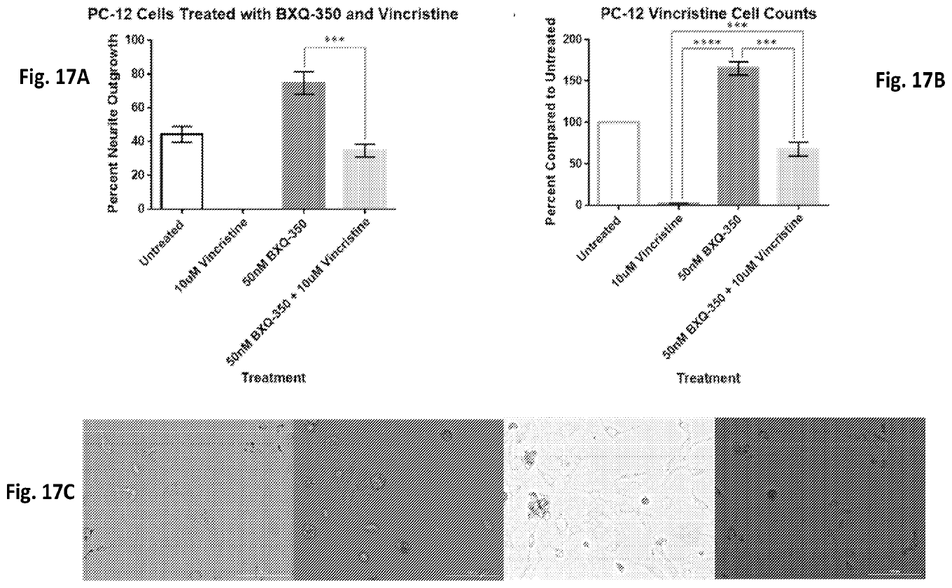

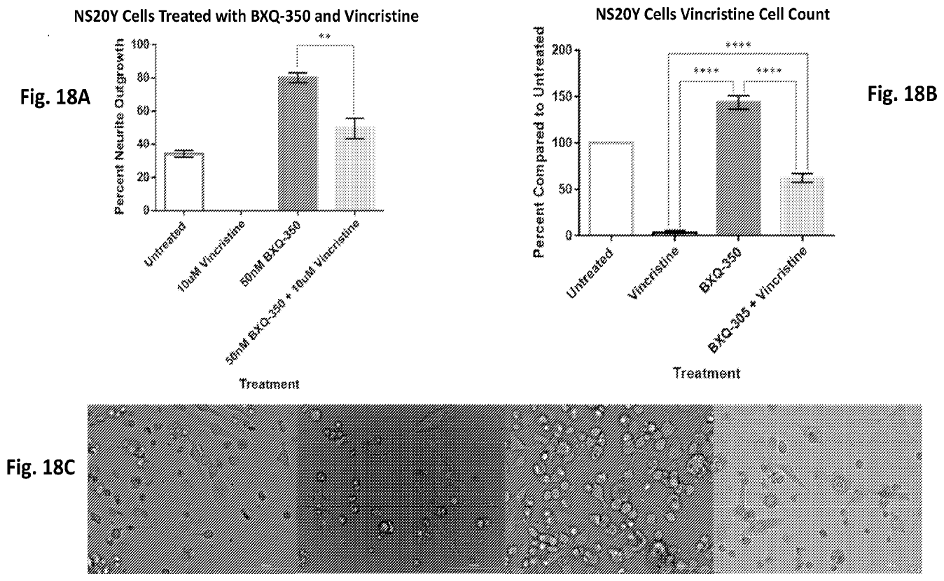

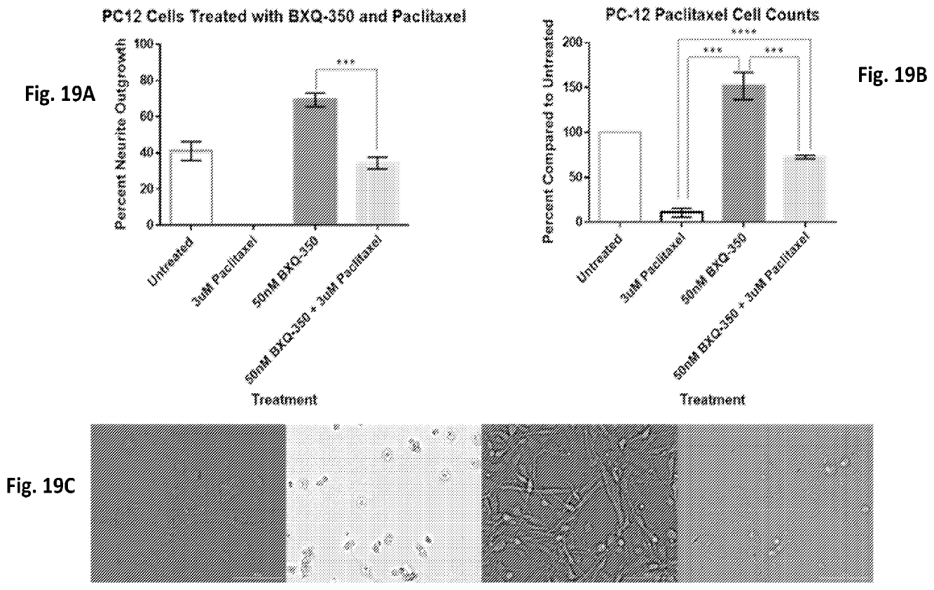

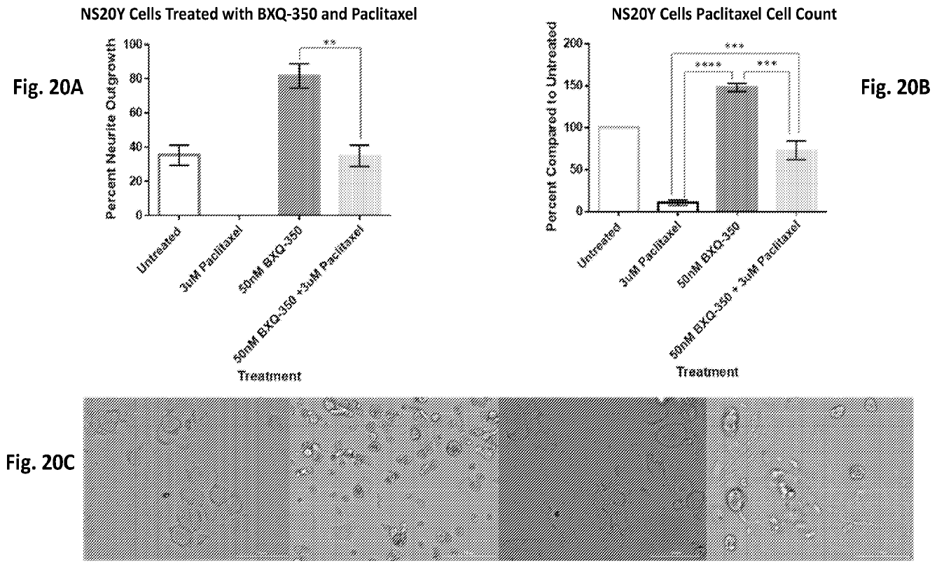

Neuroprotection: BXQ-350 has neuroprotective activity in PC-12 cells when challenged with oxaliplatin, vincristine, or paclitaxel, known cytotoxic drugs with neuropathic effects. PC-12 and NS20Y cells were concurrently dosed with 50 nM BXQ-350 plus either 2 μM oxaliplatin, 10 μM vincristine, or 3 μM paclitaxel for 72 hours. Untreated cells and cells dosed with single agent treatments of 50 nM BXQ-350, 2 μM oxaliplatin, 10 μM vincristine, and 3 μM paclitaxel were included. Massive cytotoxicity was observed among the cells dosed with oxaliplatin (FIGS. 15B and 16B), vincristine (FIGS. 17B and 18B) and paclitaxel (FIGS. 19B and 20B) alone with viability well below 10% and approaching zero. When BXQ-350 was added to any cytotoxic drug, cell viability greatly increased. The viability of the cytotoxic drug+BXQ-350 combination groups was about 50% of the BXQ-350 alone group.

Neuritogenesis: BXQ-350 was shown to stimulate neurite outgrowth in PC-12 cells and NS20Y cells. Images of the cells were taken on the Biotek Cytation™ 5 cell imaging system. Using Biotek Gen5 (ver. 3.05) software, 4× magnification images were analyzed for neurite outgrowth by determining the percentage of cells that had neurite growth at least 1 mm longer than the diameter of the cell body. This analysis of cellular neurite outgrowth shows that cells treated with the combination of oxaliplatin/BXQ-350 (FIGS. 15A and 16A), vincristine/BXQ-350 (FIGS. 17A and 18A), and paclitaxel/BXQ-350 (FIGS. 19A and 20A) had a significantly higher percentage of cells with neurite outgrowth than control cells. 75-80% of the cells dosed with BXQ-350 alone had neurite outgrowth, while 35-50% of the untreated cells and 35-60% of the cytotoxic agent-BXQ-350 combination treated cells had neurites. The oxaliplatin, vincristine and paclitaxel alone groups were unable to be analyzed for neurite outgrowth because the cytotoxicity was so high that an acceptable sample size could not be obtained.

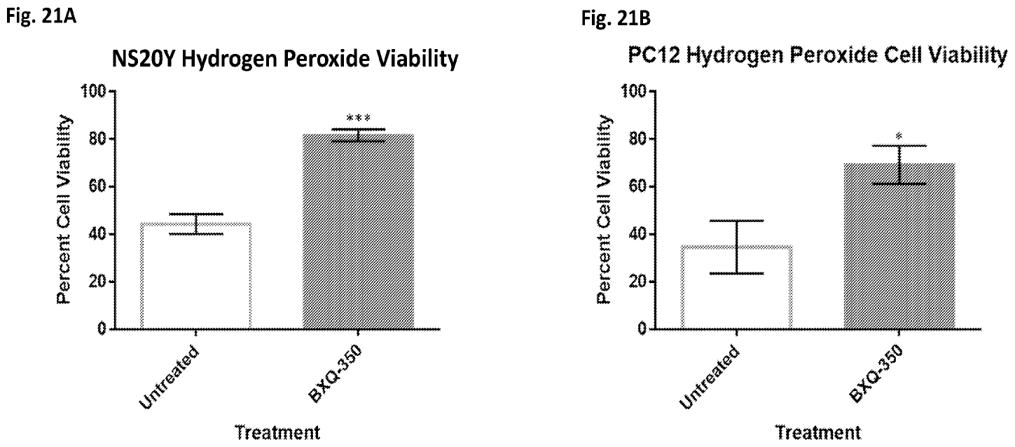

Next, BXQ-350 was dosed in combination with the neurotoxic agent hydrogen peroxide (H₂O₂). PC-12 and NS20Y cells were either pretreated with 50 nM BXQ-350 or left untreated for 72 hours. The media (containing drug or not) was then removed and media containing 200 μM H₂O₂ was added to all groups for 24 hours. For PC-12 cells, only about 35% of the cells were still viable in the untreated group, while the BXQ-350 pretreated group had about 74% viability (FIG. 21B). For NS20Y cells, about 40% of the cells were viable, while about 81% of the BXQ-350 pretreated cells were viable (FIG. 21A).

Taken together, these data suggest that BXQ-350 can simultaneously stimulate neurogenesis cell division, promote neurite outgrowth, and display neuroprotection when exposed to chemotherapeutic agents. Further, these data indicate that BXQ-350 has the potential to protect against a variety of neurotoxic agents. These data also demonstrate that BXQ-350 has the ability to promote neuron growth and neurite development, in vitro. Furthermore, when combined with a general or chemotherapeutic neurotoxic agent, BXQ-350 can exhibit significant neurotrophic activity.

### Example 6. SapC-DOPS Enhances the Cytotoxic Effect of Oxaliplatin and 5-Fluoracil in the HT-29 Colorectal Cancer Cell Line

The ability of a SapC-phospholipid formulation (BXQ-350) to enhance the cytotoxic effect of FOLFOX standard of care drugs oxaliplatin and 5-fluorouracil (5-FU) was tested in an in vitro cancer cell line.

HT-29 cells were plated in 96 well plates at 1×10⁵ cells per well and 100 μL of DMEM (Gibco, catalogue number 11965-092) supplemented with 10% FBS and PenStrep overnight. Blank wells were included, which contained DMEM only. The next day BXQ-350 lyophilized powder was resuspended in a special dosing media consisting of high glucose, no glutamine, no calcium DMEM (Gibco, catalogue number 21068-028) supplemented with 10% FBS, 4 mM glutamine, 1 mM calcium gluconate and PenStrep. The oxaliplatin and 5-FU came in pH controlled solutions. The desired testing concentrations, of either single agent or combination dosing, were created by diluting aliquots of the stock solutions into dosing media.

The growth medium was removed from the culture plates, and the cells were treated with one of the following: single agent; combinations of BXQ-350 with oxaliplatin, BXQ-350 with 5-FU, or 5-FU with oxaliplatin; and a combination of all three agents.

To determine the IC50, BXQ-350 was dosed between 8 μM and 40 μM, with optimal BXQ-350 being between 12 μM to 16 μM. Oxaliplatin was dosed between 0.2 μM to 1 μM, and 5-FU was dosed between 12 μM and 24 μM. Each plate contained untreated cells as a control to compare with the treated cells. The plates were incubated at 37° C., 5% CO₂ for 72 hours. After 72 hours, 10 μL of MTT salt solution was added to each well and the plates were placed back in the incubator for 2 hours. The medium was then removed from each well and replaced with 100 μL of DMSO. The plates were then incubated, as above, overnight. The plates were read on the BioTek Synergy Plate Reader to measure absorbance at 570 nm for data analysis. Cell viability was determined by first subtracting out the blank OD570 value from all wells, then dividing the OD570 value of the dosed cells by the OD570 value of the untreated cells and multiplying by 100. This provides the percent of viable cells in the treated samples using the untreated control as a baseline of 100% viability. At 16 μM, BXQ-350 was lethal as a single agent, but surprisingly, a lower, sublethal concentration of 12 μM BXQ-350 for 72 hours enhanced the cytotoxic effect of oxaliplatin and/or 5-FU.

Using the optimized dosing and MTT cell viability assay (Roche), percent cell viability was determined in treated and untreated HT29 cells. BXQ-350, 5-FU, and oxaliplatin were used at concentrations of 12 μM, 12 μM and 0.3 μM respectively. All combinations were treated concurrently for 72 hours while BXQ-350 was administered by itself for 72 hours. All treatments were done in triplicate.

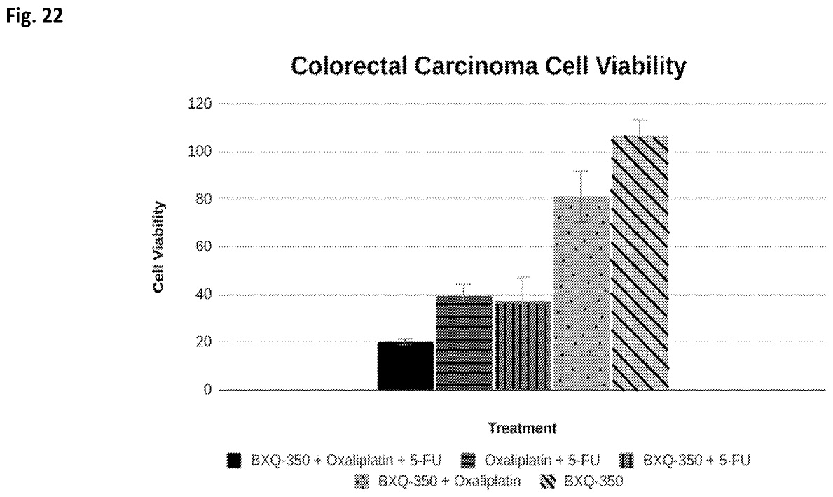

The combination of the three treatments (BXQ-350+oxaliplatin+5-FU) shows significantly higher cytotoxicity (decreased % cell viability) compared to any of the dual combinations (FIG. 22). BXQ-350 with oxaliplatin shows little cytotoxicity compared to the triple combination which shows very good lethality. A doubling in cytotoxicity is seen in the triple combination verses the standard of care oxaliplatin with 5-FU or BXQ-350 with 5-FU.

### Conclusion

These results show that BXQ-350 can significantly enhance the cytotoxic effects of oxaliplatin and/or 5-FU in a cancer cell line.

### Example 7. SapC-DOPS and FOLFOX are Differentially Cytotoxic in Various Colorectal Carcinoma Cell Lines

The combination effect of SapC-DOPS with 5-fluorouracil (5-FU) or oxaliplatin or FOLFOX (5-FU plus oxaliplatin) on cytotoxicity of in vitro cancer cell lines was tested. Human colorectal adenocarcinoma cell lines were obtained from the American Type Culture Collection (ATCC), including SW480 (Catalog #: CCL-228), SW620 (Catalog #: CCL-227, HT29 (Catalog #: HTB-38), HCT116 (Catalog #: CCL-247, LoVo (Catalog #:CCL229), and SW48 (Catalog #: CCL231). The HT29, HCT116, LoVo, and SW48 cells were cultured in High Glucose DMEM (Thermofisher-Gibco, Cat #: 11965-092) supplemented with 9% fetal bovine serum and 1% penicillin-streptomycin. The SW480 and SW620 cells were cultured in either L-15 (ATCC, Cat #: 30-2008) or Leibovitz L-15 (Alphabioregen, Cat #: PG058) supplemented with 9% fetal bovine serum and 1% penicillin-streptomycin. Cells were cultured at 37° C. in an atmosphere containing 5% CO₂. At approximately 80-90% confluency, trypsin (Thermofisher-Gibco, Cat #: 25200-072) was used to detach cells for assay use or passaging.

### MITT Viability Assay:

On Day 1, MTT assay cells were seeded into 96-well absorbance plates (Falcon, Cat #: 353072) at 1.5×10⁴ cells per well in High Glucose DMEM medium and incubated at 37° C., 5% CO₂ for 24 hrs to allow attachment.

On Day 2, lyophilized SapC-DOPS powder (Lot #: 130061) was resuspended and diluted in Low Calcium Medium (Calcium Free DMEM (Gibco, Cat #: 21068-028) supplemented with 9% FBS, 1% Pen-Strep, 4 mM L-Glutamine, and 0.6 mM calcium chloride). Prior to the study, lyophilized oxaliplatin was suspended in WIFI (Gibco, Lot #: 2085277) and frozen as single use aliquots. Lyophilized 5-FU was suspended in ammonium hydroxide (NH₄OH; Sigma, Lot #: BCCF1716) and frozen as single use aliquots. Reconstituted 5-FU and oxaliplatin were diluted in Low Calcium Medium to 500 μM for further use. The final concentrations and dilution volumes are listed as shown below in Table 19.

TABLE 19
Compound dilutions

| | Reconstituted | Working | Dilution to Presented |
|---|---|---|---|
| Compound | Concentration | Dilution | Concentration |
| SapC-DOPS | 250 μM | 250 μM | Added 60 μL stock SapC-DOPS |
| | | | to 940 μL dosing media for |
| | | | 15 μM treatment |
| Oxaliplatin | 12.6 mM | Brought to | Added 20 μL Working Dilution |
| | (Created 50 μL | 500 μM by adding | Oxaliplatin to 980 μL dosing |
| | single use | 1,210 μL dosing | media for 10 μM treatment |
| | aliquots) | media | |
| 5-FU | 25 mM | Brought to | Added 20 μL Working Dilution |
| | (Created 50 μL | 500 μM by adding | 5-FU to 980 μL dosing media |
| | single use | 2,450 μL dosing | for 10 μM treatment |
| | aliquots) | media | |

Medium from overnight culture was removed and plates were treated concurrently with the compounds listed above by themselves, in dual combination, or in triple combination as SapC-DOPS+FOLFOX (without Leucovorin). All plates were incubated at 37° C., 5% CO₂ for 72 hrs.

On Day 4, 10 uL of MTT labeling reagent (3-[4,5-dimethylthiazol-2-yl]-2,5-diphenyltetrazolium bromide) (Sigma-Aldrich, Cat #: 11465007001) was added to each well. The plates were incubated at 37° C., 5% CO₂ for 2 hours. After the incubation, 100 μL of solution in each well was removed and replaced with 100 μL DMSO. Plates were incubated for 1 hr at room temperature and absorbance for each well was measured at 570 nm using the Biotek Synergy™ H1 Hybrid microplate reader.

### Data Processing:

Percent cell viability is calculated as absorbance from blanked treated wells over absorbance from blanked untreated wells.

$$\% Cell Viability = \frac{( ( average OD 570 ) - ( average blank ) )}{( ( average untreated OD 570 ) - ( average blank ) )} * 100$$

### DMSO MIT Experiment:

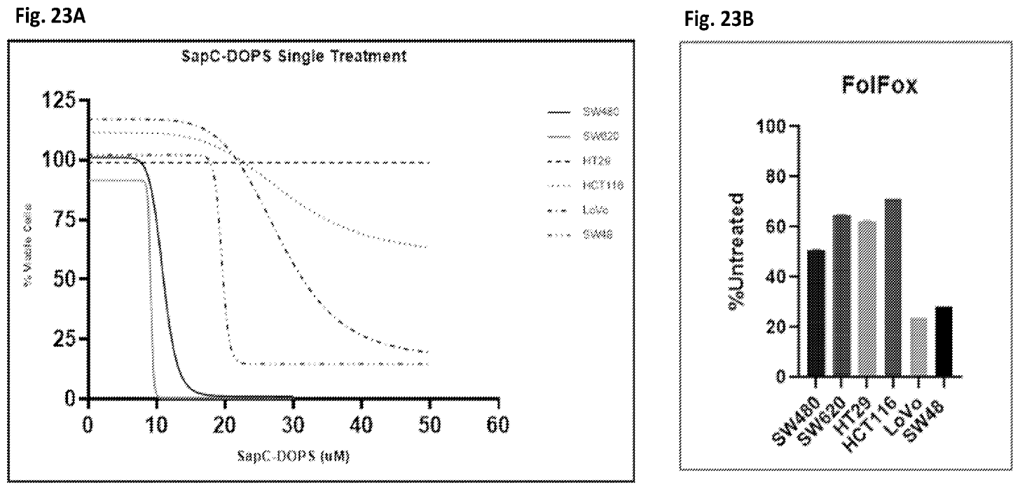

SapC-DOPS, up to 50 μM, and FOLFOX, up to 50 μM, were individually examined for cytotoxicity in all colorectal cancer (CRC) cell lines by DMSO Stop MTT (FIGS. 23A and 23B). 50 μM FOLFOX is equivalent to 50 μM 5-FU and 50 μM Oxaliplatin.

FIG. 23A shows the toxicity curves for each of the 6 CRC cell lines when dosed with SapC-DOPS. SW480 and SW620 cells were dosed at 3, 6, 9, 12, 15, 20, 25, 30 μM SapC-DOPS. HT29, HTC116, LoVo, and SW48 cells were dosed at 5, 10, 20, 30, 40, 50 μM SapC-DOPS. A wide range of responses was seen among the cell lines. SW480 and SW620 cell lines display increased sensitivity to SapC-DOPS compared to previously tested cell lines. An IC50 of between 15-20 μM has been observed in other cell lines, including melanoma, pancreatic and brain/CNS cancer cell lines. In contrast, SW480 and SW620 had IC50s between 9-11 μM (data not shown). LoVo and SW48 had attenuated sensitivity and HT29 and HTC116 appeared to have at least partial resistance to SapC-DOPS.

FIG. 23B displays each cell line's cytotoxic response to one FOLFOX treatment (10 μM Oxaliplatin with 10 μM 5-FU). Each cell line displayed a different response at this concentration of FOLFOX, with two cell lines (LoVo and SW48) comparably more sensitive to FOLFOX treatment than the other lines tested. Since 4 of the 6 cell lines have 50-70% of their cells still viable at 10 μM FOLFOX, this concentration was chosen as a starting point to test synergy with SapC-DOPS.

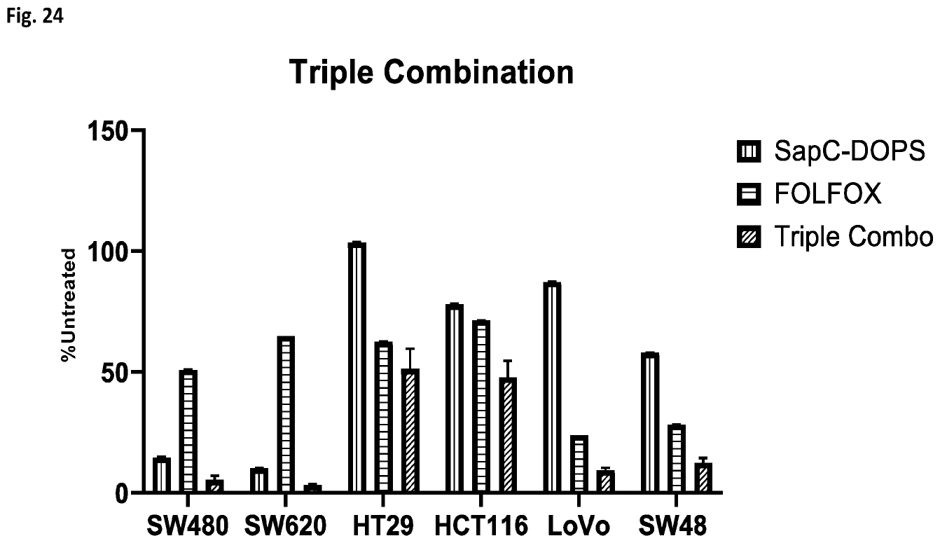

SapC-DOPS was tested in combination with FOLFOX treatment. Several combinations and dosing methods were examined, with the maximum dosing concentration being 25 μM SapC-DOPS and 15 μM FOLFOX. FIG. 24 displays the response of cell lines to one combination of 10 μM FOLFOX and 15 μM SAPC-DOPS, dosed concurrently. Sequential dosing was also performed (data not shown), but no differences were observed compared to concurrent administration. In every cell line, the combination of SapC-DOPS and FOLFOX resulted in lower cell viability than SapC-DOPS alone or FOLFOX alone.

SapC-DOPS and FOLFOX appear to work complementary to each other, which is clinically relevant. The SW480 and SW620 cell lines that showed higher tolerance to FOLFOX were extremely susceptible to SapC-DOPS (see FIGS. 23A and 23B). The LoVo and SW48 cell lines that displayed an attenuated cytotoxicity to SapC-DOPS were very sensitive to FOLFOX treatment (see FIGS. 23A and 23B). This would suggest that the combination of SapC-DOPS and FOLFOX can be used to successfully treat a wider range of CRC tumors than either agent alone. In addition, HT29 and HCT116 cells, which showed relatively high resistance to FOLFOX and SapC-DOPS single agent treatments, had lower cell viabilities when treated with the combination (See FIGS. 23A, 23B, and 24). In instances like this, SapC-DOPS pro-neurotropic abilities can allow patients to tolerate FOLFOX therapy longer until a tumor response can be achieved. Taken together, these data indicate that the combination of SapC-DOPS and FOLFOX is more beneficial than FOLFOX alone.

### Example 8. Repolarization of Macrophages Towards the M1 Phenotype

The ability of a SapC-phospholipid formulation (BXQ-350) to repolarize M2 macrophages towards the M1 phenotype was investigated in an ex vivo immune cell model, using TNF-α as a marker for the M2-phenotype.

Peripheral mononuclear blood cells and macrophages were collected from two healthy human donors (“Donor A” and “Donor B”). CD14⁺ macrophages were separated from the peripheral mononuclear blood cells and checked for purity and viability. The CD14⁺ macrophages then were plated on Day 0 in 384-well plates at 5,000 cells/well density with plating medium. On Day 5, the growth medium was removed from the culture plates, and the cells were treated with BXQ-350 at a range of 2.5 μM to 9 nM in 2-fold serial dilution starting at 2.5 μM. Two reference compounds, prednisolone and dexamethasone, were used as positive controls in separate wells. Aliquots of 10 μM prednisolone and 10 μM dexamethasone were created by diluting the compounds eight times in serial dilutions in each well. An hour after adding BXQ-350 or the positive control compounds, lipopolysaccharide (LPS) at a concentration of 10 ng/ml was added to the cells. As negative controls, LPS-only and LPS+0.1% DMSO were added to separate wells. On Day 8, the cells were fixed with 4% formaldehyde, cells were counted using CD80 stain plus DAPI counterstain, and TNF-α levels were quantified.

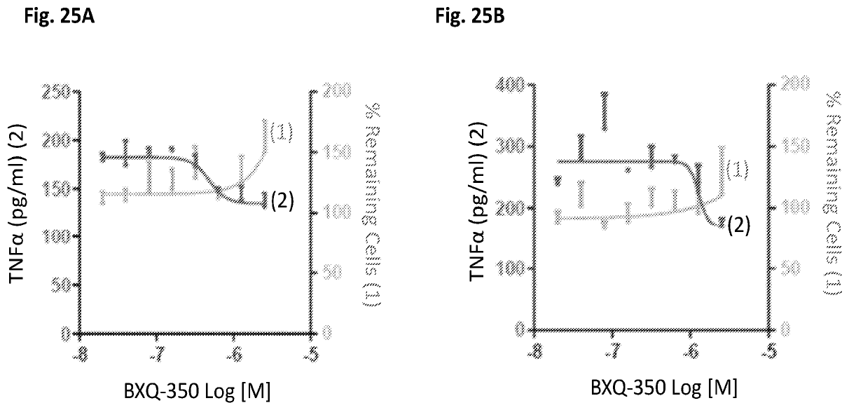

FIGS. 25A and 25B show that addition of BXQ-350 in the range of 2.5 μM to 9 nM produces a concentration-dependent decrease in TNF-α concentration in macrophages obtained from Donor A (FIG. 25A) and Donor B (FIG. 25B). The number of viable cells is unchanged or even increases with addition of BXQ-350, indicating that BXQ-350 is not cytotoxic. BXQ-350 IC₅₀ for repolarization to the M1 phenotype was 0.77 μM and 0.75 μM for the cells from Donor A and Donor B, respectively.

### Conclusion

These results demonstrate that BXQ-350 effectively repolarizes macrophages towards the M1 antitumor phenotype ex vivo.

### Example 9. Activation of T Cells and Associated Killing Effect

As demonstrated in Example 1, BXQ-350 functions to repolarize M2 macrophages towards the M1 phenotype. Whether BXQ-350 also fosters a T-cell mediated anti-tumor immune response was investigated in an ex vivo 3D-tumor spheroid model.

A549 human lung cancer cells, transfected with a lentivirus, were seeded in 96-wells ultra-low attachment plates at a concentration of 750 cells/well. The plates were then centrifuged at 1200 rpm for 2 min, to promote agglomeration of the cells and formation of spheroids. The plates then were incubated for three days at 37° C. After three days, freshly isolated human peripheral blood mononuclear cells (PBMCs) were added to the spheroids at a concentration of 10,000 PBMCs/well. To various wells were added one or more of: an activation cocktail containing an anti-CD3 antibody at a final concentration of 1 ng/ml and IL-12 at a final concentration of 10 ng/ml; BXQ-350 in serial dilutions from 25 μM to 1.27 nM; a positive control of Pembrolizumab at 50 μg/ml. A negative control group had no additions to the spheroids and PBMCs. The final volume of each well was then adjusted to 200 μL per well.

Plates were then left to incubate for an additional 30 minutes before being placed in the Incucyte® live cells analyzer; fluorescence was analyzed every 4 hours for 6 days. Images were then analyzed using the Incucyte® software to determine the IC50. Increased fluorescence intensity in A549 tumor cell spheroids correlates to an increased number of live Luc-A549 cells in the spheroids, and a lack of fluorescence denotes cell death.

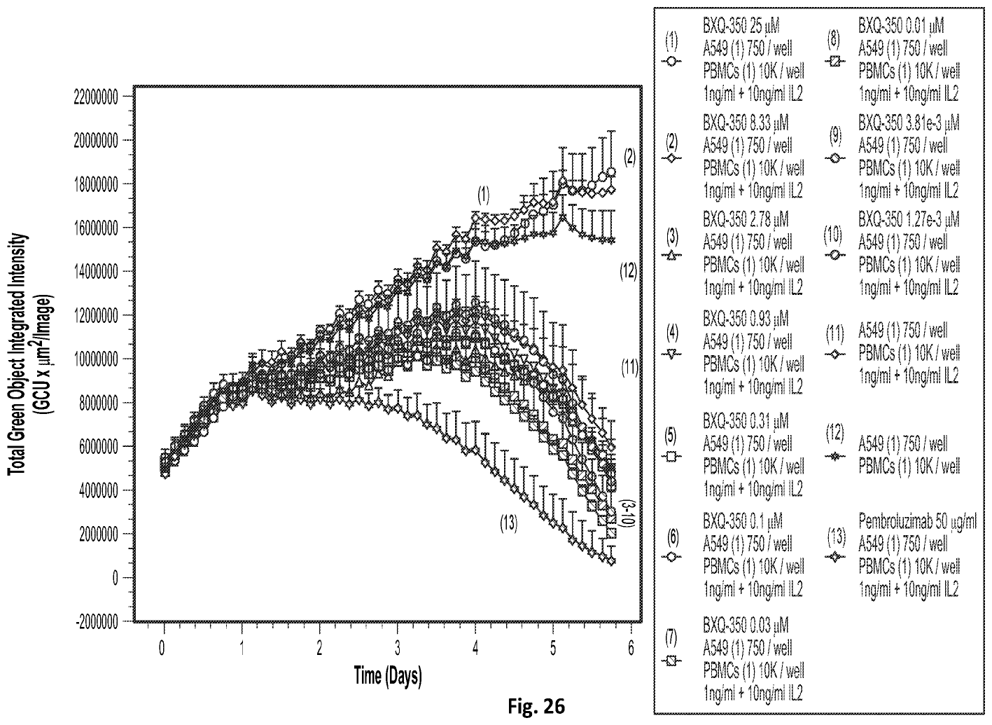

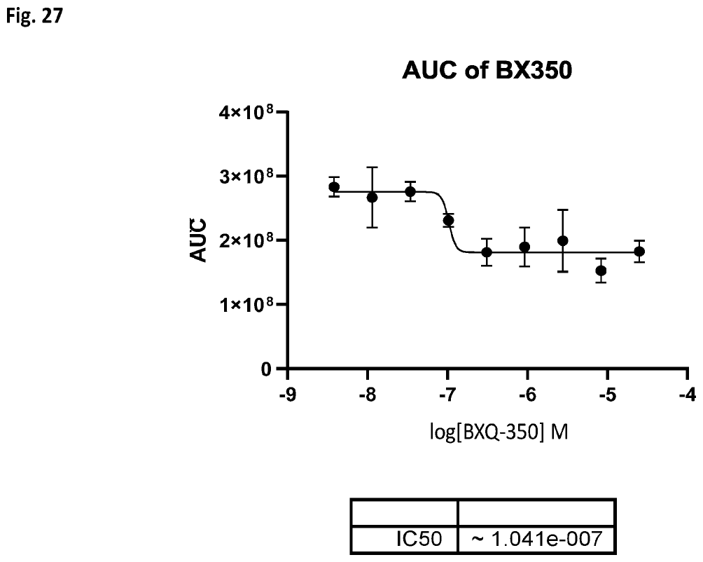

The results of fluorescent intensity of each group of wells as measured over the course of 6 days is shown in FIG. 26. The no activation control group-shown in FIG. 26 (line (12))-showed an increase in intensity over the 6-day study, correlating with increased number of cells over time. Likewise, spheroids treated with 8.33 μM and 25 μM BXQ-350 (lines (1) and (2)) also showed an increase in fluorescent intensity, suggesting that in these experimental conditions a T-cell mediated antitumor immune response was not observed. On the other hand, treatment with Pembrolizumab (line (13)) or with BXQ-350 at concentrations from 1.3 nM to 2.8 μM (lines (3)-(10)) demonstrated increased cell death by day 6, demonstrating that BXQ-350 was more effective at killing cancer cells in this ex vivo 3D-tumor spheroid cytotoxic Tcell model than a T-cell mediated killing effect alone. Finally, using the data in FIG. 26, the area under the curve was calculated to determine the concentration of BXQ-350 necessary for T cell stimulation and enhance their cytotoxic effect. As shown in FIG. 27, the concentration of BXQ-350 necessary for T cell stimulation is 0.104 μM.

### Conclusion

These results demonstrate that BXQ-350, when combined with activated cytotoxic T-cells, effectively stimulates the killing effect of T-cells.

### Example 10. In Vivo Activity of BXQ-350 in a Syngeneic Murine Tumor Model

The activity of BXQ-350 was investigated in a syngeneic CT26 model, a murine colon cancer model that is classified as resistant model that does not respond well to immune check point inhibitors such as an anti-PD1 antibody, an anti-CTLA4 antibody, or an anti-PD-L1 antibody.

Female BALB/c mice 7 to 9 weeks of age were used for this study. The study protocol was approved by the site's Institutional Animal Care and Use Committee (IACUC). Animals were kept in individual cages in housing conditions required by animal welfare guidelines. Diet was also compliant with animal welfare guidelines. As shown in Table 20, the study included four groups of animals treated with: (1) vehicle control; (2) murine SapC-DOPS (m-SapC-DOPS); (3) murine anti-PD1 antibody (m-anti-PD-1) (n=10); and (4) m-SapC-DOPS plus m-anti-PD1 antibody (n=10) as shown in the table below. The m-anti-PD-1 antibody was purchased from BioXcell (Lot No. 780120J2). m-SapC-DOPS was prepared using the procedure described in Qi et al., J Biol Chem, (1996) 22; 271(12):6874-80, which discloses a procedure in which murine SapC was substituted for human SapC.

TABLE 20
Animal Groups

| | | | Dose | Dosing | Dosing | |
|---|---|---|---|---|---|---|
| Group | N= | Treatment | (mg/kg) | Volume | Route | Schedule |
| 1 | 8 | sterile PBS | — | 5 | i.v. | D 0-2, 4, 6, |
| | | | | μL/g | | 8, 10, 14 |
| 2 | 10 | m-SapC- | 12 | 5 | i.v. | D 0-2, 4, 6, |
| | | DOPS | mg/kg | μL/g | | 8, 10, 14, 16 |
| 3 | 10 | m-anti-PD-1 | 10 | 10 | i.p. | D 0, 4, 7, |
| | | | mg/kg | μL/g | | 11, 14 |
| 4 | 10 | m-SapC- | 12 | 5 | i.v./i.p. | D 0-2, 4, 6, |
| | | DOPS + | mg/kg | μL/g + | | 9, 10, 12, 14, |
| | | m-anti-PD-1 | +10 | 10 | | 16 & D 0, 4, |
| | | | mg/kg | μL/g | | 7, 11, 14 |

CT26 cells were cultured in RPMI-1640 medium supplemented with 10% fetal bovine serum, 1% Antibiotic-Antimycotic solution at 37° C. in an atmosphere of 5% CO₂ in air. Cells growing in an exponential growth phase were harvested and counted for tumor inoculation. Each mouse was inoculated subcutaneously at the right lower flank with CT26 tumor cells (0.3×10⁶/mouse) in 0.1 mL of serum-free RPMI-1640 for tumor development. The animals were randomly assigned into 4 groups using an Excel-based randomization software performing stratified randomization based upon their tumor volumes after tumors reached a minimum 60 mm³ volume. Animals with tumors growing intradermally (ID) or intramuscularly (IM) were excluded from the study.

Animals were checked daily for any effects of tumor growth and treatments on normal behavior such as mobility, food and water consumption, body weight gain/loss (body weights were measured three times per week), eye/hair matting, and any other abnormal effect stated in the protocol. Death and observed clinical signs were recorded.

Tumor size was measured three times per week in two dimensions using a caliper, and the volume was expressed in mm³ using the formula: V=0.5 a×b² where a and b are the long and short diameters of the tumor, respectively. The tumor size was then used for the calculations of T/C and TGI values. T and C are the mean volume of the treated and control groups, respectively, on a given day. The T/C value (in percent) is an indication of antitumor effectiveness. TGI was calculated for each group using the formula: TGI (%) [1−(Ti−T0)/(Vi−V0)]×100; Ti was the average tumor volume of a treatment group on a given day, T0 was the average tumor volume of the treatment group on the first day of treatment, Vi was the average tumor volume of the vehicle control group on the same day as for Ti, and V0 was the average tumor volume of the vehicle group on the first day of treatment.

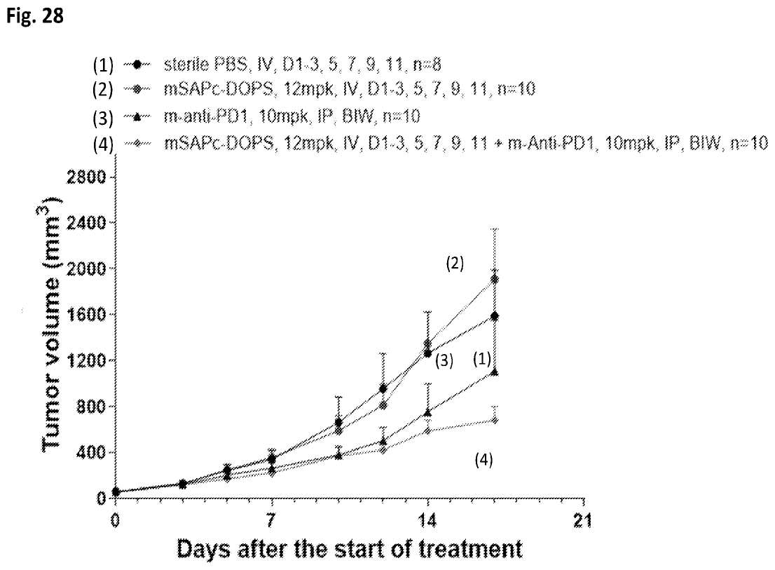

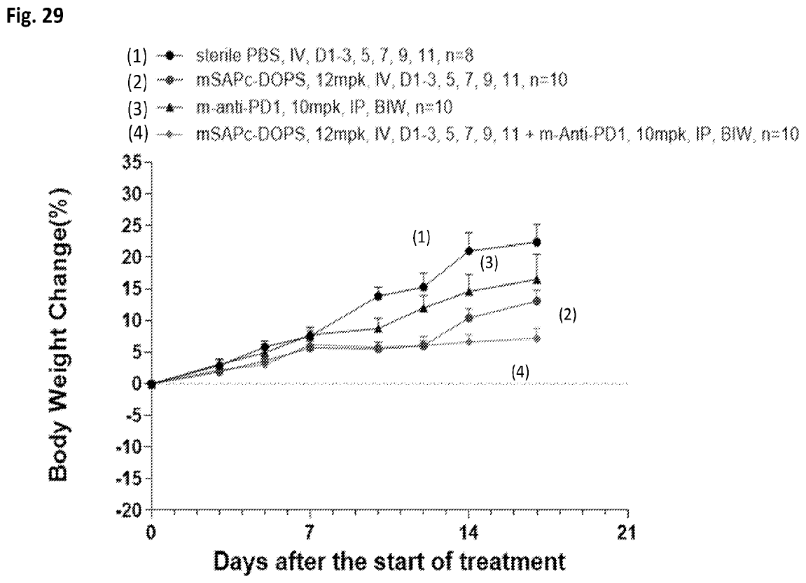

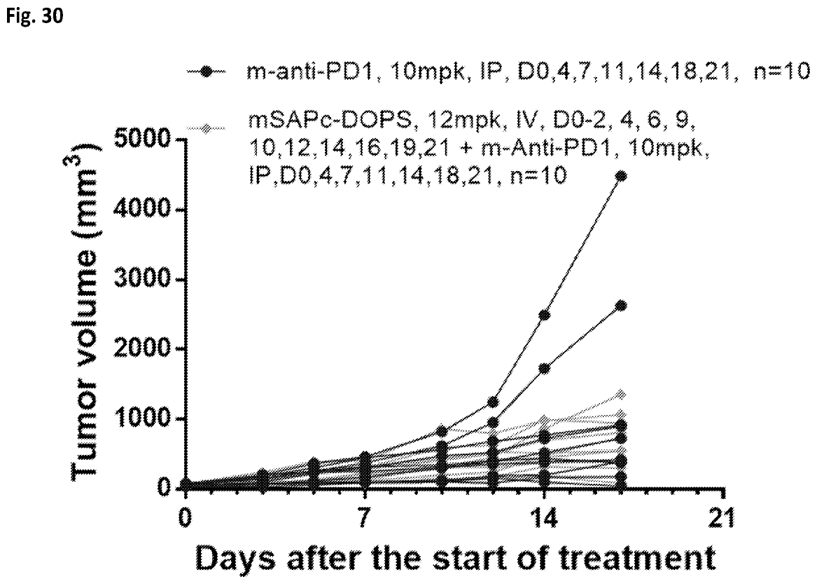

As shown in the Table 21 and FIGS. 28 and 29, the combination of mSapC-DOPS and m-anti-PD1 (lines (4) in FIGS. 28 and 29) results in greater inhibition of tumor growth as well a significantly smaller standard error of the mean (SEM) than observed in the groups that were given m-anti-PD1 alone (lines (3) in FIGS. 28 and 29) or m-SapC-DOPS alone (lines (2) in FIGS. 28 and 29). The lower SEM observed with the combination treatment indicates that more tumors in the combination treatment group than in either of the single-treatment groups responded to the treatment. An additional way to look at these results and the benefit of combining m-SapC-DOPS with m-anti-PD1 is to look at the individual animal tumor growth inhibition plots, shown in FIG. 30. In these plots, one can see that inhibition of tumor growth is noticeably better in the combination group (light shade line) than in the group treated only with m-anti-PD1 (dark shade line). Furthermore, results tabulated in Table 22 indicate that, at Day 17, no tumors in the combination arm were larger then 1600 mm³; in contrast, the two single-treatment arms both had some tumors larger than 1600 mm³ on Day 17: i.e., 3 out of 9 tumors in the m-SapC-DOPS arm and 2 out of 10 tumors in the m-anti-PD1 arm were larger than 1600 mm³.

TABLE 21
Tumor size at Day 17 in Treatment Groups

| | Tumor Size | | |
|---|---|---|---|
| | (mm³)^{a} | T/C^{b} | TGI^{c} |
| Treatment | at day 17 | (%) | (%) |
| Vehicle | 1,589 ± 401 (n = 7) | — | — |
| m-SapC-DOPS (12 mg/kg) | 1,909 ± 436 (n = 9) | 120.13 | −20.9 |
| m-anti-PD1 (10 mg/kg) | 1,110 ± 440 (n = 10) | 69.82 | 31.40 |
| m-SapC-DOPS (12 mg/kg) + | 681 ± 121 (n = 10) | 42.83 | 59.45 |
| m-anti-PD1 (10 mg/kg) | | | |

^{a}Mean ± SEM.
^{b}T/C % = T₁₇/C₁₇ × 100% (T: treatment group; C: control group). T₁₇ is the average tumor volume on Day 17.
^{c}TGI (%) = [1 − (T₁₇ − T₀)/(C₁₇ − C₀)] × 100.
Note:
One animal in Group 1 and one animal in Group 2 were sacrificed on Day 14, so data from the two animals were not included in calculation of Tumor Size, T/C, or TGI value.

TABLE 22
Tumor size at Day 17

| | # of tumors larger than | % of tumors |
|---|---|---|
| | 1600 mm³ at Day 17 | larger than |
| Treatment | (total number of tumors) | 1600 mm³ |
| m-SapC-DOPS | 3 (9) | 33.3 |
| m-anti-PD1 | 2 (10) | 20 |
| m-SapC-DOPS + | 0 (10) | 0 |
| m-anti-PD1 | | |

These results show that a saposin C/phospholipid formulation, SapC-DOPS, plus an immune checkpoint inhibitor is more effective than either agent alone at reducing tumor growth.

### Example 11: Effect of SapC-DOPS on Myeloid Derived Suppressor Cells

Myeloid-derived suppressor cells (MDSCs) are a heterogeneous population of cells that expands during cancer progression. MDSCs have a remarkable ability to suppress T-cell responses, thereby minimizing patients' immune response to cancer. Thus, being able to inhibit the expansion of MDSCs and inhibit their suppressive function is a promising therapeutic approach.

To determine the effect of SapC-DOPS on MDSCs, on Day 0, frozen PBMCs from a healthy donor were thawed in a water bath at 37° C. and washed once with MACS buffer. CD14-positive monocytes were isolated from the preparation of PBMCs using CD14 MicroBeads. The CD14-positive cells were then diluted to 2 million cells/ml with RPMI 1640 culture medium and placed 50 ul/well (0.1 million cells/well) in a flat-bottom 96 well plate. A solution of BXQ-350 at different concentrations was added to the cells, and rhGM-CSF and rhIL-6 were then added into the mix to stimulate monocyte differentiation. The plate was then incubated at 37° C., 5% CO₂ for 7 days.

On Day 3, the medium was replaced with fresh solutions of rhGM-CSF, rhIL-6+IL-2, 1640 medium, and BXQ-350.

On Day 7, about 150 μL of supernatant was taken from the wells and analyzed for IL-10 by ELISA. Also on Day 7, the plate was centrifuged at 350 g for 5 min, the medium was pipetted out, the cells were washed once with 100 μL medium, and a solution of the test compound was added to the wells. T cells isolated from thawed PBMCs using a total T cell Isolation Kit were labeled with a cell trace violet dye, diluted to 2 million/ml, and added to the wells at 50 ul/well (0.1 million T cells/well). CD3/28 Dynabead™ magnetic beads were prepared according to the manufacturer's instruction. The plate was then incubated at 37° C., 5% CO₂ for 4 days.

On Day 11, cells were processed for the MDSC suppression assay. The plate was centrifuged at 300 g for 5 min, and about 150 μL of supernatant from each well was collected for determination of interferon-gamma (IFN-7) levels by ELISA. Then 100 μL Dulbecco's phosphate buffered saline (DPBS) was added to each well of the plate, the mixture was mixed well by gently pipetting multiple times, and the cell/bead suspensions were transferred into a new 96-V well plate. That plate containing the T cell/bead suspensions was placed on a magnet plate for 2 minutes, after which the T cell suspensions were removed from that plate and added into a new 96-well plate. The T cells in the latter plate were then stained with 100 L/well using a Live/Dead™-Near-Infrared (IR) stain at a dilution of 1:1000 in DPBS for 20 minutes in order to determine cell viability. Mixtures were washed once with staining buffer. The cells in different wells were then labeled with antibodies for CD4+ T cells, CD8+ T cells, IL-10, HLA-DR, CD86, CD80, or CD11b and detected via ELISA (e.g., for IL-10) or FACS analysis using a BD FortessaX20™ cell analyzer according to manufacturer's instructions.

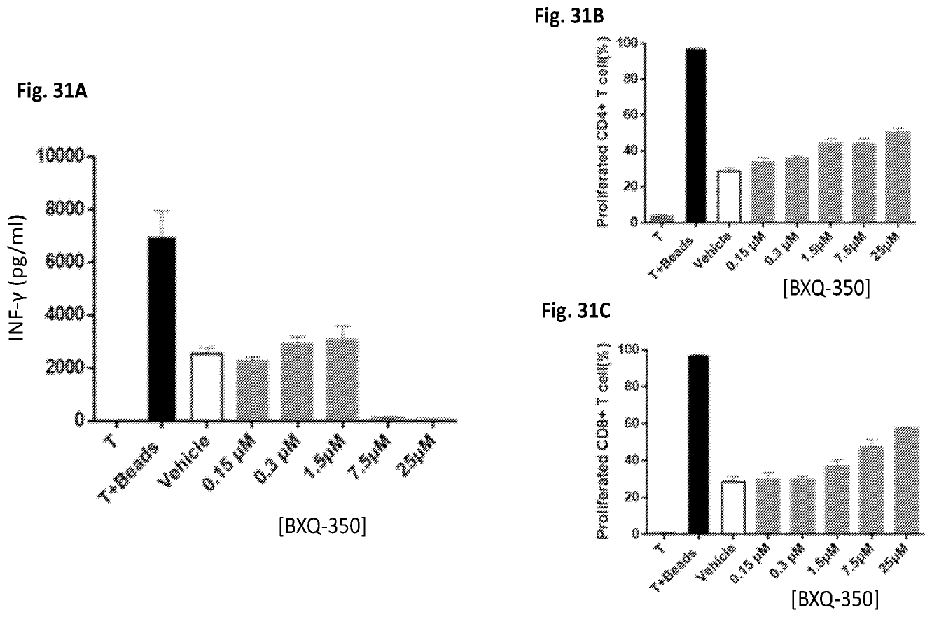

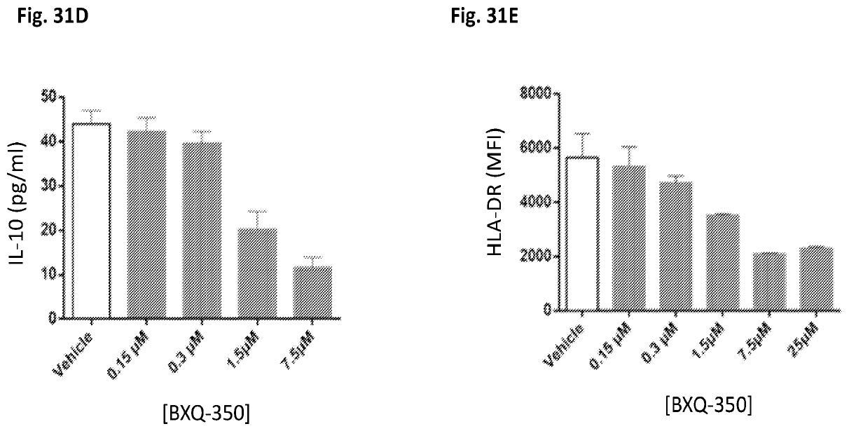

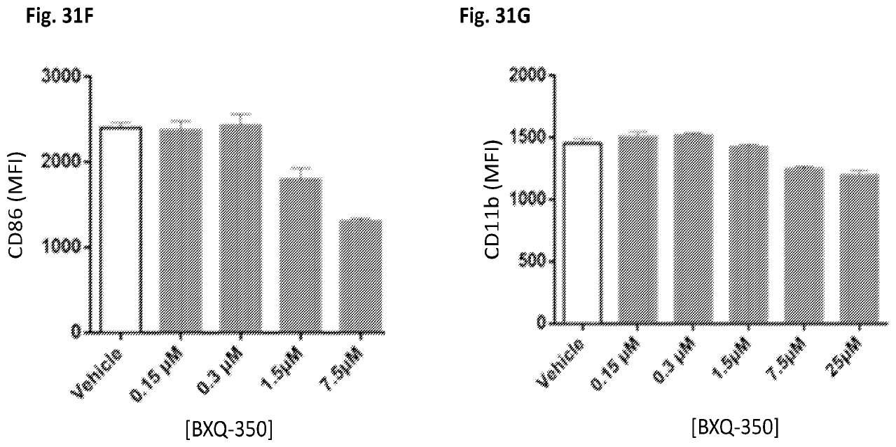

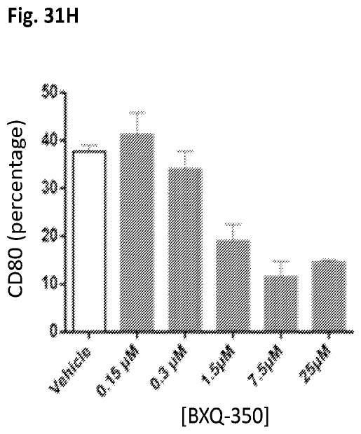

As shown in FIG. 31A, BXQ-350 at concentrations from 0.3 to 1.5 μM promoted expression of IFN-7 in this assay. FIGS. 31B-31C show that proliferation of both CD4+ T cells and CD8+ T cells increased in the presence of BXQ-350, in a dose-dependent manner. Furthermore, BXQ-350 significantly reduces the expression of IL-10 (FIG. 31D), HLA-DR (FIG. 31E), CD86 (FIG. 31F), and CD80 (FIG. 31H), while modestly decreasing the expression of CD11b (FIG. 31G).

### Conclusion

Overall, the data indicate that BXQ-350 has the ability to reverse the suppressive nature of MDSC towards T cells at relatively low concentrations.

### Example 12: Impact of BXQ-350 on the Concentration of Systemic Cytokines in Subjects with Cancer

Cytokines are secreted by specific cells of the immune system and have an effect (1) on other cells, including cells of the immune system, (2) on the expression of complexes of differentiation on immune cells, (3) on the induction of specific immune responses, and (4) on the activation and coordination the overall immune response across different diseases. See Dranoff, Nat Rev Cancer, (2004) 4(1):11-22, which is incorporated by reference in its entirety. Systemic monitoring of the concentration of specific cytokines is necessary to understand the relative changes of specific cytokines over time, and these relative changes are indicative of a response of the immune system.

Monitoring the concentration of specific cytokines over time in cancer patients after a therapeutic intervention (e.g., administration of a pharmaceutical compound; e.g., BXQ-350)) can be indicative of the stimulation of the immune system of these patients towards an immune response that would inhibit cancer progression and even support cancer regression. Further, as markers of cancer regression or progression, cytokines (through their systemic expression) can be identified as having an anti-cancer or pro-cancer effect. For instance, systemic cytokines indicative of an anti-cancer effect include, but are not limited to, interferon-gamma (IFN-7) (see Ikeda, H. et al., Cytokine Growth Factor Rev, (2002) 13(2):95-109); tumor necrosis factor-alpha (TNF-α) (see van Horssen, R. et al., Oncologist (2006); 11(4):397-408); interleukin 2 (IL-2) (Wrangle, J. M. et al., J Interferon Cytokine Res, (2018) 38(2):45-68); interleukin-12 (IL-12); and interleukin-15 (IL-15). Systemic cytokines indicative of a pro-cancer effect include, but are not limited to, interleukin-1 (IL-1); interleukin-6 (IL-6) (Kumari, N. et al., Tumour Biol (2016) 37(9):11553-11572); interleukin-8 (IL-8) (Waugh D. et al. Clin Cancer Res (2008) 14(21):6735-41); and interleukin-10 (IL-10) (Wang X. et al. Cold Spring Harb Perspect Biol. (2019) 11(2):a028548). Each of the references cited in this paragraph is incorporated by reference in its entirety.

A Phase 1 study to determine the anti-cancer effectiveness of BXQ-350 was conducted in adult patients with advanced solid tumors (Clinical Trials.gov Identifier: NCT02859857) who had been previously treated with chemotherapeutic agents. Plasma samples of seven enrolled patients (Patients A-G) were collected over time at pre-dose of BXQ-350 administration (Day 1) and 29 days after. Tables 23-25 show that the concentration of cytokines associated with anti-cancer progression properties (i.e., IFN-7 in Patients A-D; TNF-α in Patients A, C, E, F, and G; and IL-2 in Patients B, D, and F) increased following administration of BXQ-350. Tables 26-28 show that the concentration of cytokines associated with cancer progression (i.e., IL-6 in Patients A, B, D, and G; IL-8 in Patients A, B, C, D, and G; and IL-10 in Patients A, B, E, and G) decreased after administration of BXQ-350. These results indicate that BXQ-350 positively influences and rebalances the immune system towards an antitumor state.

Furthermore, as shown in Table 29, changes in various cytokine concentrations between Day 1 and Day 29 for Patients A and B are an indication that their immune systems are switching to an anti-cancer state. The anti-cancer state correlated with a clinical benefit: Patient A, a glioblastoma multiform cancer patient, lived more than 5 years after initiating BXQ-350 treatment, and Patient B, an appendiceal carcinoma patient, lived more than 2 years after initiating BXQ-350 treatment.

### Conclusion

These results indicate that BXQ-350 positively influences and rebalances the immune system towards an antitumor state.

TABLE 23
Interferon-γ Expression at Day 1 and Day 29

| Patient | A | B | C | D |
|---|---|---|---|---|
| Cytokine | IFN-γ | IFN-γ | IFN-γ | IFN-γ |
| Pro-/Anti- | Anti | Anti | Anti | Anti |
| Cancer | | | | |
| Day 1* | 6.5 | 3.9 | 34.5 | BLQ |
| Day 29* | 10.9 | 9.6 | 55.6 | 4.8 |
| Change | +68% | +146% | +61% | N/A |

*= concentrations in pg/ml of IFN-γ at Day 1 or Day 29
BLQ: Below Limit of Quantitation
N/A: Change not calculated because baseline expression was BLQ

TABLE 24
TNF-α Expression at Day 1 and Day 29

| Patient | A | C | E | F | G |
|---|---|---|---|---|---|
| Cytokine | TNF-α | TNF-α | TNF-α | TNF-α | TNF-α |
| Pro-/Anti- | Anti | Anti | Anti | Anti | Anti |
| Cancer | | | | | |
| Day 1* | 10.6 | 83.2 | 3.9 | BLQ | 15.3 |
| Day 29* | 43.6 | 86.8 | 9.6 | 16.2 | 20.0 |
| Change | +311% | +4% | +146% | N/A | +31% |

*= concentrations in pg/ml of TNF-α at Day 1 or Day 29
BLQ: Below Limit of Quantitation
N/A: Change not calculated because baseline expression was BLQ

TABLE 25
IL-2 Expression at Day 1 and Day 29

| Patient | B | D | F |
|---|---|---|---|
| Cytokine | IL-2 | IL-2 | IL-2 |
| Pro-/Anti- | Anti | Anti | Anti |
| Cancer | | | |
| Day 1* | 1.7 | 1.9 | BLQ |
| Day 29* | 2.2 | 2.4 | 1.9 |
| Change | +29% | +26% | N/A |

*= concentrations in pg/ml of IL-2 at Day 1 or Day 29
BLQ: Below Limit of Quantitation
N/A: Change not calculated because baseline expression was BLQ

TABLE 26
IL-6 Expression at Day 1 and Day 29

| Patient | A | B | D | G |
|---|---|---|---|---|
| Cytokine | IL-6 | IL-6 | IL-6 | IL-6 |
| Pro-/Anti- | Pro | Pro | Pro | Pro |
| Cancer | | | | |
| Day 1* | 10.3 | 26.6 | 8.6 | 46.4 |
| Day 29* | 4.3 | 17.9 | 5.8 | 18.7 |
| Change | −58% | −37% | −32% | −60% |

*= concentrations in pg/ml of IL-6 at Day 1 or Day 29

TABLE 27
IL-8 Expression at Day 1 and Day 29

| Patient | A | B | C | D | G |
|---|---|---|---|---|---|
| Cytokine | IL-8 | IL-8 | IL-8 | IL-8 | IL-8 |
| Pro-/Anti- | Pro | Pro | Pro | Pro | Pro |
| Cancer | | | | | |
| Day 1* | 19.4 | 38.3 | 17.9 | 25.4 | 70.4 |
| Day 29* | 12.5 | 34.7 | 14.9 | 21.8 | 56 |
| Change | −55% | −9% | −17% | −14% | −20% |

*= concentrations in pg/ml of IL-8 at Day 1 or Day 29

TABLE 28
IL-10 Expression at Day 1 and Day 29

| Patient | A | B | E | G |
|---|---|---|---|---|
| Cytokine | IL-10 | IL-10 | IL-10 | IL-10 |
| Pro-/Anti- | Pro | Pro | Pro | Pro |
| Cancer | | | | |
| Day 1* | 4.7 | 1.7 | 1.9 | 1.3 |
| Day 29* | 1.6 | 1.3 | 1.4 | 0.7 |
| Change | −66% | −25% | −26% | −46% |

*= concentrations in pg/ml of IL-10 at Day 1 or Day 29

TABLE 29
Percent Change in Cytokine Expression from Day 1 to Day 29

| | Anti-Cancer Cytokines | | | Cancer Progression Cytokines | | |
|---|---|---|---|---|---|---|
| | Cytokine | | | | | |
| | IFN-γ | TNF-α | IL-2 | IL-6 | IL-8 | IL-10 |
| | Change | | | | | |
| | % Change Day 1 to Day 29 | | | % Change Day 1 to Day 29 | | |
| Patient A | +68% | +311% | — | −58% | −55% | −66% |
| Patient B | +148% | — | +29% | −37% | −9% | −25% |

Whereas particular features of the present invention have been described above for purposes of illustration, it will be evident to those skilled in the art that numerous variations of the details of the methods disclosed herein may be made without departing from the scope in the appended claims.

```
SEQ ID NO: 1
Ser Asp Val Tyr Cys Glu Val Cys Glu Phe Leu Val
1 5 10
Lys Glu Val Thr Lys Leu Ile Asp Asn Asn Lys Thr
                15 20
Glu Lys Glu Ile Leu Asp Ala Phe Asp Lys Met Cys
25 30 35
Ser Lys Leu Pro Lys Ser Leu Ser Glu Glu Cys Gln
                        40 45
Glu Val Val Asp Thr Tyr Gly Ser Ser Ile Leu Ser
        50 55 60
Ile Leu Leu Glu Glu Val Ser Pro Glu Leu Val Cys
                                65 70
Ser Met Leu His Leu Cys Ser Gly
                75 80
```

What is claimed is:

1. A method of reducing a neuropathic symptom in a human subject, the method comprising:
identifying a human subject who is suffering from a neuropathic symptom;
administering to the subject a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH; and
confirming that the subject's neuropathic symptom is reduced.

2. A method of reducing the incidence, intensity, and/or duration of a neuropathic symptom, or delaying onset of a neuropathic symptom, in a human subject in need thereof, the method comprising:
identifying a human subject who is at risk of experiencing a neuropathic symptom; and
treating the subject with a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH,
wherein the treatment is effective in reducing the incidence, intensity, and/or duration of the neuropathic symptom, or delaying onset of the neuropathic symptom in the subject.

3. A method of reducing the incidence, intensity, and/or duration of a neuropathic symptom, or delaying onset of a neuropathic symptom, in a human subject in need of treatment with a chemotherapeutic agent that is associated with neuropathic symptom side effects, the method comprising:
administering a dose of the chemotherapeutic agent to the subject; and
concurrently with the dose of the chemotherapeutic agent, or immediately before or after administering the dose of the chemotherapeutic agent, administering to the subject at least one dose of a nanovesicle formulation comprising (i) a saposin C polypeptide comprising SEQ ID NO: 1 with zero to four amino acid insertions, substitutions, deletions, or combination thereof; and (ii) a phospholipid having a net negative charge at neutral pH.

4. A method of treating cancer, the method comprising co-administering to a human subject in need thereof:
a chemotherapeutic agent that is associated with neuropathic symptom side effects; and
a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH,
wherein the nanovesicle formulation is administered in an amount that reduces the incidence, intensity, and/or duration of the neuropathic symptom side effects associated with the chemotherapeutic agent, or delays onset of the neuropathic symptom side effects associated with the chemotherapeutic agent.

5. A method of reducing a neuropathic symptom in a human subject in need thereof, the method comprising
identifying a subject who is experiencing a neuropathic symptom; and
treating the subject with an amount of a nanovesicle formulation effective to reduce the subject's neuropathic symptom, wherein the nanovesicle formulation comprises (i) a saposin C polypeptide comprising SEQ ID NO: 1 with zero to four amino acid insertions, substitutions, deletions, or combination thereof, and (ii) a phospholipid having a net negative charge at neutral pH.

6. A method of treating gastrointestinal cancer in a subject, the method comprising administering to the subject
(a) a chemotherapeutic regimen comprising one or more antineoplastic agents, and
(b) a nanovesicle formulation comprising (i) a saposin C polypeptide comprising SEQ ID NO: 1 with zero to four amino acid insertions, substitutions, deletions, or combination thereof; and (ii) a phospholipid having a net negative charge at neutral pH.

7. The method of any one of claims 1-6, wherein the saposin C polypeptide's amino acid sequence comprises SEQ ID NO: 1 with one or two amino acid insertions, substitutions, deletions, or combination thereof.

8. The method of any one of claims 1-6, wherein the saposin C polypeptide's amino acid sequence comprises SEQ ID NO: 1.

9. The method of any one of claims 1-6, wherein the saposin C polypeptide's amino acid sequence consists of SEQ ID NO: 1.

10. The method of any one of claims 1-9, wherein the phospholipid comprises phosphatidylserine.

11. The method of any one of claims 1-9, wherein the phospholipid comprises dioleoyl phosphatidylserine (DOPS) or a salt thereof.

12. The method of claim 11, wherein the phospholipid comprises a sodium salt of DOPS.

13. The method of any one of claims 1-9, wherein the phospholipid comprises phosphoglyceride.

14. The method of any one of claims 1-11, wherein the phospholipid comprises one or more of dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, or diphytanoyl phosphatidylserine lipid.

15. The method of claim 13, wherein the phosphoglyceride comprises phosphatidate.

16. The method of any one of claims 1-15, wherein the molar ratio of the phospholipid to the saposin C polypeptide in the nanovesicle formulation is in the range of 8:1 to 20:1.

17. The method of any one of claims 1-15, wherein the molar ratio of the phospholipid to the saposin C polypeptide in the nanovesicle formulation is in the range of 11:1 to 13:1.

18. The method of any one of claims 1-15, wherein the molar ratio of the phospholipid to the saposin C polypeptide in the nanovesicle formulation is 12:1.

19. The method of any one of claims 1-5, wherein the subject has cancer.

20. The method of claim 19, wherein the subject has gastrointestinal cancer, pancreatic cancer, colorectal cancer, bone cancer, brain cancer, sarcoma, neuroblastoma, breast carcinoma, or squamous cell carcinoma.

21. The method of any one of claims 1, 2, and 5, wherein the subject has or is at risk of having peripheral neuropathy.

22. The method of claim 3 or 4, wherein the subject has or is at risk of having peripheral neuropathy.

23. The method of claim 21 or 22, wherein the subject has or is at risk of having chemotherapy-induced peripheral neuropathy (CIPN) that is induced by one or more chemotherapeutic agents.

24. The method of claim 23, wherein the chemotherapeutic agent is selected from the group consisting of a platinum-based agent, a taxane, an epothilone, a plant alkaloid, an immunomodulatory agent, and a proteasome inhibitor.

25. The method of claim 23, wherein the chemotherapeutic agent is oxaliplatin, cisplatin, carboplatin, paclitaxel, docetaxel, cabazitaxel, ixabepilone, vinblastine, vincristine, vindesine, vinorelbine, vincaminol, vineridine, vinburnine, etoposide, thalidomide, lenalidomide, pomalidomide, bortezomib, carfilzomib, ixazomib, eribulin or suramin.

26. The method of claim 21 or 22, wherein the subject has or is at risk of having oxaliplatin-induced acute pain syndrome.

27. The method of any one of claims 1, 2, and 5, wherein the subject has or is at risk of having neuropathy associated with one or more of the following conditions: amyotrophic lateral sclerosis (ALS), spinal muscular atrophy (SMA), rheumatoid arthritis, systemic lupus erythematosus (SLE), post-polio syndrome, Guillain-Barre syndrome, chronic inflammatory demyelinating polyneuropathy (CIDP), multifocal motor neuropathy, muscular dystrophy, peripheral nerve injuries, demyelination, acute disseminated leukoencephalitis, progressive multifocal leukoencephalitis (PML), adrenal leukodystrophy, optic neuritis, kidney disease, liver disorder, transverse myelitis, Parkinson's disease, stroke, Alzheimer's disease, Lyme disease, carpal tunnel syndrome, lymphoma, neuroma, multiple myeloma, vitamin B12 deficiency, post-herpetic neuralgia, leprosy, Charcot-Marie-Tooth disease, Fabry disease, critical illness polyneuropathy, Bell's palsy, ulnar nerve palsy, and peroneal nerve palsy.

28. The method of any one of claims 1, 2, and 5, wherein the subject has or is at risk of having neuropathy associated with diabetes or a viral infection.

29. The method of any one of claims 1-28, wherein the nanovesicle formulation is administered intravenously, intra-arterially, intradermally, intramuscularly, intra-cardiacally, intracranially, subcutaneously, intraperitoneally, inhalationally, nasally, orally, or sublingually.

30. The method of any one of claims 1-29, wherein each dose of the nanovesicle formulation administered to the subject contains 0.4 mg/kg to 7 mg/kg of the saposin C polypeptide.

31. The method of claim 30, wherein the nanovesicle formulation is administered intravenously.

32. The method of claim 30 or 31, wherein the nanovesicle formulation is administered at least once a day, once every 2 days, 3 times a week, approximately once every week (every 7 (+/−3) days), or approximately once every 2 weeks (every 14 (+/−3) days).

33. The method of claim 32, wherein multiple doses of the nanovesicle formulation are administered over a period of at least 8 weeks.

34. The method of any one of claims 1-5, wherein the neuropathy or neuropathic symptom is confirmed to be reduced as measured using one or more assessment tools selected from the group consisting of the National Cancer Institute-Common Toxicity Criteria (NCI-CTC), the Numeric Rating Scale (NRS), the Visual Analog Scale (VAS), the European Organization for Research and Treatment of Cancer (EORTC) Qualify of Life (QLQ)-CIPN20, QLC-C30, the Functional Assessment of Cancer Therapy/Gynecologic Oncology Group-Neurotoxicity (FACT/GOG-Ntx), the Total Neuropathy Score (TNS) questionnaire, the Chemotherapy-Induced Peripheral Neuropathy Assessment Tool (CIPNAT), and the Post-Oxiplatin Symptom Survey.

35. The method of any one of claims 1-5, wherein the reduction in neuropathy or neuropathic symptom comprises a change in at least one parameter selected from a reduction in the incidence of neuropathy, a reduction in the intensity of neuropathy, a reduction in the duration of neuropathy, a reduction in the duration of neuropathic episodes, a reduction in the frequency of neuropathic episodes, and delaying onset of neuropathy.

36. The method of claim 1, wherein the reduction in neuropathy comprises a reduction in a neuropathic symptom.

37. The method of claim 2 or 3, wherein the reduction in the incidence, intensity, and/or duration of neuropathy, or the delaying onset of neuropathy, comprises a reduction in the incidence, intensity, and/or duration of a neuropathic symptom, or delaying onset of a neuropathic symptom.

38. The method of claim 2, wherein the method is effective in at least one of (a)-(d):
(a) a reduction in the intensity and/or duration of an episode of a neuropathic symptom in the subject after administering the nanovesicle formulation, compared to the intensity and/or duration of episodes of the neuropathic symptom in the subject before the formulation was administered;
(b) a reduction in the intensity and/or duration of a neuropathic symptom in the subject, compared to the intensity and/or duration of the neuropathic symptom in a control population;
(c) a reduction in the incidence of neuropathy in the subject, compared to the incidence of neuropathy in a control population; and
(d) a delay in onset of neuropathy in the subject following a triggering event, compared to the period of time prior to onset of neuropathy in a control population following a similar event.

39. The method of claim 3, wherein one or more further doses of the nanovesicle formulation and one or more further doses of the chemotherapeutic agent are administered to the subject.

40. The method of claim 3 or 4, wherein the nanovesicle formulation does not contain the chemotherapeutic agent.

41. The method of claim 5, wherein the neuropathic symptom is associated with
(a) one or more of the following conditions: diabetes, amyotrophic lateral sclerosis (ALS), spinal muscular atrophy (SMA), rheumatoid arthritis, systemic lupus erythematosus (SLE), post-polio syndrome, Guillain-Barre syndrome, chronic inflammatory demyelinating polyneuropathy (CIDP), multifocal motor neuropathy, muscular dystrophy, peripheral nerve injuries, demyelination, acute disseminated leukoencephalitis, progressive multifocal leukoencephalitis (PML), adrenal leukodystrophy, optic neuritis, kidney disease, liver disorder, transverse myelitis, Parkinson's disease, stroke, Alzheimer's disease, Lyme disease, a viral infection, carpal tunnel syndrome, lymphoma, neuroma, multiple myeloma, vitamin B12 deficiency, post-herpetic neuralgia, leprosy, Charcot-Marie-Tooth disease, Fabry disease, critical illness polyneuropathy, Bell's palsy, ulnar nerve palsy, or peroneal nerve palsy; or
(b) treatment with a chemotherapeutic agent selected from the group consisting of oxaliplatin, cisplatin, carboplatin, paclitaxel, docetaxel, cabazitaxel, ixabepilone, vinblastine, vincristine, vindesine, vinorelbine, vincaminol, vineridine, vinburnine, etoposide, thalidomide, lenalidomide, pomalidomide, bortezomib, carfilzomib, ixazomib, eribulin, and suramin.

42. The method of any one of claims 4, 5, 35, and 36, wherein the neuropathic symptom is one or more of the following: pain; hyperalgesia; allodynia; inability to feel pain; inability to feel heat, cold, or physical injury; numbness; hypersensitivity to touch; loss of coordination and proprioception; muscle weakness; muscle wasting; muscle twitching;
cramps; or muscle paralysis.

43. The method of claim 41, wherein the neuropathic symptom is pain.

44. The method of claim 41, wherein the method further includes monitoring the subject's neuropathic symptom.

45. The method of claim 41, wherein the method further includes confirming that the subject's neuropathic symptom is reduced.

46. The method of any of the preceding claims, further comprising administering to the subject one or more additional anti-neuropathy treatments selected from the group consisting of transcutaneous electrical nerve stimulation (TENS), therapeutic plasma exchange (TPE), intravenous immune globulin (IVIG) therapy, physical therapy, a pain reliever, an anti-epileptic agent, a topical treatment, and an anti-depressant agent.

47. The method of any of claims 1-45, further comprising administering to the subject ibuprofen, gabapentin, pregabalin, capsaicin cream, a lidocaine patch, amitriptyline, doxepin, nortriptyline, duloxetine, or venlafaxine.

48. A method of promoting neurogenesis, neuritogenesis, neuroprotection and/or neuroregeneration in a subject, the method comprising:
identifying a subject as being in need of one or more of neurogenesis, neuritogenesis, neuroprotection and neuroregeneration; and
administering to the subject an amount of a nanovesicle formulation sufficient to promote neurogenesis, neuritogenesis, neuroprotection and/or neuroregeneration in the subject,
wherein the nanovesicle formulation comprises (i) a saposin C polypeptide comprising SEQ ID NO: 1 with zero to four amino acid insertions, substitutions, deletions, or combination thereof, and (ii) a phospholipid having a net negative charge at neutral pH.

49. The method of claim 48, wherein the subject has one or more of the following conditions: a neurodegenerative disorder, brain damage, brain injury, spinal cord injury, peripheral nerve damage, multiple sclerosis, or disseminated sclerosis.

50. The method of claim 48, wherein the subject has Parkinson's disease, Alzheimer's disease, or amyotrophic lateral sclerosis.

51. The method of claim 48, wherein the subject is about to undergo anti-cancer chemotherapy.

52. The method of claim 48, further comprising monitoring neurogenesis, neuritogenesis, neuroprotection and/or neuroregeneration in the subject using one or more tests selected from the Weinstein Enhanced Sensory Test (WEST), Semmes-Weinstein Monofilament Test (SWMT), shape-texture identification (STI), magnetic resonance imaging (MRI), computed tomography (CT), intraepidermal nerve fiber density (IENFD), and nerve conduction velocity.

53. The method of claim 48, further comprising confirming that the subject experienced neurogenesis, neuritogenesis, neuroprotection and/or neuroregeneration following administration of the formulation.

54. The method of claim 48, wherein the saposin C polypeptide's amino acid sequence comprises SEQ ID NO: 1 with one or two amino acid insertions, substitutions, deletions, or combination thereof.

55. The method of claim 48, wherein the saposin C polypeptide's amino acid sequence comprises SEQ ID NO: 1.

56. The method of claim 48, wherein the saposin C polypeptide's amino acid sequence consists of SEQ ID NO: 1.

57. The method of any one of claims 48-56, wherein the phospholipid comprises phosphatidylserine.

58. The method of any one of claims 48-56, wherein the phospholipid comprises dioleoyl phosphatidylserine (DOPS) or a salt thereof.

59. The method of claim 58, wherein the phospholipid comprises a sodium salt of DOPS.

60. The method of any one of claims 48-56, wherein the phospholipid comprises phosphoglyceride.

61. The method of claim 57, wherein the phospholipid comprises one or more of dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, or diphytanoyl phosphatidylserine lipid.

62. The method of claim 60, wherein the phosphoglyceride comprises phosphatidate.

63. The method of claim 48, wherein the molar ratio of the phospholipid to the saposin C polypeptide in the nanovesicle formulation is in the range of 8:1 to 20:1.

64. The method of claim 48, wherein the molar ratio of the phospholipid to the saposin C polypeptide in the nanovesicle formulation is in the range of 11:1 to 13:1.

65. The method of claim 48, wherein the molar ratio of the phospholipid to the saposin C polypeptide in the nanovesicle formulation is 12:1.

66. The method of any one of claims 48-65, wherein the nanovesicle formulation is administered intravenously, intra-arterially, intradermally, intramuscularly, intra-cardiacally, intracranially, subcutaneously, intraperitoneally, inhalationally, nasally, orally, or sublingually.

67. The method of any one of claims 48-66, wherein each dose of the nanovesicle formulation administered to the subject contains 0.4 mg/kg to 7 mg/kg of the saposin C polypeptide.

68. The method of claim 67, wherein the nanovesicle formulation is administered intravenously.

69. The method of claim 67 or 68, wherein the nanovesicle formulation is administered at least once a day, once every 2 days, 3 times a week, approximately once every week (every 7 (+/−3) days), or approximately once every 2 weeks (every 14 (+/−3) days).

70. The method of claim 68, wherein multiple doses of the nanovesicle formulation are administered over a period of at least 8 weeks.

71. The method of claim 6, wherein the chemotherapeutic regimen comprises modified FOLFOX7 (mFOLFOX7).

72. The method of claim 6, wherein the antineoplastic agent comprises oxaliplatin.

73. The method of claim 6, wherein the nanovesicle formulation is administered simultaneously with the chemotherapeutic regimen, or within 48 hours before or after administration of the chemotherapeutic regimen begins.

74. The method of claim 6, wherein the nanovesicle formulation is administered intravenously.

75. The method of claim 6, wherein the chemotherapeutic regimen is administered intravenously.

76. The method of claim 6, wherein the gastrointestinal cancer is esophageal cancer, gastric cancer, anal cancer, colorectal cancer, bowel cancer, gallbladder cancer, pancreatic cancer, liver cancer, islet cell cancer, rectal cancer, small intestine cancer, gastrointestinal carcinoid tumors, or gastrointestinal stromal tumors.

77. The method of claim 6, further comprising administering to the subject a biologic treatment for gastrointestinal cancer.

78. The method of claim 6, further comprising administering to the subject an anti-vascular endothelial growth factor (VEGF) monoclonal antibody.

79. The method of claim 6, further comprising administering to the subject an anti-epidermal growth factor receptor (EGFR) antibody.

80. The method of claim 6, further comprising administering to the subject bevacizumab, cetuximab or panitumumab.

81. The method of claim 6, wherein the method comprises
(a) administering 2.4 mg/kg of the nanovesicle formulation over a period of about 45 minutes to about 120 minutes on day 1 of week 1 of treatment, and
(b) after completion of step (a), administering the chemotherapeutic regimen sequentially in the following steps:
(i) 85 mg/m² oxaliplatin infused with 200 mg/m² leucovorin calcium over a period of about 2 hours on day 1 of week 1 of treatment; and
(ii) 2400 mg/m² 5-fluorouracil (5-FU) over a period of about 46 hours on days 1 and 2 of week 1 of treatment.

82. The method of claim 6, wherein the method comprises
(a) administering 2.4 mg/kg of the nanovesicle formulation over a period of about 45 minutes to about 120 minutes on day 1 of week 1 of treatment;
(b) after completion of step (a), administering the chemotherapeutic regimen sequentially in the following steps:
(i) 85 mg/m² oxaliplatin infused with 200 mg/m² leucovorin calcium over a period of about 2 hours on day 1 of week 1 of treatment, and
(ii) 2400 mg/m² 5-FU over a period of about 46 hours on days 1 and 2 of week 1 of treatment; and
(c) administering about 5 mg/kg to about 15 mg/kg of bevacizumab over a period of about 30 to about 90 minutes on day 1 of week 1 of treatment.

83. The method of claim 81 or 82, wherein the method further comprises administering an additional dose of the nanovesicle formulation 3 times a week in week 2 of treatment.

84. The method of claim 83, wherein the method further comprises administering an additional dose of the nanovesicle formulation once every week (every 7 (+/−3) days) in weeks 3 and 4 of treatment.

85. The method of claim 84, wherein the method further comprises administering an additional dose of the nanovesicle formulation every 14 (+/−3) days through weeks 5-23 of treatment.

86. The method of claim 85, wherein multiple doses of the nanovesicle formulation are administered over a period of at least 8 weeks.

87. The method of claim 6, wherein the nanovesicle formulation is administered at least once per week for the first four weeks of treatment.

88. The method of claim 87, wherein the nanovesicle formulation is administered at least three times per week for the first two weeks of treatment.

89. The method of claim 87 or 88, wherein the nanovesicle formulation is administered at least once every 14 (+/−3) days after the first four weeks of treatment.

90. The method of claim 88, wherein the nanovesicle formulation is administered at least once every 14 (+/−3) days in weeks 5-24.

91. The method of claim 89 or 90, wherein the nanovesicle formulation is administered at least once every 28 (+/−3) days after the first 24 weeks of treatment.

92. The method of claim 81 or 82, wherein the nanovesicle formulation is administered repeatedly to the subject, as follows:
week 1: one dose on each of days 1-5;
week 2: one dose every other day for a total of 3 doses;
weeks 3 and 4: one dose each week (every 7 (+/−3) days); and
weeks 5-24: one dose every 14 days (+/−3 days).

93. The method of claim 92, further comprising administering an additional dose of the chemotherapeutic regimen in week 3 and once every 14 days (+/−3 days) in weeks 5-24.

94. The method of claim 82, further comprising administering an additional dose of the bevacizumab in week 3 and once every 14 days (+/−3 days) in weeks 5-24.

95. The method of claim 6, wherein the nanovesicle formulation is not combined with any antineoplastic agent of the chemotherapeutic regimen when the nanovesicle formulation is administered to the subject.

96. The method of any one of claims 71-95, wherein the nanovesicle formulation is administered intravenously, intra-arterially, intradermally, intramuscularly, intra-cardiacally, intracranially, subcutaneously, intraperitoneally, inhalationally, nasally, orally, intrarectally, or sublingually.

97. The method of claim 6, wherein each dose of the nanovesicle formulation administered to the subject contains 0.4 mg/kg to 7 mg/kg of the saposin C polypeptide.

98. The method of claim 97, wherein the treatment increases at least one of the following parameters in the subject relative to a control population treated with the chemotherapeutic regimen and not with the nanovesicle formulation:
(a) objective response rate (ORR);
(b) overall survival (OS);
(c) progression free survival (PFS); and
(d) duration of response (DoR).

99. A kit for the treatment of cancer, the kit comprising, in separate containers,
(a) a first pharmaceutical composition comprising at least one antineoplastic agent; and
(b) a second pharmaceutical composition comprising (i) a saposin C polypeptide comprising SEQ ID NO: 1 with zero to four amino acid insertions, substitutions, deletions, or combination thereof, and (ii) a phospholipid having a net negative charge at neutral pH.

100. The kit of claim 99, wherein the antineoplastic agent is oxaliplatin.

101. A method of treating cancer in a subject in need thereof, the method comprising administering to the subject:
(a) a nanovesicle formulation comprising (i) a saposin C polypeptide comprising SEQ ID NO: 1 with zero to four amino acid insertions, substitutions, deletions, or combination thereof, and (ii) a phospholipid having a net negative charge at neutral pH; and
(b) an immune checkpoint inhibitor.

102. The method of claim 101, wherein the saposin C polypeptide's amino acid sequence comprises SEQ ID NO: 1 with one or two amino acid insertions, substitutions, deletions, or combination thereof.

103. The method of claim 101, wherein the saposin C polypeptide's amino acid sequence comprises SEQ ID NO: 1.

104. The method of claim 101, wherein the saposin C polypeptide's amino acid sequence consists of SEQ ID NO: 1.

105. The method of any one of claim 101-104, wherein the phospholipid comprises phosphatidylserine.

106. The method of any one of claims 101-104, wherein the phospholipid comprises dioleoyl phosphatidylserine (DOPS) or a salt thereof.

107. The method of claim 6, wherein the phospholipid comprises a sodium salt of DOPS.

108. The method of any one of claims 101-104, wherein the phospholipid comprises phosphoglyceride.

109. The method of claim 108, wherein the phosphoglyceride comprises phosphatidate.

110. The method of any of claims 101-107, wherein the phospholipid comprises one or more of dihexanoyl phosphatidylserine lipid, dioctanoyl phosphatidylserine lipid, didecanoyl phosphatidylserine lipid, dilauroyl phosphatidylserine lipid, dimyristoyl phosphatidylserine lipid, dipalmitoyl phosphatidylserine lipid, palmitoyl-oleoyl phosphatidylserine lipid, 1-stearoyl-2-oleoyl phosphatidylserine lipid, or diphytanoyl phosphatidylserine lipid, or a salt of any of the above.

111. The method of any one of claims 101-110, wherein the immune checkpoint inhibitor is an anti-CTLA-4 antibody.

112. The method of claim 111, wherein the anti-CTLA antibody is selected from the group consisting of ipilimumab, tremelimumab, and a combination thereof.

113. The method of any one of claims 101-110, wherein the immune checkpoint inhibitor is an anti-PD-1 antibody.

114. The method of claim 113, wherein the anti-PD-1 antibody is selected from the group consisting of nivolumab, pembrolizumab, pidilizumab, MEDI0680, and combinations thereof.

115. The method of any one of claims 101-110, wherein the immune checkpoint inhibitor is an anti-PD-L1 antibody.

116. The method of claim 115, wherein the anti-PD-L1 antibody is selected from the group consisting of atezolizumab, BMS-936559, MEDI4736, MSB0010718C, and combinations thereof.

117. The method of any one of claims 101-116, wherein the cancer is selected from B cell lymphoma, basal cell carcinoma, bladder cancer, blastoma, brain metastasis, breast cancer, Burkitt's lymphoma, cervical cancer, colon cancer, colorectal cancer, endometrial carcinoma, esophageal cancer, Ewing's sarcoma, fibrosarcoma, follicular lymphoma, gastric cancer, gastroesophageal junction carcinoma, gastrointestinal cancer, glioblastoma, glioma, head and neck cancer, hepatic metastasis, Hodgkin's or non-Hodgkin's lymphoma, kidney cancer, laryngeal cancer, leukemia, liver cancer, lung cancer, lymphoblastic lymphoma, lymphoma, mantle cell lymphoma, metastatic brain tumor, metastatic cancer, myeloma, neuroblastoma, ocular melanoma, oropharyngeal cancer, osteosarcoma, ovarian cancer, pancreatic cancer, prostate cancer, renal cell carcinoma, salivary gland carcinoma, sarcoma, skin cancer, soft tissue sarcoma, solid tumor, squamous cell carcinoma, synovia sarcoma, testicular cancer, thyroid cancer, transitional cell cancer, uveal melanoma, verrucous carcinoma, vulval cancer, and Waldenstrom macroglobulinemia.

118. The method of any one of claims 101-117, wherein the cancer is gastrointestinal cancer.

119. The method of any one of claims 101-118, wherein one or more cells in the cancer express PD-L1 at an elevated level compared to a reference sample.

120. The method of any one of claims 101-118, wherein one or more cells in the cancer express CTLA-4 at an elevated level compared to a reference sample.

121. The method of claim 119 or 120, wherein the reference sample is selected from (a) a noncancerous sample from the subject; or (b) a noncancerous sample from a different subject.

122. The method of claim 121, wherein the one or more cells in the cancer are the same cell type as cells in the reference sample.

123. The method of any one of claims 101-122, further comprising conducting an assay to determine whether the treatment with the nanovesicle formulation and the immune checkpoint inhibitor resulted in an immune response against the subject's tumor that is increased compared to the immune response prior to the treatment.

124. The method of claim 123, wherein the assay measures the level of one or more cytokines in plasma of the subject.

125. The method of claim 123, wherein the assay measures the level of mRNA encoding one or more cytokines in the subject.

126. The method of claim 123, wherein the assay measures the level of tumor-associated M1 or M2 macrophages in the subject.

127. The method of claim 123, wherein the assay detects presentation of PD-L1 on the surface of macrophages in the subject.

128. The method of claim 123, wherein the assay measures the level of activated cytotoxic T cells in the subject.

129. The method of any one of claims 101-128, wherein the nanovesicle formulation does not comprise the immune checkpoint inhibitor.

130. The method of any one of claims 101-129, wherein the nanovesicle formulation is administered intravenously, intra-arterially, intradermally, intramuscularly, intra-cardiacally, intracranially, subcutaneously, intraperitoneally, inhalationally, nasally, orally, intrarectally, or sublingually.

131. A kit for the treatment of cancer, the kit comprising, in separate containers,
(a) a first pharmaceutical composition comprising at least one immune checkpoint inhibitor; and
(b) a second pharmaceutical composition comprising (i) a saposin C polypeptide comprising SEQ ID NO: 1 with zero to four amino acid insertions, substitutions, deletions, or combination thereof, and (ii) a phospholipid having a net negative charge at neutral pH.

132. The kit of claim 131, wherein the at least one immune checkpoint inhibitor is an anti-CTLA-4 antibody, an anti-PD-1 antibody, or an anti-PD-L1 antibody.
